SEQUENCE LISTING (259 sequences)

SEQ ID NO: 1 - DNA, 50 bp, Artificial Sequence
  Description of Artificial Sequence note = synthetic construct
gctgctcgaacttgtttagtaccttcggtccaagaagtcttggcagagga

SEQ ID NO: 2 - DNA, 50 bp, Artificial Sequence
  Description of Artificial Sequence note = synthetic construct
aacttttttaatcgcatctagaattagattatgatttaaaagggaaaact

SEQ ID NO: 3 - DNA, 50 bp, Artificial Sequence
  Description of Artificial Sequence note = synthetic construct
cttgcagattcatatccaaggacaatagaccaatcttttctaaagacaaa

SEQ ID NO: 4 - DNA, 50 bp, Artificial Sequence
  Description of Artificial Sequence note = synthetic construct
aaagatcctcgatatgatctacaagtatgtttgttgagtgatgcggtcca

SEQ ID NO: 5 - DNA, 50 bp, Artificial Sequence
  Description of Artificial Sequence note = synthetic construct
atgcataataacttcgaataaggagaagcttttcatgcgtttccaatagg

SEQ ID NO: 6 - DNA, 50 bp, Artificial Sequence
  Description of Artificial Sequence note = synthetic construct
attcttggcgaatttttaaaacttcctgataagacttttcgctatattct

SEQ ID NO: 7 - DNA, 50 bp, Artificial Sequence
  Description of Artificial Sequence note = synthetic construct
aacgacatttcttgctgcaaagataaaatccctttacccatgaaatccct

SEQ ID NO: 8 - DNA, 50 bp, Artificial Sequence
  Description of Artificial Sequence note = synthetic construct
cgtgatataacctatccgtaaaatgtcctgattagtgaaataatcaggtt

SEQ ID NO: 9 - DNA, 50 bp, Artificial Sequence
  Description of Artificial Sequence note = synthetic construct
gttaacaggatagcacgctcggtatttttttatataaacatgaaaactcg

SEQ ID NO: 10 - DNA, 50 bp, Artificial Sequence
  Description of Artificial Sequence note = synthetic construct
ttccgaaatagaaaatcgcatgcaagatatcgagtatgcgttgttaggta

SEQ ID NO: 11 - DNA, 50 bp, Artificial Sequence
  Description of Artificial Sequence note = synthetic construct
aagctctgatatttgaagactctactgagtatattctgaggcagcttgct

SEQ ID NO: 12 - DNA, 50 bp, Artificial Sequence
  Description of Artificial Sequence note = synthetic construct
aattatgagtttaagtgttctcatcataaaaacatattcatagtatttaa

SEQ ID NO: 13 - DNA, 50 bp, Artificial Sequence
  Description of Artificial Sequence note = synthetic construct
atacttaaaagacaatggattacctataactgtagactcggcttgggaag

SEQ ID NO: 14 - DNA, 50 bp, Artificial Sequence
  Description of Artificial Sequence note = synthetic construct
agcttttgcggcgtcgtatcaaagatatggacaaatcgtatctcgggtta

SEQ ID NO: 15 - DNA, 50 bp, Artificial Sequence
  Description of Artificial Sequence note = synthetic construct
atgttgcatgatgctttatcaaatgacaagcttagatccgtttctcatac

SEQ ID NO: 16 - DNA, 50 bp, Artificial Sequence
  Description of Artificial Sequence note = synthetic construct
ggttttcctcgatgatttgagcgtgtgtagcgctgaagaaaatttgagta

SEQ ID NO: 17 - DNA, 50 bp, Artificial Sequence
  Description of Artificial Sequence note = synthetic construct
atttcattttccgctcgtttaatgagtacaatgaaaatccattgcgtaga

SEQ ID NO: 18 - DNA, 50 bp, Artificial Sequence
  Description of Artificial Sequence note = synthetic construct
tctccgtttctattgcttgagcgtataaagggaaggcttgacagtgctat

SEQ ID NO: 19 - DNA, 50 bp, Artificial Sequence
  Description of Artificial Sequence note = synthetic construct
agcaaagactttttctattcgcagcgctagaggccggtctatttatgata

SEQ ID NO: 20 - DNA, 50 bp, Artificial Sequence
  Description of Artificial Sequence note = synthetic construct
tattctcacagtcagaaattggagtgctggctcgtataaaaaaaagacga

SEQ ID NO: 21 - DNA, 26 bp, Artificial Sequence
  Description of Artificial Sequence note = synthetic construct
tcctccttgcaagctctgcctgtggg

SEQ ID NO: 22 - DNA, 32 bp, Artificial Sequence
  Description of Artificial Sequence note = synthetic construct
ttcctccttgcaagctctgcctgtgggaggaa

SEQ ID NO: 23 - DNA, 34 bp, Artificial Sequence
  Description of Artificial Sequence note = synthetic construct
cttcctccttgcaagctctgcctgtgggaggaag

SEQ ID NO: 24 - DNA, 26 bp, Artificial Sequence
  Description of Artificial Sequence note = synthetic construct
cctccttgcaagctctgcctgtgggg

SEQ ID NO: 25 - DNA, 22 bp, Artificial Sequence
  Description of Artificial Sequence note = synthetic construct
ttcctccttgcaagctctgcct

SEQ ID NO: 26 - DNA, 28 bp, Artificial Sequence
  Description of Artificial Sequence note = synthetic construct
agtatgtggaatgtcgaactcatcggct

SEQ ID NO: 27 - DNA, 27 bp, Artificial Sequence
  Description of Artificial Sequence note = synthetic construct
ccgtatgtggaatgtcgaactcatcgg

SEQ ID NO: 28 - DNA, 26 bp, Artificial Sequence
  Description of Artificial Sequence note = synthetic construct
gtgatagggaaagtatgtggaatgtc

SEQ ID NO: 29 - DNA, 23 bp, Artificial Sequence
  Description of Artificial Sequence note = synthetic construct
agggaaagtatgtggaatgtcct

SEQ ID NO: 30 - DNA, 26 bp, Artificial Sequence
  Description of Artificial Sequence note = synthetic construct
aaagtatgtggaatgtcgaactcttt

SEQ ID NO: 31 - DNA, 29 bp, Artificial Sequence
  Description of Artificial Sequence note = synthetic construct
acgtgcgggcgatttgccttaaccccacc

SEQ ID NO: 32 - DNA, 33 bp, Artificial Sequence
  Description of Artificial Sequence note = synthetic construct
cgtgcgggcgatttgccttaaccccaccgcacg

SEQ ID NO: 33 - DNA, 35 bp, Artificial Sequence
  Description of Artificial Sequence note = synthetic construct
aacgtgcgggcgatttgccttaaccccaccgcacg

SEQ ID NO: 34 - DNA, 21 bp, Artificial Sequence
  Description of Artificial Sequence note = synthetic construct
aacgtgcgggcgatttgcctt

SEQ ID NO: 35 - DNA, 37 bp, Artificial Sequence
  Description of Artificial Sequence note = synthetic construct
tggcgaatttttaaaacttcctgataagacttttcgc

SEQ ID NO: 36 - DNA, 35 bp, Artificial Sequence
  Description of Artificial Sequence note = synthetic construct
gcgaatttttaaaacttcctgataagacttttcgc

SEQ ID NO: 37 - DNA, 27 bp, Artificial Sequence
  Description of Artificial Sequence note = synthetic construct
ccgtatgtggaatgtcgaactcatcgg

SEQ ID NO: 38 - DNA, 25 bp, Artificial Sequence
  Description of Artificial Sequence note = synthetic construct
cuagcgguaaaacugcuuacugguc

SEQ ID NO: 39 - DNA, 25 bp, Artificial Sequence
  Description of Artificial Sequence note = synthetic construct
agauaaaauccauacagaagcaaca

SEQ ID NO: 40 - DNA, 25 bp, Artificial Sequence
  Description of Artificial Sequence note = synthetic construct
cguacuucuuuuaggagaaaaaauc

SEQ ID NO: 41 - DNA, 25 bp, Artificial Sequence
  Description of Artificial Sequence note = synthetic construct
uauaaugcuagaaaaauccugagua

SEQ ID NO: 42 - DNA, 25 bp, Artificial Sequence
  Description of Artificial Sequence note = synthetic construct
aggaucacuucuccucaacaacuuu

SEQ ID NO: 43 - DNA, 25 bp, Artificial Sequence
  Description of Artificial Sequence note = synthetic construct
uucaucuuggauagaguuaguuuuu

SEQ ID NO: 44 - DNA, 25 bp, Artificial Sequence
  Description of Artificial Sequence note = synthetic construct
agaacuaagucuucugcuuacaaug

SEQ ID NO: 45 - DNA, 25 bp, Artificial Sequence
  Description of Artificial Sequence note = synthetic construct
cucuugcauauuacgagcuuuuuau

SEQ ID NO: 46 - DNA, 25 bp, Artificial Sequence
  Description of Artificial Sequence note = synthetic construct
aaaccuccccaaccaaacucuacaa

SEQ ID NO: 47 - DNA, 25 bp, Artificial Sequence
  Description of Artificial Sequence note = synthetic construct
aaagaguuucaaucgauccccuaua

SEQ ID NO: 48 - DNA, 25 bp, Artificial Sequence
  Description of Artificial Sequence note = synthetic construct
aauccgcauauauuuuggccgcuag

SEQ ID NO: 49 - DNA, 25 bp, Artificial Sequence
  Description of Artificial Sequence note = synthetic construct
gacguuagagaaacgauagauaagu

SEQ ID NO: 50 - DNA, 25 bp, Artificial Sequence
  Description of Artificial Sequence note = synthetic construct
cugauucagagaagaaucgccaauu

SEQ ID NO: 51 - DNA, 25 bp, Artificial Sequence
  Description of Artificial Sequence note = synthetic construct
aucugauuucuuaauagagauacuu

SEQ ID NO: 52 - DNA, 25 bp, Artificial Sequence
  Description of Artificial Sequence note = synthetic construct
cgcaucauguguuccggaguuucuu

SEQ ID NO: 53 - DNA, 25 bp, Artificial Sequence
  Description of Artificial Sequence note = synthetic construct
uguccuccuauaacgaaaaucuucu

SEQ ID NO: 54 - DNA, 25 bp, Artificial Sequence
  Description of Artificial Sequence note = synthetic construct
acaacagcuuuuugaacuuuuuaag

SEQ ID NO: 55 - DNA, 25 bp, Artificial Sequence
  Description of Artificial Sequence note = synthetic construct
caaaagagcugauccuccgucagcu

SEQ ID NO: 56 - DNA, 25 bp, Artificial Sequence
  Description of Artificial Sequence note = synthetic construct
cauauauauaucuauuauauauaua

SEQ ID NO: 57 - DNA, 25 bp, Artificial Sequence
  Description of Artificial Sequence note = synthetic construct
uauuuagggauuugauuuuacgaga

SEQ ID NO: 58 - DNA, 25 bp, Artificial Sequence
  Description of Artificial Sequence note = synthetic construct
aagggcuucuuccugggacgaacgu

SEQ ID NO: 59 - DNA, 25 bp, Artificial Sequence
  Description of Artificial Sequence note = synthetic construct
uuuucuuaucuucuuuacgagaaua

SEQ ID NO: 60 - DNA, 25 bp, Artificial Sequence
  Description of Artificial Sequence note = synthetic construct
agaaaauuuuguuauggcucgagca

SEQ ID NO: 61 - DNA, 25 bp, Artificial Sequence
  Description of Artificial Sequence note = synthetic construct
uugaacgacauguucucgauuaagg

SEQ ID NO: 62 - DNA, 25 bp, Artificial Sequence
  Description of Artificial Sequence note = synthetic construct
cugcuuuuacuugcaagacauuccu

SEQ ID NO: 63 - DNA, 25 bp, Artificial Sequence
  Description of Artificial Sequence note = synthetic construct
caggccauuaauugcuacaggacau

SEQ ID NO: 64 - DNA, 25 bp, Artificial Sequence
  Description of Artificial Sequence note = synthetic construct
cuugucuggcuuuaacuaggacgca

SEQ ID NO: 65 - DNA, 25 bp, Artificial Sequence
  Description of Artificial Sequence note = synthetic construct
gugccgccagaaaaagauagcgagc

SEQ ID NO: 66 - DNA, 25 bp, Artificial Sequence
  Description of Artificial Sequence note = synthetic construct
acaaagagagcuaauuauacaauuu

SEQ ID NO: 67 - DNA, 25 bp, Artificial Sequence
  Description of Artificial Sequence note = synthetic construct
agagguaagaaugaaaaaacucuug

SEQ ID NO: 68 - DNA, 25 bp, Artificial Sequence
  Description of Artificial Sequence note = synthetic construct
cggaauucuaugggaagguuucggc

SEQ ID NO: 69 - DNA, 25 bp, Artificial Sequence
  Description of Artificial Sequence note = synthetic construct
ggagauccuugcgauccuugcacca

SEQ ID NO: 70 - DNA, 25 bp, Artificial Sequence
  Description of Artificial Sequence note = synthetic construct
cuuggugugacgcuaucagcaugcg

SEQ ID NO: 71 - DNA, 25 bp, Artificial Sequence
  Description of Artificial Sequence note = synthetic construct
uauggguuacuauggugacuuuguu

SEQ ID NO: 72 - DNA, 25 bp, Artificial Sequence
  Description of Artificial Sequence note = synthetic construct
uucgaccguguuuugcaaacagaug

SEQ ID NO: 73 - DNA, 25 bp, Artificial Sequence
  Description of Artificial Sequence note = synthetic construct
ugaauaaagaauuccaaaugggugc

SEQ ID NO: 74 - DNA, 25 bp, Artificial Sequence
  Description of Artificial Sequence note = synthetic construct
caagccuacaacugcuacaggcaau

SEQ ID NO: 75 - DNA, 25 bp, Artificial Sequence
  Description of Artificial Sequence note = synthetic construct
gcugcagcuccauccacuuguacag

SEQ ID NO: 76 - DNA, 25 bp, Artificial Sequence
  Description of Artificial Sequence note = synthetic construct
caagagagaauccugcuuacggccg

SEQ ID NO: 77 - DNA, 25 bp, Artificial Sequence
  Description of Artificial Sequence note = synthetic construct
acauaugcaggaugcugagauguuu

SEQ ID NO: 78 - DNA, 30 bp, Artificial Sequence
  Description of Artificial Sequence note = synthetic construct
accgatatagggtttgaatttgtcgttgag

SEQ ID NO: 79 - DNA, 30 bp, Artificial Sequence
  Description of Artificial Sequence note = synthetic construct
tttgaaatcgtaaacggcggacaagccgag

SEQ ID NO: 80 - DNA, 30 bp, Artificial Sequence
  Description of Artificial Sequence note = synthetic construct
agaagaaacggcgtggaacgtaccgttttc

SEQ ID NO: 81 - DNA, 30 bp, Artificial Sequence
  Description of Artificial Sequence note = synthetic construct
ctgattttccgccttcagatattgcgtcac

SEQ ID NO: 82 - DNA, 30 bp, Artificial Sequence
  Description of Artificial Sequence note = synthetic construct
gtttatcttttcgcccttgttttcgttcac

SEQ ID NO: 83 - DNA, 30 bp, Artificial Sequence
  Description of Artificial Sequence note = synthetic construct
cttttttgtgttgacggaatatttactgtt

SEQ ID NO: 84 - DNA, 30 bp, Artificial Sequence
  Description of Artificial Sequence note = synthetic construct
gttccactttctgtaacgggcataatctgc

SEQ ID NO: 85 - DNA, 30 bp, Artificial Sequence
  Description of Artificial Sequence note = synthetic construct
cgctatcctccagccgccgaagtcgtagcc

SEQ ID NO: 86 - DNA, 30 bp, Artificial Sequence
  Description of Artificial Sequence note = synthetic construct
gaccgacaccctggggtggatggaatgcgt

SEQ ID NO: 87 - DNA, 30 bp, Artificial Sequence
  Description of Artificial Sequence note = synthetic construct
acggatgtttctgaaataatcgcttaccgt

SEQ ID NO: 88 - DNA, 30 bp, Artificial Sequence
  Description of Artificial Sequence note = synthetic construct
gcttattttgtctttttttgtaccggttgg

SEQ ID NO: 89 - DNA, 30 bp, Artificial Sequence
  Description of Artificial Sequence note = synthetic construct
ttccggataatcgtgggtaatgcgttcggc

SEQ ID NO: 90 - DNA, 30 bp, Artificial Sequence
  Description of Artificial Sequence note = synthetic construct
ggcgtaggctaaatccgcctgcacatacgg

SEQ ID NO: 91 - DNA, 30 bp, Artificial Sequence
  Description of Artificial Sequence note = synthetic construct
gccgcggccattgccttcacttgccgcctg

SEQ ID NO: 92 - DNA, 30 bp, Artificial Sequence
  Description of Artificial Sequence note = synthetic construct
cgctgcggaagagaagagaaggttttttgc

SEQ ID NO: 93 - DNA, 30 bp, Artificial Sequence
  Description of Artificial Sequence note = synthetic construct
gggctggattcattttcggctccttattcg

SEQ ID NO: 94 - DNA, 30 bp, Artificial Sequence
  Description of Artificial Sequence note = synthetic construct
gtttaaccggttaaaaaaaagattttcact

SEQ ID NO: 95 - DNA, 30 bp, Artificial Sequence
  Description of Artificial Sequence note = synthetic construct
gatgttgaagggcggattatatcgggttcc

SEQ ID NO: 96 - DNA, 30 bp, Artificial Sequence
  Description of Artificial Sequence note = synthetic construct
gggcggtgtttcaacacaatatggcggatg

SEQ ID NO: 97 - DNA, 30 bp, Artificial Sequence
  Description of Artificial Sequence note = synthetic construct
aacaaaaaccggtacgggttgccccgcccc

SEQ ID NO: 98 - DNA, 30 bp, Artificial Sequence
  Description of Artificial Sequence note = synthetic construct
ggctcaaagggaacggttccctaagacgcc

SEQ ID NO: 99 - DNA, 30 bp, Artificial Sequence
  Description of Artificial Sequence note = synthetic construct
caagcaccgggcggatcggttccgtaccat

SEQ ID NO: 100 - DNA, 30 bp, Artificial Sequence
  Description of Artificial Sequence note = synthetic construct
ttgtaccgtctgcggcccgccgccttgtcc

SEQ ID NO: 101 - DNA, 30 bp, Artificial Sequence
  Description of Artificial Sequence note = synthetic construct
tgatttttgttaatccgctatacgtctgat

SEQ ID NO: 102 - DNA, 30 bp, Artificial Sequence
  Description of Artificial Sequence note = synthetic construct
tgatgccgaatctttggaagaagtcttgaa

SEQ ID NO: 103 - DNA, 30 bp, Artificial Sequence
  Description of Artificial Sequence note = synthetic construct
acaatagaagcaggcaattggaatagggtt

SEQ ID NO: 104 - DNA, 30 bp, Artificial Sequence
  Description of Artificial Sequence note = synthetic construct
ttcttttcataagaaacagccgcaaagacc

SEQ ID NO: 105 - DNA, 30 bp, Artificial Sequence
  Description of Artificial Sequence note = synthetic construct
gtgatctttgcggctgtctgttttctgtcc

SEQ ID NO: 106 - DNA, 30 bp, Artificial Sequence
  Description of Artificial Sequence note = synthetic construct
gtcagaaccggtagcctacgccgatttgtc

SEQ ID NO: 107 - DNA, 30 bp, Artificial Sequence
  Description of Artificial Sequence note = synthetic construct
cgctgtggttgccgtactgtttggaaccgg

SEQ ID NO: 108 - DNA, 30 bp, Artificial Sequence
  Description of Artificial Sequence note = synthetic construct
tgtagctgtaacgtgccaagccgttccagc

SEQ ID NO: 109 - DNA, 30 bp, Artificial Sequence
  Description of Artificial Sequence note = synthetic construct
cggcaacccggcgggtgtgcggcatattgc

SEQ ID NO: 110 - DNA, 30 bp, Artificial Sequence
  Description of Artificial Sequence note = synthetic construct
gtgcacccgtcttgccggttgctgcagccg

SEQ ID NO: 111 - DNA, 30 bp, Artificial Sequence
  Description of Artificial Sequence note = synthetic construct
cgttgccgaattcgacatccacccccagac

SEQ ID NO: 112 - DNA, 30 bp, Artificial Sequence
  Description of Artificial Sequence note = synthetic construct
tccgcctatacgcctgctactttcacgctg

SEQ ID NO: 113 - DNA, 31 bp, Artificial Sequence
  Description of Artificial Sequence note = synthetic construct
tccgcctatacgcctgctactttcacgctgg

SEQ ID NO: 114 - DNA, 32 bp, Artificial Sequence
  Description of Artificial Sequence note = synthetic construct
tccgcctatacgcctgctactttcacgctgga

SEQ ID NO: 115 - DNA, 25 bp, Artificial Sequence
  Description of Artificial Sequence note = synthetic construct
cctatacgcctgctactttcacggg

SEQ ID NO: 116 - DNA, 26 bp, Artificial Sequence
  Description of Artificial Sequence note = synthetic construct
cctatacgcctgctactttcacgagg

SEQ ID NO: 117 - DNA, 26 bp, Artificial Sequence
  Description of Artificial Sequence note = synthetic construct
cctatacgcctgctactttcacgctg

SEQ ID NO: 118 - DNA, 27 bp, Artificial Sequence
  Description of Artificial Sequence note = synthetic construct
ccatatacgcctgctactttcacgtgg

SEQ ID NO: 119 - DNA, 30 bp, Artificial Sequence
  Description of Artificial Sequence note = synthetic construct
cgtgaaagtagcaggcgtataggcggactt

SEQ ID NO: 120 - DNA, 24 bp, Artificial Sequence
  Description of Artificial Sequence note = synthetic construct
cgcagtcagaaacgcgaacatacc

SEQ ID NO: 121 - DNA, 27 bp, Artificial Sequence
  Description of Artificial Sequence note = synthetic construct
cagtcagaaacgcgaacataccagctg

SEQ ID NO: 122 - DNA, 26 bp, Artificial Sequence
  Description of Artificial Sequence note = synthetic construct
aacgcagtcagaaacgcgaacatacc

SEQ ID NO: 123 - DNA, 19 bp, Artificial Sequence
  Description of Artificial Sequence note = synthetic construct
gcgagtgataccgatccat

SEQ ID NO: 124 - DNA, 23 bp, Artificial Sequence
  Description of Artificial Sequence note = synthetic construct
cgaggaagccgatatgcgactcg

SEQ ID NO: 125 - DNA, 20 bp, Artificial Sequence
  Description of Artificial Sequence note = synthetic construct
cgcctatacgcctgctactt

SEQ ID NO: 126 - DNA, 19 bp, Artificial Sequence
  Description of Artificial Sequence note = synthetic construct
gcctgctactttcacgctg

SEQ ID NO: 127 - DNA, 27 bp, Artificial Sequence
  Description of Artificial Sequence note = synthetic construct
ccgcccttcaacatcagtgaaaatctt

SEQ ID NO: 128 - DNA, 18 bp, Artificial Sequence
  Description of Artificial Sequence note = synthetic construct
ccgcccttcaacatcagt

SEQ ID NO: 129 - DNA, 29 bp, Artificial Sequence
  Description of Artificial Sequence note = synthetic construct
tccgtccttcaacatcagtgaaaatcgga

SEQ ID NO: 130 - DNA, 23 bp, Artificial Sequence
  Description of Artificial Sequence note = synthetic construct
cgtccttcaacatcagtgaaaat

SEQ ID NO: 131 - DNA, 27 bp, Artificial Sequence
  Description of Artificial Sequence note = synthetic construct
ctgatataatccgtccttcaacatcag

SEQ ID NO: 132 - DNA, 25 bp, Artificial Sequence
  Description of Artificial Sequence note = synthetic construct
cgtccttcaacatcagtgaaaatcg

SEQ ID NO: 133 - DNA, 25 bp, Artificial Sequence
  Description of Artificial Sequence note = synthetic construct
cgcctatacgcctgctactttcacg

SEQ ID NO: 134 - DNA, 25 bp, Artificial Sequence
  Description of Artificial Sequence note = synthetic construct
cugcagaugcccgacggucuuuaua

SEQ ID NO: 135 - DNA, 25 bp, Artificial Sequence
  Description of Artificial Sequence note = synthetic construct
gcggauuaacaaaaaucaggacaag

SEQ ID NO: 136 - DNA, 25 bp, Artificial Sequence
  Description of Artificial Sequence note = synthetic construct
gggcgggccgcaggcaguacaaaug

SEQ ID NO: 137 - DNA, 25 bp, Artificial Sequence
  Description of Artificial Sequence note = synthetic construct
guacggaaccgauccgcccggugcu

SEQ ID NO: 138 - DNA, 25 bp, Artificial Sequence
  Description of Artificial Sequence note = synthetic construct
ugggcgccuuagggaaccguucccu

SEQ ID NO: 139 - DNA, 25 bp, Artificial Sequence
  Description of Artificial Sequence note = synthetic construct
uugagccggggcggggcaacgacgu

SEQ ID NO: 140 - DNA, 25 bp, Artificial Sequence
  Description of Artificial Sequence note = synthetic construct
accgguuuuuguucauccgccauau

SEQ ID NO: 141 - DNA, 25 bp, Artificial Sequence
  Description of Artificial Sequence note = synthetic construct
ccagcccccaaaaaaccuucucuuc

SEQ ID NO: 142 - DNA, 25 bp, Artificial Sequence
  Description of Artificial Sequence note = synthetic construct
ucuucucuucucuucucuucucuuc

SEQ ID NO: 143 - DNA, 25 bp, Artificial Sequence
  Description of Artificial Sequence note = synthetic construct
ucuuccgcagcgcaggcggcgggug

SEQ ID NO: 144 - DNA, 25 bp, Artificial Sequence
  Description of Artificial Sequence note = synthetic construct
aagaccauggccgcggcccguaugu

SEQ ID NO: 145 - DNA, 25 bp, Artificial Sequence
  Description of Artificial Sequence note = synthetic construct
gcaggcggauuuagccuacgccuac

SEQ ID NO: 146 - DNA, 25 bp, Artificial Sequence
  Description of Artificial Sequence note = synthetic construct
gaacacauuacccacgauuauccgg

SEQ ID NO: 147 - DNA, 25 bp, Artificial Sequence
  Description of Artificial Sequence note = synthetic construct
aacaaaccgcuccaaaaaaagcaca

SEQ ID NO: 148 - DNA, 25 bp, Artificial Sequence
  Description of Artificial Sequence note = synthetic construct
auuaagcacgguaagcgauuauuuc

SEQ ID NO: 149 - DNA, 25 bp, Artificial Sequence
  Description of Artificial Sequence note = synthetic construct
agaaacauccguacgcauuccaucc

SEQ ID NO: 150 - DNA, 25 bp, Artificial Sequence
  Description of Artificial Sequence note = synthetic construct
accccagggugucggucggcuacga

SEQ ID NO: 151 - DNA, 25 bp, Artificial Sequence
  Description of Artificial Sequence note = synthetic construct
cuucggcggcuggaggauagcggca

SEQ ID NO: 152 - DNA, 25 bp, Artificial Sequence
  Description of Artificial Sequence note = synthetic construct
gauuaugcccguuacagaaagugga

SEQ ID NO: 153 - DNA, 25 bp, Artificial Sequence
  Description of Artificial Sequence note = synthetic construct
acaacaauaaauauuccguuaacau

SEQ ID NO: 154 - DNA, 80 bp, Artificial Sequence
  Description of Artificial Sequence note = synthetic construct
gtatttgccgctttgagttcataacgtccggcgagttgtctcatccaccaccggaaaaaagaatcctgctgaaccaagcc

SEQ ID NO: 155 - DNA, 22 bp, Artificial Sequence
  Description of Artificial Sequence note = synthetic construct
aacgtccggcgagttgtctcat

SEQ ID NO: 156 - DNA, 33 bp, Artificial Sequence
  Description of Artificial Sequence note = synthetic construct
cgtccggcgagttgtctcatccaccaccggacg

SEQ ID NO: 157 - DNA, 86 bp, Artificial Sequence
  Description of Artificial Sequence note = synthetic construct
cggtattagtatttgccgctttgagttctgatcgagagctcatatgaccacggccggctgaatcctgctgaaccaagccttatgat

SEQ ID NO: 158 - DNA, 86 bp, Artificial Sequence
  Description of Artificial Sequence note = synthetic construct
cggtattagtatttgccgctttgagtactgatcgagagctcatatgaccacggccggctgtatcctgctgaaccaagccttatgat

SEQ ID NO: 159 - DNA, 20 bp, Artificial Sequence
  Description of Artificial Sequence note = synthetic construct
cgagagctcatatgaccacg

SEQ ID NO: 160 - DNA, 28 bp, Artificial Sequence
  Description of Artificial Sequence note = synthetic construct
atcgagagctcatatgaccacggccgat

SEQ ID NO: 161 - DNA, 24 bp, Artificial Sequence
  Description of Artificial Sequence note = synthetic construct
atcgagagctcatatgaccacgat

SEQ ID NO: 162 - DNA, 26 bp, Artificial Sequence
  Description of Artificial Sequence note = synthetic construct
gatcgagagctcatatgaccacgatc

SEQ ID NO: 163 - DNA, 85 bp, Artificial Sequence
  Description of Artificial Sequence note = synthetic construct
aggcgatttaaaaaccaaggtcgttcttgatcgagagctcatatgaccacggccggctccattagggtgttggatcaatttcttc

SEQ ID NO: 164 - DNA, 85 bp, Artificial Sequence
  Description of Artificial Sequence note = synthetic construct
aggcgatttaaaaaccaaggtcgatcttgatcgagagctcatatgaccacggccggctccataagggtgttggatcaatttcttc

SEQ ID NO: 165 - DNA, 25 bp, Artificial Sequence
  Description of Artificial Sequence note = synthetic construct
gcccgguacccagcuuuuguucccu

SEQ ID NO: 166 - DNA, 25 bp, Artificial Sequence
  Description of Artificial Sequence note = synthetic construct
uuagugaggguuaauugcgcgcuug

SEQ ID NO: 167 - DNA, 25 bp, Artificial Sequence
  Description of Artificial Sequence note = synthetic construct
gcguaaucauggucauagcuguuuc

SEQ ID NO: 168 - DNA, 25 bp, Artificial Sequence
  Description of Artificial Sequence note = synthetic construct
cugugugaaauuguuauccgcucac

SEQ ID NO: 169 - DNA, 25 bp, Artificial Sequence
  Description of Artificial Sequence note = synthetic construct
aauuccacacaacauacgagccggg

SEQ ID NO: 170 - DNA, 25 bp, Artificial Sequence
  Description of Artificial Sequence note = synthetic construct
agcauaaaguguaaagccuggggug

SEQ ID NO: 171 - DNA, 25 bp, Artificial Sequence
  Description of Artificial Sequence note = synthetic construct
ccuaaugagugagcuaacucacauu

SEQ ID NO: 172 - DNA, 25 bp, Artificial Sequence
  Description of Artificial Sequence note = synthetic construct
aauugcguugcgcucacugcccgcu

SEQ ID NO: 173 - DNA, 25 bp, Artificial Sequence
  Description of Artificial Sequence note = synthetic construct
uuccagucgggaaaccugucgugcc

SEQ ID NO: 174 - DNA, 25 bp, Artificial Sequence
  Description of Artificial Sequence note = synthetic construct
agcugcauuaaugaaucggccaacg

SEQ ID NO: 175 - DNA, 25 bp, Artificial Sequence
  Description of Artificial Sequence note = synthetic construct
acgcugcgcguaaccaccacacccg

SEQ ID NO: 176 - DNA, 25 bp, Artificial Sequence
  Description of Artificial Sequence note = synthetic construct
ccgcgcuuaaugcgccgcuacaggg

SEQ ID NO: 177 - DNA, 25 bp, Artificial Sequence
  Description of Artificial Sequence note = synthetic construct
cgcgucccauucgccauucaggcug

SEQ ID NO: 178 - DNA, 25 bp, Artificial Sequence
  Description of Artificial Sequence note = synthetic construct
cgcaacuguugggaagggcgaucgg

SEQ ID NO: 179 - DNA, 25 bp, Artificial Sequence
  Description of Artificial Sequence note = synthetic construct
ugcgggccucuucgcuauuacgcca

SEQ ID NO: 180 - DNA, 25 bp, Artificial Sequence
  Description of Artificial Sequence note = synthetic construct
gcuggcgaaagggggaugugcugca

SEQ ID NO: 181 - DNA, 25 bp, Artificial Sequence
  Description of Artificial Sequence note = synthetic construct
aggcgauuaaguuggguaacgccag

SEQ ID NO: 182 - DNA, 25 bp, Artificial Sequence
  Description of Artificial Sequence note = synthetic construct
gguuuucccagucacgacguuguaa

SEQ ID NO: 183 - DNA, 25 bp, Artificial Sequence
  Description of Artificial Sequence note = synthetic construct
aacgacggccagugagcgcgcguaa

SEQ ID NO: 184 - DNA, 25 bp, Artificial Sequence
  Description of Artificial Sequence note = synthetic construct
uacgacucacuauagggcgaauugg

SEQ ID NO: 185 - DNA, 27 bp, Artificial Sequence
  Description of Artificial Sequence note = synthetic construct
atcgcatgcaagatatcgagtatgcgt

SEQ ID NO: 186 - DNA, 27 bp, Artificial Sequence
  Description of Artificial Sequence note = synthetic construct
ctcataattagcaagctgcctcagaat

SEQ ID NO: 187 - DNA, 27 bp, Artificial Sequence
  Description of Artificial Sequence note = synthetic construct
agtatttgccgctttgagttctgcttc

SEQ ID NO: 188 - DNA, 27 bp, Artificial Sequence
  Description of Artificial Sequence note = synthetic construct
gatcataaggcttggttcagcaggatt

SEQ ID NO: 189 - DNA, 27 bp, Artificial Sequence
  Description of Artificial Sequence note = synthetic construct
atcgcatgcaagatatcgagtatgcgt

SEQ ID NO: 190 - DNA, 27 bp, Artificial Sequence
  Description of Artificial Sequence note = synthetic construct
ctcataattagcaagctgcctcagaat

SEQ ID NO: 191 - DNA, 27 bp, Artificial Sequence
  Description of Artificial Sequence note = synthetic construct
aaccaaggtcgatgtgatagggaaagt

SEQ ID NO: 192 - DNA, 30 bp, Artificial Sequence
  Description of Artificial Sequence note = synthetic construct
tcgtttctctaacgtctttgtttctagatg

SEQ ID NO: 193 - DNA, 27 bp, Artificial Sequence
  Description of Artificial Sequence note = synthetic construct
aaaaccaaggtcgatgtgatagggaaa

SEQ ID NO: 194 - DNA, 29 bp, Artificial Sequence
  Description of Artificial Sequence note = synthetic construct
tctctaacgtctttgtttctagatgaagg

SEQ ID NO: 195 - DNA, 30 bp, Artificial Sequence
  Description of Artificial Sequence note = synthetic construct
cggggttatcttaaaagggattgcagcttg

SEQ ID NO: 196 - DNA, 27 bp, Artificial Sequence
  Description of Artificial Sequence note = synthetic construct
tcaacgaagaggttttgtcttcgtaac

SEQ ID NO: 197 - DNA, 23 bp, Artificial Sequence
  Description of Artificial Sequence note = synthetic construct
gcttttcatgcgtttccaatagg

SEQ ID NO: 198 - DNA, 24 bp, Artificial Sequence
  Description of Artificial Sequence note = synthetic construct
ctttgcagcaagaaatgtcgttag

SEQ ID NO: 199 - DNA, 28 bp, Artificial Sequence
  Description of Artificial Sequence note = synthetic construct
cggtattagtatttgccgctttgagttc

SEQ ID NO: 200 - DNA, 27 bp, Artificial Sequence
  Description of Artificial Sequence note = synthetic construct
atcataaggcttggttcagcaggattc

SEQ ID NO: 201 - DNA, 26 bp, Artificial Sequence
  Description of Artificial Sequence note = synthetic construct
atttgccgctttgagttctgcttcct

SEQ ID NO: 202 - DNA, 27 bp, Artificial Sequence
  Description of Artificial Sequence note = synthetic construct
atcataaggcttggttcagcaggattc

SEQ ID NO: 203 - DNA, 27 bp, Artificial Sequence
  Description of Artificial Sequence note = synthetic construct
aggcgatttaaaaaccaaggtcgatgt

SEQ ID NO: 204 - DNA, 27 bp, Artificial Sequence
  Description of Artificial Sequence note = synthetic construct
gaagaaattgatccaacacccttatcg

SEQ ID NO: 205 - DNA, 24 bp, Artificial Sequence
  Description of Artificial Sequence note = synthetic construct
tgttccgagtcaaaacagcaagtc

SEQ ID NO: 206 - DNA, 24 bp, Artificial Sequence
  Description of Artificial Sequence note = synthetic construct
gccggaactggtttcatctgatta

SEQ ID NO: 207 - DNA, 27 bp, Artificial Sequence
  Description of Artificial Sequence note = synthetic construct
aatttgttccgagtcaaaacagcaagt

SEQ ID NO: 208 - DNA, 27 bp, Artificial Sequence
  Description of Artificial Sequence note = synthetic construct
ggaactggtttcatctgattactttcc

SEQ ID NO: 209 - DNA, 22 bp, Artificial Sequence
  Description of Artificial Sequence note = synthetic construct
agccaccctcagaaggtcaaac

SEQ ID NO: 210 - DNA, 23 bp, Artificial Sequence
  Description of Artificial Sequence note = synthetic construct
aacgagccgaaatcactgacttt

SEQ ID NO: 211 - DNA, 24 bp, Artificial Sequence
  Description of Artificial Sequence note = synthetic construct
ctatgcccatggtttcgactttgt

SEQ ID NO: 212 - DNA, 20 bp, Artificial Sequence
  Description of Artificial Sequence note = synthetic construct
gtaatcgacaccggcgatga

SEQ ID NO: 213 - DNA, 20 bp, Artificial Sequence
  Description of Artificial Sequence note = synthetic construct
tgcccatggtttcgactttg

SEQ ID NO: 214 - DNA, 21 bp, Artificial Sequence
  Description of Artificial Sequence note = synthetic construct
gtaatcgacaccggcgatgat

SEQ ID NO: 215 - DNA, 24 bp, Artificial Sequence
  Description of Artificial Sequence note = synthetic construct
aattggagactgattgggtgtttg

SEQ ID NO: 216 - DNA, 24 bp, Artificial Sequence
  Description of Artificial Sequence note = synthetic construct
aatacgagggcggtaagttttttt

SEQ ID NO: 217 - DNA, 22 bp, Artificial Sequence
  Description of Artificial Sequence note = synthetic construct
cggctcagttggatttgtctga

SEQ ID NO: 218 - DNA, 21 bp, Artificial Sequence
  Description of Artificial Sequence note = synthetic construct
gatgcgcgggactgtattacc

SEQ ID NO: 219 - DNA, 24 bp, Artificial Sequence
  Description of Artificial Sequence note = synthetic construct
ttctttttgttcttgctcggcaga

SEQ ID NO: 220 - DNA, 21 bp, Artificial Sequence
  Description of Artificial Sequence note = synthetic construct
gcggtgtacctgatggttttt

SEQ ID NO: 221 - DNA, 18 bp, Artificial Sequence
  Description of Artificial Sequence note = synthetic construct
ttgaaacaccgcccggaa

SEQ ID NO: 222 - DNA, 23 bp, Artificial Sequence
  Description of Artificial Sequence note = synthetic construct
tttcggctccttattcggtttaa

SEQ ID NO: 223 - DNA, 22 bp, Artificial Sequence
  Description of Artificial Sequence note = synthetic construct
gttcatccgccatattgtgttg

SEQ ID NO: 224 - DNA, 24 bp, Artificial Sequence
  Description of Artificial Sequence note = synthetic construct
cactgatgttgaaggacggattat

SEQ ID NO: 225 - DNA, 23 bp, Artificial Sequence
  Description of Artificial Sequence note = synthetic construct
ttcggctccttattcggtttaac

SEQ ID NO: 226 - DNA, 19 bp, Artificial Sequence
  Description of Artificial Sequence note = synthetic construct
ccgatataatccgcccttc

SEQ ID NO: 227 - DNA, 20 bp, Artificial Sequence
  Description of Artificial Sequence note = synthetic construct
ttcggctccttattcggttt

SEQ ID NO: 228 - DNA, 22 bp, Artificial Sequence
  Description of Artificial Sequence note = synthetic construct
acccgatataatccgtccttca

SEQ ID NO: 229 - DNA, 22 bp, Artificial Sequence
  Description of Artificial Sequence note = synthetic construct
cggctccttattcggtttaacc

SEQ ID NO: 230 - DNA, 27 bp, Artificial Sequence
  Description of Artificial Sequence note = synthetic construct
atttgttccgagtcaaaacagcaagtc

SEQ ID NO: 231 - DNA, 27 bp, Artificial Sequence
  Description of Artificial Sequence note = synthetic construct
cggaactggtttcatctgattactttc

SEQ ID NO: 232 - DNA, 33 bp, Artificial Sequence
  Description of Artificial Sequence note = synthetic construct
ggtaaagctctgatatttgaagactctactgag

SEQ ID NO: 233 - DNA, 27 bp, Artificial Sequence
  Description of Artificial Sequence note = synthetic construct
aggcgatttaaaaaccaaggtcgatgt

SEQ ID NO: 234 - DNA, 28 bp, Artificial Sequence
  Description of Artificial Sequence note = synthetic construct
gtttctagatgaaggaagaaattgatcc

SEQ ID NO: 235 - DNA, 27 bp, Artificial Sequence
  Description of Artificial Sequence note = synthetic construct
atcgcatgcaagatatcgagtatgcgt

SEQ ID NO: 236 - DNA, 27 bp, Artificial Sequence
  Description of Artificial Sequence note = synthetic construct
ctcataattagcaagctgcctcagaat

SEQ ID NO: 237 - DNA, 27 bp, Artificial Sequence
  Description of Artificial Sequence note = synthetic construct
ccgtatgtggaatgtcgaactcatcgg

SEQ ID NO: 238 - DNA, 28 bp, Artificial Sequence
  Description of Artificial Sequence note = synthetic construct
agtatgtggaatgtcgaactcatcggct

SEQ ID NO: 239 - DNA, 23 bp, Artificial Sequence
  Description of Artificial Sequence note = synthetic construct
agggaaagtatgtggaatgtcct

SEQ ID NO: 240 - DNA, 33 bp, Artificial Sequence
  Description of Artificial Sequence note = synthetic construct
tgtggaatgtcgaactcatcggcgataagggtg

SEQ ID NO: 241 - DNA, 33 bp, Artificial Sequence; identical to SEQ ID NO: 232
  Description of Artificial Sequence note = synthetic construct

SEQ ID NO: 242 - DNA, 28 bp, Artificial Sequence
  Description of Artificial Sequence note = synthetic construct
cggtattagtatttgccgctttgagttc

SEQ ID NO: 243 - DNA, 27 bp, Artificial Sequence
  Description of Artificial Sequence note = synthetic construct
atcataaggcttggttcagcaggattc

SEQ ID NO: 244 - DNA, 26 bp, Artificial Sequence
  Description of Artificial Sequence note = synthetic construct
tcctccttgcaagctctgcctgtggg

SEQ ID NO: 245 - DNA, 48 bp, Artificial Sequence
  Description of Artificial Sequence note = synthetic construct
cctccttgcaagctctgcctgtggggttcctccttgcaagctctgcct

SEQ ID NO: 246 - DNA, 22 bp, Artificial Sequence
  Description of Artificial Sequence note = synthetic construct
acccgatataatccgtccttca

SEQ ID NO: 247 - DNA, 22 bp, Artificial Sequence
  Description of Artificial Sequence note = synthetic construct
cggctccttattcggtttaacc

SEQ ID NO: 248 - DNA, 18 bp, Artificial Sequence
  Description of Artificial Sequence note = synthetic construct
ttgaaacaccgcccggaa

SEQ ID NO: 249 - DNA, 23 bp, Artificial Sequence
  Description of Artificial Sequence note = synthetic construct
tttcggctccttattcggtttaa

SEQ ID NO: 250 - DNA, 27 bp, Artificial Sequence
  Description of Artificial Sequence note = synthetic construct
atttgttccgagtcaaaacagcaagtc

SEQ ID NO: 251 - DNA, 25 bp, Artificial Sequence
  Description of Artificial Sequence note = synthetic construct
cgtccttcaacatcagtgaaaatcg

SEQ ID NO: 252 - DNA, 27 bp, Artificial Sequence
  Description of Artificial Sequence note = synthetic construct
ccgcccttcaacatcagtgaaaatctt

SEQ ID NO: 253 - DNA, 27 bp, Artificial Sequence
  Description of Artificial Sequence note = synthetic construct
ctgatataatccgtccttcaacatcag

SEQ ID NO: 254 - DNA, 25 bp, Artificial Sequence
  Description of Artificial Sequence note = synthetic construct
cgcctatacgcctgctactttcacg

SEQ ID NO: 255 - DNA, 27 bp, Artificial Sequence
  Description of Artificial Sequence note = synthetic construct
cggaactggtttcatctgattactttc

SEQ ID NO: 256 - DNA, 19 bp, Artificial Sequence
  Description of Artificial Sequence note = synthetic construct
cctatacgcctgctacttt

SEQ ID NO: 257 - DNA, 80 bp, Artificial Sequence; identical to SEQ ID NO: 154
  Description of Artificial Sequence note = synthetic construct

SEQ ID NO: 258 - DNA, 33 bp, Artificial Sequence; identical to SEQ ID NO: 156
  Description of Artificial Sequence note = synthetic construct

SEQ ID NO: 259 - DNA, 28 bp, Artificial Sequence
  Description of Artificial Sequence note = synthetic construct
ttctaaaagtgactacaacttcctgcct

(19) United States
(12) Patent Application Publication
(10) Pub. No.: US 2015/0093756 A1
(43) Pub. Date: Apr. 2, 2015

(54) Thermophilic Helicase Dependent Amplification Technology With Endpoint Homogenous Fluorescent Detection

(71) Applicant: QIAGEN GAITHERSBURG, INC., GAITHERSBURG, MD (US)

(72) Inventors: John Wolff, Washington, DC (US); Victoria Doseeva, Rockville, MD (US); Thomas Forbes, Germantown, MD (US); Gwynne Roth, Germantown, MD (US); Irina Nazarenko, Gaithersburg, MD (US); Dirk Loeffert, Duesseldorf (DE)

(21) Appl. No.: 14/505,197

(22) Filed: Oct. 2, 2014

Related U.S. Application Data
(63) Continuation of application No. 12/695,001, filed on Jan. 27, 2010, now abandoned.
(60) Provisional application No. 61/147,623, filed on Jan. 27, 2009.
(60) Provisional application No. 61/180,821, filed on May 22, 2009.
(60) Provisional application No. 61/293,369, filed on Jan. 8, 2010.

(51) Int. Cl. C12Q 1/68

(52) U.S. Cl. CPC C12Q 1/686
USPC 435/6.12

(57) ABSTRACT

Disclosed herein are methods of amplifying a target nucleic acid in a helicase-dependent reaction. Also disclosed are methods of amplifying and detecting a target nucleic acid in a helicase-dependent reaction as well as modified detection labels to assist in the detection.

### CROSS-REFERENCE TO RELATED PATENT APPLICATIONS

This application claims priority to U.S. application Ser. No. 12/695,001, filed Jan. 27, 2010 and claims benefit of U.S. Provisional Application Nos. 61/147,623; 61/180,212; and 61/293,369, filed on Jan. 27, 2009, May 22, 2009 and Jan. 8, 2010, respectively which are all hereby incorporated herein by reference in their entirety.

### REFERENCE TO SEQUENCE LISTING

The Sequence Listing submitted Oct. 2, 2014 as a text file named “17107_{—}0001 U5_Sequence_Listing.txt,” created on Oct. 2, 2014, and having a size of 62,233 bytes is hereby incorporated by reference pursuant to 37 C.F.R. §1.52(e)(5).

### BACKGROUND OF THE INVENTION

Thermophilic Helicase Dependent Amplification (tHDA) is an isothermal amplification technology that utilizes helicase to unwind double-stranded DNA, removing the need for thermocycling. tHDA is a true isothermal DNA amplification method and has a simple reaction scheme, similar to PCR. The current tHDA, which employs UvrD helicase and Gst DNA polymerase, can achieve over a million-fold amplification. However, the performance of a tHDA system may be further improved as tHDA still has some major limitations: There is no established algorithm for primer design; primer-dimer formation is more pronounced in tHDA than in PCR; protection against amplicon carry-over is not yet developed; multiplexing is limited with UvrD tHDA system; tHDA is inefficient at amplifying long target sequences; and “hot start” tHDA currently is not available.

### SUMMARY OF THE INVENTION

Disclosed herein are methods of amplifying a target nucleic acid in a helicase-dependent reaction. Also disclosed are methods of amplifying and detecting a target nucleic acid in a helicase-dependent reaction as well as modified detection labels to assist in the detection.

The present invention provides a method amplifying a target nucleic acid in a helicase-dependent reaction, the method comprising:
(a) providing target nucleic acid to be amplified; wherein the target nucleic acid is double stranded and is denatured by heating at 65° C. for 10 minutes in the presence of 50 mM NaOH prior to step (b);
(b) adding oligonucleotide primers for hybridizing to the target nucleic acid of step (a);
(c) synthesizing an extension product of the oligonucleotide primers which are complementary to the templates, by means of a DNA polymerase to form a duplex;
(d) contacting the duplex of step (c) with a helicase preparation for unwinding the duplex such that the helicase preparation comprises a helicase and a single strand binding protein (SSB) unless the helicase preparation comprises a thermostable helicase wherein the single strand binding protein is optional; and
(e) repeating steps (b) (d) to exponentially and selectively amplify the target nucleic acid in a helicase-dependent reaction.

The present invention also provides the a method amplifying a target nucleic acid in a helicase-dependent reaction where the target nucleic acid is subjected to a “pre” step involving RNA probes and RNA-DNA hybrid capture antibodies. This method comprises:
(a) providing target nucleic acid to be amplified; wherein the target nucleic acid is single stranded DNA and wherein an RNA probes that is complementary is added to the single stranded DNA to bind to the DNA to form a target nucleic acid RNA-DNA hybrid; and wherein a hybrid capture antibodies that recognizes RNA-DNA hybrids bound to a magnetic bead is added to the RNA-DNA hybrid to be used in step (b)
(b) adding oligonucleotide primers for hybridizing to the target nucleic acid RNA-DNA hybrid of step (a);
(c) synthesizing an extension product of the oligonucleotide primers which are complementary to the templates, by means of a DNA polymerase to form a duplex;
(d) contacting the duplex of step (c) with a helicase preparation for unwinding the duplex such that the helicase preparation comprises a helicase and a single strand binding protein (SSB) unless the helicase preparation comprises a thermostable helicase wherein the single strand binding protein is optional; and
(e) repeating steps (b)-(d) to exponentially and selectively amplify the target nucleic acid in a helicase-dependent reaction.

The present invention also provides a modified TaqMan probe (and method using this probe). The probe has a short tail at the 3′- or 5′-end complementary to the 5′- or 3′-end, and wherein the TaqMan probe is complementary to the target nucleic acid except for this short tail, and wherein the short tail sequence forms a stem loop structure.

The present invention also provides modifications where certain additives are used to improve the assay. The additive is selected from the group consisting of DMSO, betaine, sorbitol, dextran sulfate and mixtures thereof.

Additional advantages of the disclosed methods and compositions will be set forth in part in the description which follows, and in part will be understood from the description, or can be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 provides the results of example 1a showing alkaline target denaturation in Ct/NG tHDA assay with Luminex detection.

FIG. 2 provides the results of example 1b showing alkaline target denaturation in NG1/NG2 tHDA assay with TaqMan probes in endpoint detection.

FIG. 3 shows the results of example 1c showing alkaline target denaturation in NG1/NG2 real-time tHDA assay with TaqMan probes.

FIG. 4 provides the results of example 2a of CT hybrid capture tHDA assay with Luminex detection.

FIG. 5 provides the results of example 2b.

FIG. 6 provides the results of example 2c—detection with EvaGreen Dye.

FIG. 7 provides the results of example 2c—detection with TaqMan probe.

FIG. 8 provides the results of example 4a—comparing effects of certain additives.

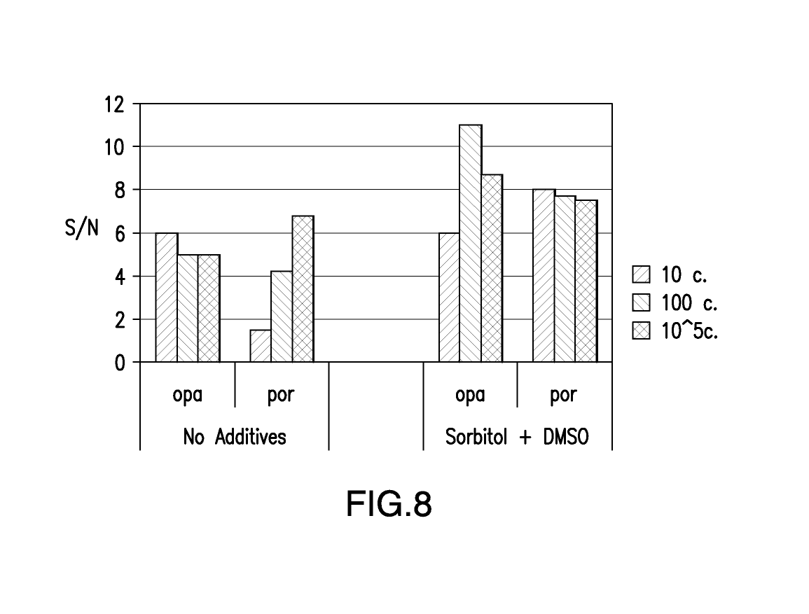

FIG. 9 provides the results of example 4b—comparing effects of certain additives.

FIG. 10 provides the results of example 4c—comparing effects of certain additives.

FIG. 11 provides anaylsis and confirmation of amplicon production by tHDA. The bar graph displays S/N data collected from a typical four target multiplex (4plex). Both CT amplicons have been optimized to have one fluorophore used for detection to simplify the assay.

FIG. 12 provides anaylsis and confirmation of amplicon production by tHDA. Melt curve analysis shows that all four amplicons are present.

FIG. 13 provides anaylsis and confirmation of amplicon production by tHDA. Gel analysis confirms the presence of desired amplified products.

FIG. 14: provides anaylsis and confirmation of amplicon production by tHDA. Realtime analysis of 4plex shows detections of four amplicons (two of which share the same fluorophore: green=internal control, blue=CT cryptic plasmid target/CT genomic target, red=NG taret)

FIG. 15 provides a diagram of HAD. A: Complementary DNA strands bound by SSB (orange circles) are shown as a thick top strand and thin lower strand are separated by helicase (blue circles) B: Hybridization of complimentary primers (black arrows) to the ssDNA template of the target region. C: Primers hybridized to the template DNA are extended by DNA polymerase (blue diamonds) D: Amplified products enter another cycle of amplification.

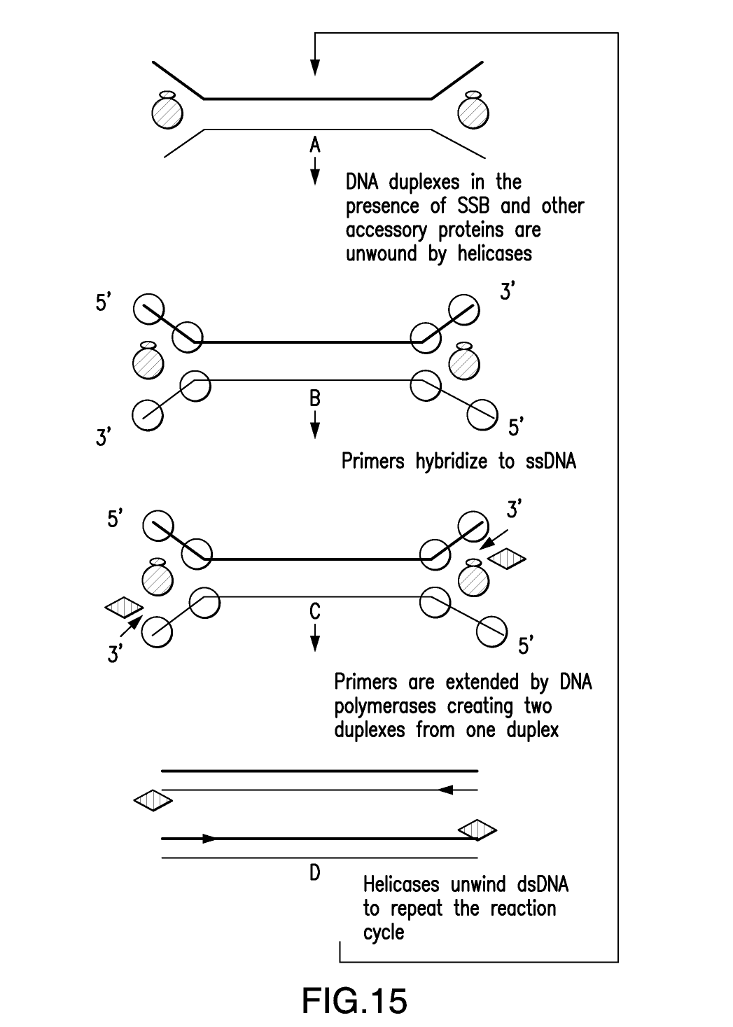

FIG. 16 provides sequences of some of the primers and probes used in the examples.

FIG. 17 shows a modified TaqMan probe (5′-CGTCCTTCAACATCAGTGAAAATC-3′ (SEQ ID NO. 132)) used to identify the presence of NG (5′-TTCTAAAAGTGACTACAACTTCCTGCCT-3′ (SEQ ID NO. 259)).

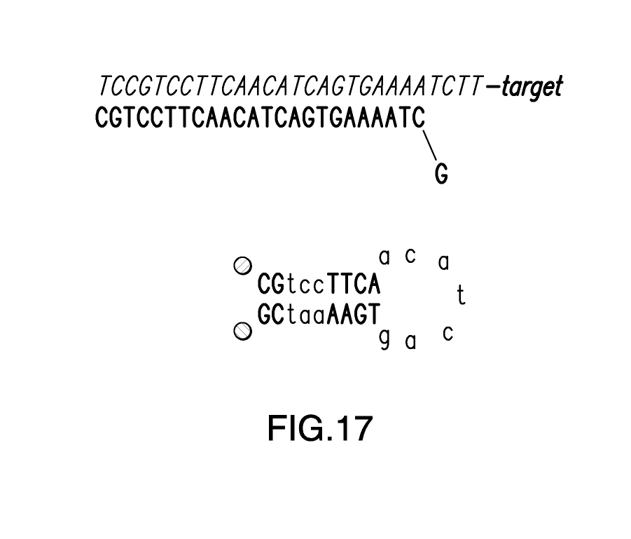

### DETAILED DESCRIPTION OF THE INVENTION

The present invention comprises methods and systems directed at determining the copy number of one or more target nucleic acids. The disclosed method and compositions can be understood more readily by reference to the following detailed description of particular embodiments and the Example included therein and to the Figures and their previous and following description.

All patents, patent applications and publications cited herein, whether supra or infra, are hereby incorporated by reference in their entireties into this application in order to more fully describe the state of the art as known to those skilled therein as of the date of the invention described and claimed herein. It is to be understood that this invention is not limited to specific synthetic methods, or to specific recombinant biotechnology methods unless otherwise specified, or to particular reagents unless otherwise specified.

Chlamydia trachomatis (CT) and Neisseria gonorrhoeae (NG) are currently the two most prevalent sexually transmitted infections reported in the US. While several diagnostic tests are currently available for the joint detection of CT and NG, some of which are PCR-based and therefore difficult to automate in a high throughput capacity.

Thermophilic helicase dependent amplification (tHDA) is a novel isothermal amplification technology allowing a simpler automation than PCR. tHDA utilizes helicase to unwind double-stranded DNA, thus removing the need for thermocycling. In conjunction with endpoint fluorescence detection, the tHDA isothermal reaction offers a potential alternative to PCR and real-time PCR for easily automatable diagnostic tests.

In part, described herein is a tHDA assay utilized to amplify selected target genes from both CT and NG. For CT amplification primers and dual-labeled fluorescent probes targeting regions of cryptic plasmid and genomic DNA sequences were designed. For NG, primers and probes specific for multicopy opa genes were used. For this aspect, endpoint fluorescence detection with dual-labeled probes was utilized for the detection of tHDA products. The detection was performed in a homogeneous format without opening the plate after amplification to avoid amplicon carry-over contamination.

Also disclosed herein is a multiplex tHDA CT/NG prototype assay allowing for simultaneous amplification and detection of NG and dual target genes from CT in the presence of an amplification control. The assay has achieved 10-25 copy sensitivity for both CT and NG pathogens.

As a result of the methods and examples described herein, tHDA, in conjunction with homogeneous endpoint fluorescence detection, provides a suitable technology platform for the development of a multi-target CT/NG detection assay, allowing high analytical sensitivity without the need for thermocycling equipment.

In another aspect, a method of amplifying and detecting C. trachomatis is described. In this method, tHDA amplification primers and Taqman probes targeting regions of cryptic plasmid and genomic DNA sequences were designed. For N. gonorrhoeae, primers and probes specific for multi-copy opa genes were used. In order to detect inhibition of the amplification reaction, an amplification inhibition control which utilizes CT primers for amplification was included in the assay. The tDHA assay is comprised of a 25 μl reaction that is run on a realtime detection platform for 120 minutes at 65° C. and then an endpoint fluorescence reading at 25° C.

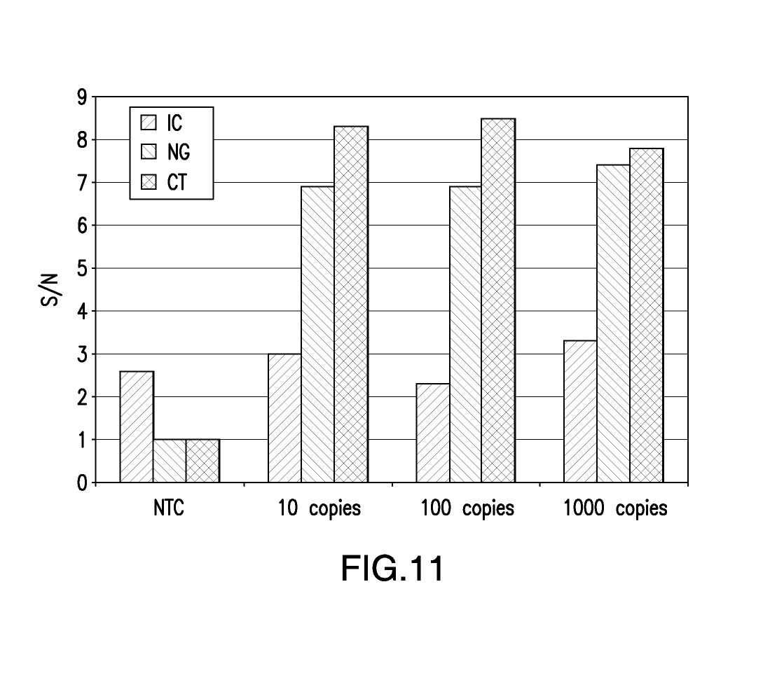

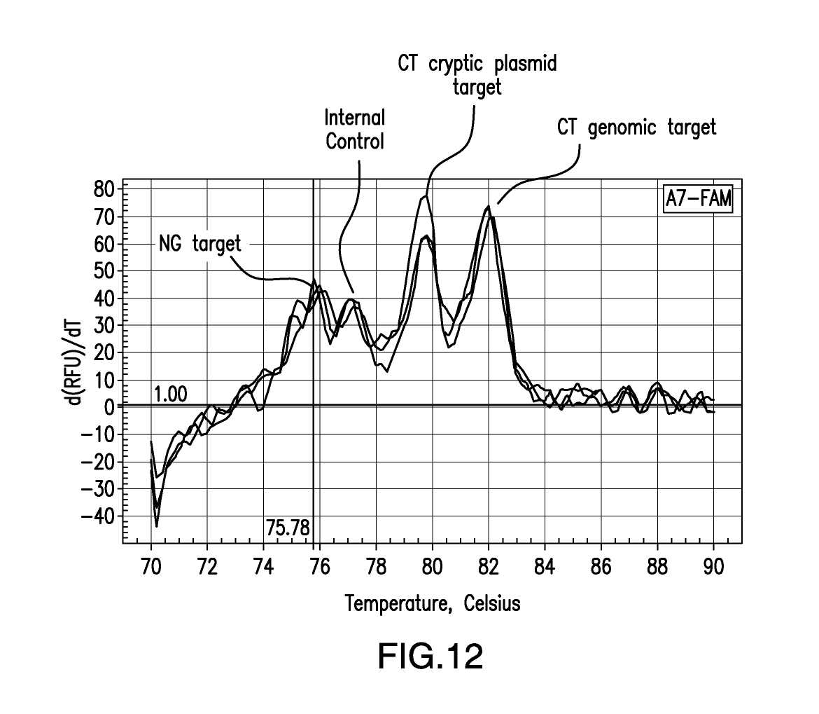

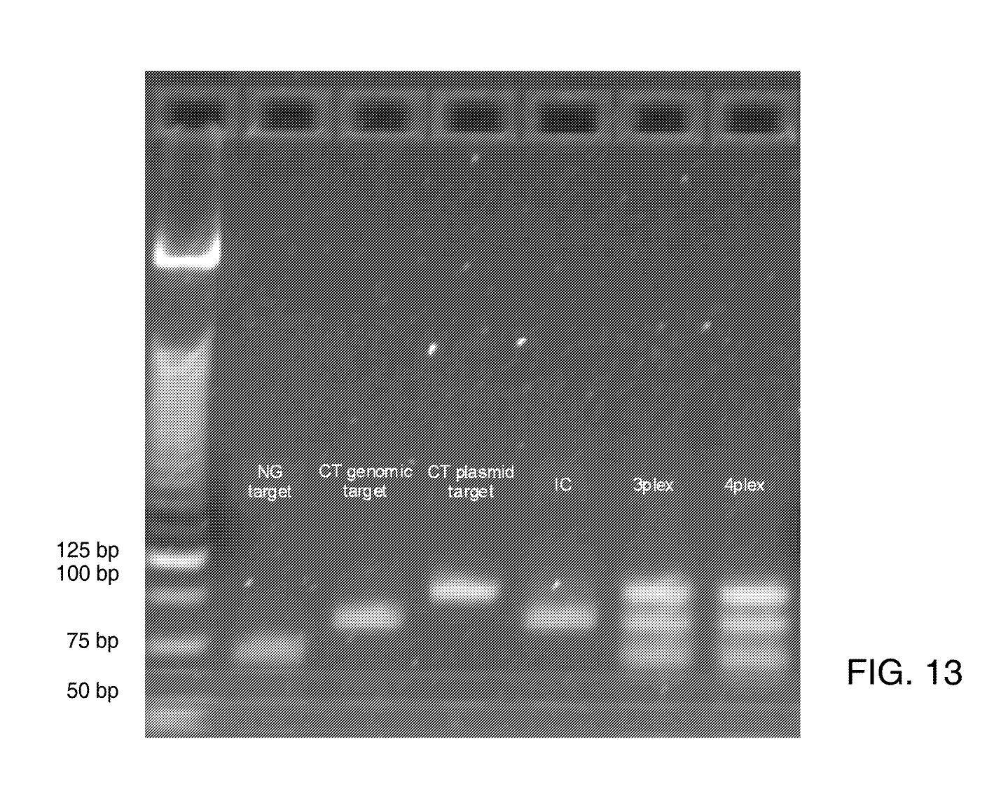

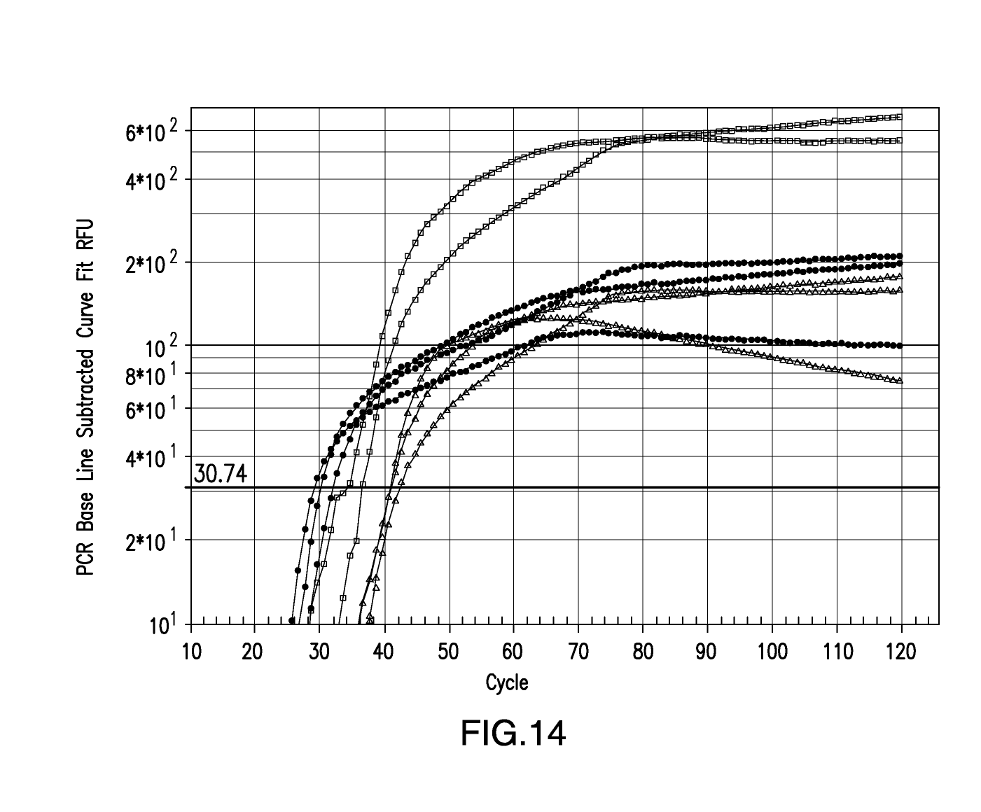

Also described herein are two multiplex tHDA CT/NG prototype assays, one of which has been optimized for use. Both prototype assays allow for simultaneous amplification and detection of N. gonorrhoeae and dual target genes from C. trachomatis in the presence of an amplification control. The assay duration for this aspect is approximately 120 minutes with additional time for endpoint detection and set-up leaving the total assay time to be <3 hours. The optimized isothermal multiplex assay has achieved a 10-25 copy level sensitivity for both pathogens with a S/N value >3 (FIG. 11). Real time data show targets are successfully amplified and detected (FIG. 12). Melting curve analysis shows four distinct peaks, one for each target amplicons (FIG. 13) and this result is further confirmed using a 4% agarose gel (FIG. 14).

Also described herein is are thermophilic helicase dependent amplification (“tHDA”) assays that can be used with multiple different detection technologies, including but not limited to Luminex's xMAP, real-time or endpoint fluorescence detection with TaqMan probes, melting curve analysis with Evagreen dye, or agarose gel electrophoresis. The methods described herein provide improvements on “Helicase Dependent Amplification” (HDA). HDA uses a helicase rather than heat to separate the two strands of a DNA duplex generating single-stranded templates for the purpose of in vitro amplification of a target nucleic acid. Sequence-specific primers hybridize to the templates and are then extended by DNA polymerases to amplify the target sequence. This process repeats itself so that exponential amplification can be achieved at a single temperature.

For example, described herein are methods wherein tHDA utilizes an alkaline denaturation step combined with heat to denature double stranded target nucleic acid before the tHDA. Target denaturation by NaOH at 65° C. was utilized to achieve 10-100 copies sensitivity for CT/NG tHDA assays. Chemical denaturation gives more consistent results than temperature denaturation (95° C.) for all targets, especially in a multiplex tHDA reaction. Alkali denaturation of the target improves performance of tHDA assay with dsDNA. (See example 1).

Also described herein are methods amplifying a target nucleic acid in a helicase-dependent reaction, the method comprising: (a) providing target nucleic acid to be amplified. When the target nucleic acid is double stranded, it is denatured by heating at 65° C. for 10 minutes in the presence of 50 mM NaOH prior to step (b). Step (b) involves adding oligonucleotide primers for hybridizing to the target nucleic acid of step (a). Step (c) is synthesizing an extension product of the oligonucleotide primers which are complementary to the templates, by means of a DNA polymerase to form a duplex. Then in step (d), the duplex of step (c) is contacted with a helicase preparation for unwinding the duplex. The helicase preparation comprises a helicase and a single strand binding protein (SSB), unless the helicase preparation comprises a thermostable helicase wherein the single strand binding protein is optional. Finally, steps (b)-(d) are repeated to exponentially and selectively amplify the target nucleic acid in a helicase-dependent reaction.

Also described herein are methods of amplifting a target nucleic acid from a biological sample. The biological sample containing a target nucleic acid (DNA) is subjected to a pre-treatment involving RNA probes and hybrid capture antibodies (antibodies that recognize RNA-DNA hybrids). A biological sample containing the target nucleic acid (DNA) is combined with RNA probes that are complementary and bind specifically to the target nucleic acid. When the RNA probes bind to the target nucleic acid, they form an RNA-DNA hybrid. Hybrid capture antibodies (antibodies that recognize RNA-DNA hybrids) that are bound to magnetic beads are then added to the sample containing the RNA-DNA hybrids. These beads are then washed to remove any unbound RNA-DNA hybrids. These beads can then be used directly in HDA amplification. The use of the hybrid capture sample preparation in the complete tHDA assay allows for the elimination of the target denaturation step. (See Example 2).

In some aspects, the tHDA assays can be used together with several detection methods, including but not limited to, Luminex (LMX) detection, Real-time and endpoint fluorescence detection with TaqMan probes, melting curve analysis with Evagreen dye, and agarose gel electrophoresis. Also described herein are modified TaqMan probes that can be used with the products of the tHDA assay in real time PCR detection. In this aspect, the completed tHDA assay is used and a modified TaqMan probe is added thereto for use in a real-time PCT reaction. The modified TaqMan probe has a short tail at the 3′- or 5′-end complementary to the 5′- or 3′-end. The modified TaqMan probe is complementary to the target nucleic acid except for this short tail, and the short tail sequence forms a stem loop structure. This modified TaqMan probe is different from molecular beacons, which form a stem-loop that does not contain any target sequence. TaqMan probes are linear probes labeled with a fluorophore and quencher. However, they often produce high fluorescent background because of incomplete quenching; which greatly decreases the signal-to-background ratio. The stem-loop hairpin structure of a modified TaqMan probe of the present invention can maximize quenching efficiency and minimum background signal. Therefore, signal to noise ratios for endpoint fluorescence detection of tHDA is greatly enhanced. (See Example 3).

Also described herein are methods and reagents that can be used to improve yield and specificity of difficult targets in tHDA amplifications by including enhancing agents in the reaction. Agents include: dimethyl sulfoxide (DMSO), N,N,N-trimethylglycine (betaine), sorbitol or dextran sulfate. DMSO was generally used at a final concentration of 1-2%. Betaine was generally used at a final concentration 0.1M-0.5M, Sorbitol was generally used at a final concentration of 0.1M-0.3M. Dextran Sulfate was generally used at a final concentration of 10 pM-1 nM. For some targets standard tHDA amplification conditions do not produce acceptable results. In those cases there are a number of additives that can be used to increase the yield and specificity of a reaction. Betaine and DMSO are two frequently used PCR additives that are effective separately or in combination. We have demonstrated their usefulness for increasing the efficiency and specificity of tHDA amplification as well. The use of Sorbitol in combination with DMSO also showed some beneficial effects on the performance of certain tHDA multiplexes. Adding sorbitol and DMSO to the tHDA reaction also helped to reduce non-specific amplification. DMSO functions by facilitating DNA strand separation. It is especially useful for GC rich templates. Betaine, as an isostabilizing agent, also acts on reducing secondary structure formation. Sorbitol acts as a protein stabilizer by displacing water molecules from the reaction. Therefore, sorbitol may protect the helicase and the polymerase against loss of activity during the amplification reaction. (See Example 4).

Also described herein are methods of amplifying a target nucleic acids in a helicase-dependent reaction. For example, disclosed herein are methods of amplifying a double stranded target nucleic acid comprising: (a) denaturing the target nucleic acid; (b) contacting one or more oligonucleotide probes with the denatured target nucleic acid, wherein the oligonucleotide probes hybridize to the denatured target nucleic acid to form double-stranded probe-target hybrids; (c) contacting the double-stranded probe-target hybrids with one or more capture antibodies wherein the one or more capture antibodies hybridize to the double-stranded probe-target hybrids to form captured double-stranded probe-target hybrids, (d) removing all uncaptured nucleic acids; (e) adding one or more oligonuceotide primers, wherein the oligonucleotide primers hybridize to the target nucleic acid; (f) synthesizing an extension product of the oligonucleotide primers which is complementary to the target nucleic acid, by means of a DNA polymerase to form a target nucleic acid duplex; and (g) contacting the target nucleic acid duplex of step (f) with a helicase preparation and amplifying the target nucleic acid duplex in a helicase-dependent reaction.

Also described herein is a method of amplifying a single stranded target nucleic acid in a helicase-dependent reaction, comprising: (a) contacting one or more oligonucleotide probes with the single stranded target nucleic acid, wherein the oligonucleotide probes hybridize to the target nucleic acid to form double-stranded probe-target hybrids; (b) contacting the double-stranded probe-target hybrids with one or more capture antibodies, wherein the capture antibodies hybridize to the double-stranded probe-target hybrids to form captured double-stranded probe-target hybrids, (c) removing all uncaptured nucleic acids; (d) adding one or more oligonuceotide primers, wherein the oligonucleotide primers hybridize to the target nucleic acid; (e) synthesizing an extension product of the oligonucleotide primers which is complementary to the target nucleic acid, by means of a DNA polymerase to form a target nucleic acid duplex; (f) contacting the target nucleic acid duplex of step (e) with a helicase preparation and amplifying the target nucleic acid duplex in a helicase-dependent reaction.

Also disclosed are methods of detecting the target nucleic acids amplified by the methods described herein.

### DEFINITIONS AND NOMENCLATURE

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a preparation” includes mixtures of compounds, and the like. Reference to “a component” can include a single or multiple components or a mixtures of components unless the context clearly dictates otherwise.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

The term “nucleic acid” refers to double stranded or single stranded DNA, RNA molecules or DNA/RNA hybrids. The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof. Those nucleic acids which are double stranded nucleic acid molecules may be nicked or intact. The double stranded or single stranded nucleic acid molecules may be linear or circular. The duplexes may be blunt ended or have single stranded tails. The single stranded molecules may have secondary structure in the form of hairpins or loops and stems. The nucleic acid may be isolated from a variety of sources including the environment, food, agriculture, fermentations, biological fluids such as blood, milk, cerebrospinal fluid, sputum, saliva, stool, lung aspirates, swabs of mucosal tissues or tissue samples or cells. Nucleic acid samples may obtained from cells or viruses and may include any of: chromosomal DNA, extra chromosomal DNA including plasmid DNA, recombinant DNA, DNA fragments, messenger RNA, transfer RNA, ribosomal RNA, double stranded RNA or other RNAs that occur in cells or viruses. Any of the above described nucleic acids may be subject to modification where individual nucleotides within the nucleic acid are chemically altered (for example, by methylation). Modifications may arise naturally or by in vitro synthesis.

The term “target nucleic acid” refers to a nucleic acid sought to be amplified, detected, or otherswise identified. In certain embodiments the target nucleic acid is Chlamydia trachomatis (“CT”) or Neisseria gonorrhoaea (“NG”) DNA or RNA.

The term “duplex” or “hybrid” refers to a nucleic acid molecule that is double stranded in whole or part. For example, a “double-stranded probe-target hybrid” refers to a nucleic acid molecule formed when an oligonucleotide probe hybridizes with a denatured target nucleic acid to form a double stranded nucleic acid molecule in the area whereby the oligonucleotide probe is specifically hybridized to the denatured target nucleic acid.

The terms “melting,” “unwinding” or “denaturing” refer to separating all or part of two complementary strands of a nucleic acid duplex or nucleic acid hybrid.

The terms “hybridization” or “hybridizes” is meant that the composition recognizes and physically interacts with another composition. For example, “hybridization” can refer to binding of an oligonucleotide primer to a region of a single-stranded nucleic acid template.

By “specifically binds” or “specifically hybridizes” is meant that the composition recognizes and physically interacts with its cognate target. For example, a primer can specifically bind to its target nucleic acid. For example, a primer specific to a sequence present in a cryptic plasmid can specifically hybridize to the cryptic plasmid and does not significantly recognize and interact with other targets or target nucleic acid sequences. The specificity of hybridization may be influenced by the length of the oligonucleotide primer, the temperature in which the hybridization reaction is performed, the ionic strength, and the pH.

By “probe,” “primer,” or “oligonucleotide” is meant a single-stranded DNA or RNA molecule of defined sequence that can base-pair to a second DNA or RNA molecule that contains a complementary sequence (the “target”). The term “primer” refers also to a single stranded nucleic acid capable of binding to a single stranded region on a target nucleic acid to facilitate polymerase dependent replication of the target nucleic acid. The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for target nucleic acids (for example, genes and/or mRNAs) have at least 80%-90% sequence complementarity, at least 91%-95% sequence complementarity, at least 96%-99% sequence complementarity, or at least 100% sequence complementarity to the region of the target to which they hybridize. Probes, primers, and oligonucleotides may be detectably-labeled, either radioactively, or non-radioactively, by methods well-known to those skilled in the art. Probes or oligonucleotide probes can be used for methods involving nucleic acid hybridization, such as: the described methods of forming double-stranded probe-target hybrids between an oligonucleotide probe and a denatured target nucleic acid. Primers and oligonucleotide primers can be used for methods involving nucleic acid hybridization, such as: synthesizing an extension product of an oligonucleotide primer hybridized to a target nucleic acid, which is complementary to the target nucleic acid or for amplifying a target nucleic acid in a tHDA reaction. Probes, primers and oligonucleotides can also be used for nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, and electrophoretic mobility shift assay (EMSA).

By “primer set” is meant to mean at least two primers that each contain a complementary sequence to an opposite strand of the same target sequence. In a primer set, at least one of the two primers must be a “forward primer” at least one of the two primers must be a “reverse primer”. A “forward primer” is a primer that is complementary to a sense strand of a target nucleic acid, wherein a “reverse primer” is a primer that is complementary to the complement of the sense strand of the target nucleic acid (also referred to as the anti-sense strand of the target nucleic acid). A primer set can be a pair of primers capable of being used in a tHDA reaction.

Similarly, by “oligonucleotide probe” is meant to mean a single-stranded DNA or RNA molecule of defined sequence that can base-pair to a second DNA or RNA molecule that contains a complementary sequence. In accordance with the present invention, one or more oligonucleotide probes are contacted with a denatured nucleic acid sequence under conditions sufficient for the one or more polynucleotide probes to hybridize to the denatured target nucleic acid form double-stranded probe-target hybrids. In some aspects, the target nucleic acid is DNA and the oligonucleotide probes are RNA.

By “amplicon” is meant to mean pieces of DNA formed as the products of natural or artificial amplification events. For example, they can be formed via the methods described herein, tHDA, polymerase chain reactions (PCR) or ligase chain reactions (LCR), as well as by natural gene duplication.

By “specifically hybridizes” is meant that a probe, primer, or oligonucleotide recognizes and physically interacts (that is, base-pairs) with a substantially complementary nucleic acid (for example, a target nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The term “accessory protein,” refers to any protein capable of stimulating helicase activity may be used. For example, E. coli MutL protein is an accessory protein (Yamaguchi et al. J. Biol. Chem. 273:9197 9201 (1998); Mechanic et al., J. Biol. Chem. 275:38337 38346 (2000)) for enhancing UvrD helicase melting activity. In embodiments of the method, accessory proteins can be used with selected helicases. In alternative embodiments, unwinding of nucleic acids may be achieved by helicases in the absence of accessory proteins.

In certain embodiments a “cofactor” maybe used. A “cofactor” refers to small-molecule agents that are required for the helicase unwinding activity. Helicase cofactors include nucleoside triphosphate (NTP) and deoxynucleoside triphosphate (dNTP) and magnesium (or other divalent cations). For example, ATP (adenosine triphosphate) may be used as a cofactor for UvrD helicase at a concentration in the range of 0.1 100 mM and preferably in the range of 1 to 10 mM (for example 3 mM). Similarly, dTTP (deoxythymidine triphosphate) may be used as a cofactor for T7 Gp4B helicase in the range of 1 10 mM (for example 3 mM).

The term “HDA” refers to Helicase Dependent Amplification which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification.

“Thermophilic Helicase Dependent Amplification” or “tHDA” refers to an isothermal amplification technology that utilizes helicase to unwind double-stranded DNA, removing the need for thermocycling. tHDA is a true isothermal DNA amplification method and has a simple reaction scheme, similar to PCR. Basic, tHDA is described in U.S. Pat. No. 7,282,328 (Kong et al.) an is hereby incorporated by reference in its entirety.

The term “isothermal amplification” refers to amplification which occurs at a single temperature. This does not include the single brief time period (less than 15 minutes) at the initiation of amplification which may be conducted at the same temperature as the amplification procedure or at a higher temperature.

The term “helicase preparation” refers to a mixture of reagents that when combined with a DNA polymerase, a nucleic acid template, four deoxynucleotide triphosphates, and oligonucleotide primers are capable of achieving isothermal, specific nucleic acid amplification in vitro.

The term “oligonucleotide probe” refers to a single-stranded DNA or RNA molecule of defined sequence that can base-pair to a second DNA or RNA molecule that contains a complementary sequence. In accordance with the methods described herein, one or more oligonucleotide probes are contacted with a denatured nucleic acid sequence under conditions sufficient for the one or more polynucleotide probes to hybridize to the denatured target nucleic acid form double-stranded probe-target hybrids.

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W.H. Freeman and Company (2nd ed. (1992)), especially chapter 11).

The term “detection label” refers to any molecule that can be associated with amplified target nucleic acid, directly or indirectly, and which results in a measurable, detectable signal, either directly or indirectly.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an oligonucleotide probe is disclosed and discussed and a number of modifications that can be made to a number of molecules including the oligonucleotide probe are discussed, each and every combination and permutation of the oligonucleotide probe and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Compositions for Hybrid Capture

1. Target Nucleic Acids

The disclosed compositions are designed to interact either directly or indirectly with target nucleic acids. A “target nucleic acid” can be any nucleic acid sought to be amplified, detected, or otherswise identified. In general, any natural nucleic acid, synthetic nucleic acid, modified nucleic acid or nucleic acid derivative can be a target nucleic acid. A target nucleic acid can include, without limitation, DNA, RNA, mRNA, viral RNA, ribosomal RNA cDNA, gDNA, ssDNA, dsDNA or any combination thereof. For example, in certain aspects, the target nucleic acid is Chlamydia trachomatis (“CT”) or Neisseria gonorrhoaea (“NG”) DNA.

In addition, a target nucleic acid can be single or double-stranded. A target nucleic acid can be isolated from a variety of sources including the environment, food, agriculture, fermentations, biological fluids such as urine, blood, milk, cerebrospinal fluid, sputum, saliva, stool, lung aspirates, swabs of mucosal tissues or tissue samples or cells. Any of the above described target nucleic acids may be subject to modification where individual nucleotides within the nucleic acid are chemically altered (for example, by methylation). Modifications may arise naturally or by in vitro synthesis.

The disclosed methods can be used to amplify, detect or identify target nucleic acids. The disclosed methods can also be used to amplify, detect or identify differences between target nucleic acids or differences from a control nucleic acid. Target nucleic acids can also be associated directly or indirectly with substrates, preferably in arrays.

As used herein, unless the context indicates otherwise, the term target nucleic acids refers to both actual nucleic acids and to nucleic acid sequences that are part of a larger nucleic acid molecule.

2. Target Samples

Samples that contain or that may contain target nucleic acids can be referred to as target samples. Target nucleic acid samples may obtained from cells or viruses and may include any of: chromosomal DNA, extra chromosomal DNA including plasmid DNA, recombinant DNA, DNA fragments, messenger RNA, transfer RNA, ribosomal RNA, double stranded RNA or other RNAs that occur in cells or viruses.

Target samples can be derived from any source that has, or is suspected of having, target nucleic acids. A target sample can be the source of target nucleic acids. Target samples can contain, for example, a target nucleic acid such as DNA or RNA. A target sample can include natural target nucleic acids, chemically synthesized target nucleic acids, or both. A target sample can be, for example, a sample from one or more cells, tissue, or bodily fluids such as blood, urine, semen, lymphatic fluid, cerebrospinal fluid, or amniotic fluid, or other biological samples, such as tissue culture cells, buccal swabs, nasal swabs, sputum, mouthwash, stool, tissues slices, biopsy aspiration, and archeological samples such as bone or mummified tissue. Types of useful target samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

Target nucleic acid samples can be derived from any source that has, or is suspected of having, target nucleic acids. A target nucleic acid sample is the source of target nucleic acid molecules and target nucleic acid sequences. Target nucleic acid sample can contain, for example, a target nucleic acid, for example a specific mRNA or pool of mRNA molecules, a specific DNA, or a specific viral RNA. The target nucleic acid sample can contain RNA or DNA or both. The target nucleic acid sample in certain aspects can also include chemically synthesized target nucleic acids. The target nucleic acid sample can include any nucleotide, nucleotide analog, nucleotide substitute or nucleotide conjugate.

3. Oligonucleotide Probes

An “oligonucleotide probe” refers to a single-stranded DNA or RNA molecule of defined sequence that can base-pair to a second DNA or RNA molecule that contains a complementary sequence. In accordance with the present invention, one or more oligonucleotide probes are contacted with a denatured nucleic acid sequence under conditions sufficient for the one or more polynucleotide probes to hybridize to the denatured target nucleic acid form double-stranded probe-target hybrids. In some aspects, the target nucleic acid is DNA and the oligonucleotide probes are RNA. The oligonucleotide probes can be between 15 and 100 nucleotides. For example, the oligonucleotide probes can be between 20 and 30 nucleotides long.

In some aspects, the RNA oligonucleotide probes are short oligonucleotide probes as opposed to full length transcribed RNA oligonucleotide probes. These short RNA oligonucleotide probes can also be referred to herein as synthetic RNA oligonucleotide probes or “synRNA.” In some aspects, the target nucleic acid is RNA and the oligonucleotide probes are DNA.

In aspects, one or more oligonucleotide probes are used (i.e. more than one probe). The one or more oligonucleotide probes can be specific for one or more target nucleic acids. For example, if there are two target nucleic acids to be amplified or detected, oligonucleotide probes capable of specifically hybridizing to each, but not both, of the target nucleic acids can be used. For example, both CT and NG can be amplified in the same reaction using one or more oligonucleotide probes specific to CT and one or more oligonucleotide probes specific to NG.

In some aspects, one or more oligonucleotide probes can be used to ensure coverage of about 3-4 kb of a target nucleic acid, which ensures a strong, readable signal. In some aspects, amplification or detection of CT, using the methods described herein, can employ one or more of the following oligonucleotide probes listed in Table 1.

TABLE 1

| | SEQ |
|---|---|
| RNA oligonucleotide probes | ID NO. |
| GCTGCTCGAACTTGTTTAGTACCTTCGGTCCAAGAAGTCTT | 1 |
| GGCAGAGGA | |
| AACTTTTTTAATCGCATCTAGAATTAGATTATGATTTAAAA | 2 |
| GGGAAAACT | |
| CTTGCAGATTCATATCCAAGGACAATAGACCAATCTTTTCT | 3 |
| AAAGACAAA | |
| AAAGATCCTCGATATGATCTACAAGTATGTTTGTTGAGTGA | 4 |
| TGCGGTCCA | |
| ATGCATAATAACTTCGAATAAGGAGAAGCTTTTCATGCGTT | 5 |
| TCCAATAGG | |
| ATTCTTGGCGAATTTTTAAAACTTCCTGATAAGACTTTTCG | 6 |
| CTATATTCT | |
| AACGACATTTCTTGCTGCAAAGATAAAATCCCTTTACCCAT | 7 |
| GAAATCCCT | |
| CGTGATATAACCTATCCGTAAAATGTCCTGATTAGTGAAAT | 8 |
| AATCAGGTT | |
| GTTAACAGGATAGCACGCTCGGTATTTTTTTATATAAACAT | 9 |
| GAAAACTCG | |
| TTCCGAAATAGAAAATCGCATGCAAGATATCGAGTATGCGT | 10 |
| TGTTAGGTA | |
| AAGCTCTGATATTTGAAGACTCTACTGAGTATATTCTGAGG | 11 |
| CAGCTTGCT | |
| AATTATGAGTTTAAGTGTTCTCATCATAAAAACATATTCAT | 12 |
| AGTATTTAA | |
| ATACTTAAAAGACAATGGATTACCTATAACTGTAGACTCGG | 13 |
| CTTGGGAAG | |
| AGCTTTTGCGGCGTCGTATCAAAGATATGGACAAATCGTAT | 14 |
| CTCGGGTTA | |
| ATGTTGCATGATGCTTTATCAAATGACAAGCTTAGATCCGT | 15 |
| TTCTCATAC | |
| GGTTTTCCTCGATGATTTGAGCGTGTGTAGCGCTGAAGAAA | 16 |
| ATTTGAGTA | |
| ATTTCATTTTCCGCTCGTTTAATGAGTACAATGAAAATCCA | 17 |
| TTGCGTAGA | |
| TCTCCGTTTCTATTGCTTGAGCGTATAAAGGGAAGGCTTGA | 18 |
| CAGTGCTAT | |
| AGCAAAGACTTTTTCTATTCGCAGCGCTAGAGGCCGGTCTA | 19 |
| TTTATGATA | |
| TATTCTCACAGTCAGAAATTGGAGTGCTGGCTCGTATAAAA | 20 |
| AAAAGACGA | |

In some aspects, amplification or detection of CT, using the methods described herein, can employ one or more of the following oligonucleotide probes listed in Table 2.

TABLE 2

| Oligonnuceotide | | |
|---|---|---|
| Probe Names | Oligonucleotide Probe Sequences | SEQ ID NO. |
| OMP probes | | |
| Omp3 | TCCTCCTTGCAAGCTCTGCCTGTGGG | 21 |
| Omp4 | TTCCTCCTTGCAAGCTCTGCCTGTGGGAGGAA | 22 |
| Omp6 | CTTCCTCCTTGCAAGCTCTGCCTGTGGGAGGAAG | 23 |
| Omp7 | CCTCCTTGCAAGCTCTGCCTGTGGGG | 24 |
| Omp8 | TTCCTCCTTGCAAGCTCTGCCT | 25 |
| CT F9R6 probes: | | |
| p5 F9R6 | AGTATGTGGAATGTCGAACTCATCGGC T | 26 |
| p6 F9R6 | CCGTATGTGGAATGTCGAACTCATCGG | 27 |
| p2 F9R6 | GTGATAGGGAAAGTATGTGGAATGTC | 28 |
| CTp48 | AGGGAAAGTATGTGGAATGTCCT | 29 |
| CTp49 | AAAGTATGTGGAATGTCGAACTCTTT | 30 |
| Other cryptic | | |
| plasmid CT probes: | | |
| CTp23 | ACGTGCGGGCGATTTGCCTTAACCCCACC | 31 |
| CTp26 | CGTGCGGGCGATTTGCCTTAACCCCACCGCACG | 32 |
| CTp39 | AACGTGCGGGCGATTTGCCTTAACCCCACCGCACG | 33 |
| CTp40 | AACGTGCGGGCGATTTGCCTT | 34 |
| CTp34 | TGGCGAATTTTTAAAACTTCCTGATAAGACTTTTCGC | 35 |
| CTp35 | GCGAATTTTTAAAACTTCCTGATAAGACTTTTCGC | 36 |
| p6 | CCGTATGTGGAATGTCGAACTCATCGG | 37 |
| CT plasmid probes: | | |
| CTplas25-1 | CUAGCGGUAAAACUGCUUACUGGUC | 38 |
| CTplas25-2 | AGAUAAAAUCCAUACAGAAGCAACA | 39 |
| CTplas25-3 | CGUACUUCUUUUAGGAGAAAAAAUC | 40 |
| CTplas25-4 | UAUAAUGCUAGAAAAAUCCUGAGUA | 41 |
| CTplas25-5 | AGGAUCACUUCUCCUCAACAACUUU | 42 |
| CTplas25-6 | UUCAUCUUGGAUAGAGUUAGUUUUU | 43 |
| CTplas25-7 | AGAACUAAGUCUUCUGCUUACAAUG | 44 |
| CTplas25-8 | CUCUUGCAUAUUACGAGCUUUUUAU | 45 |
| CTplas25-9 | AAACCUCCCCAACCAAACUCUACAA | 46 |
| CTplas25-10 | AAAGAGUUUCAAUCGAUCCCCUAUA | 47 |
| CTplas25-11 | AAUCCGCAUAUAUUUUGGCCGCUAG | 48 |
| CTplas25-12 | GACGUUAGAGAAACGAUAGAUAAGU | 49 |
| CTplas25-13 | CUGAUUCAGAGAAGAAUCGCCAAUU | 50 |
| CTplas25-14 | AUCUGAUUUCUUAAUAGAGAUACUU | 51 |
| CTplas25-15 | CGCAUCAUGUGUUCCGGAGUUUCUU | 52 |
| CTplas25-16 | UGUCCUCCUAUAACGAAAAUCUUCU | 53 |
| CTplas25-17 | ACAACAGCUUUUUGAACUUUUUAAG | 54 |
| CTplas25-18 | CAAAAGAGCUGAUCCUCCGUCAGCU | 55 |
| CTplas25-19 | CAUAUAUAUAUCUAUUAUAUAUAUA | 56 |
| CTplas25-20 | UAUUUAGGGAUUUGAUUUUACGAGA | 57 |
| CT genome probes: | | |
| CTgeno25-1 | AAGGGCUUCUUCCUGGGACGAACGU | 58 |
| CTgeno25-2 | UUUUCUUAUCUUCUUUACGAGAAUA | 59 |
| CTgeno25-3 | AGAAAAUUUUGUUAUGGCUCGAGCA | 60 |
| CTgeno25-4 | UUGAACGACAUGUUCUCGAUUAAGG | 61 |
| CTgeno25-5 | CUGCUUUUACUUGCAAGACAUUCCU | 62 |
| CTgeno25-6 | CAGGCCAUUAAUUGCUACAGGACAU | 63 |
| CTgeno25-7 | CUUGUCUGGCUUUAACUAGGACGCA | 64 |
| CTgeno25-8 | GUGCCGCCAGAAAAAGAUAGCGAGC | 65 |
| CTgeno25-9 | ACAAAGAGAGCUAAUUAUACAAUUU | 66 |
| CTgeno25-10 | AGAGGUAAGAAUGAAAAAACUCUUG | 67 |
| CTgeno25-11 | CGGAAUUCUAUGGGAAGGUUUCGGC | 68 |
| CTgeno25-12 | GGAGAUCCUUGCGAUCCUUGCACCA | 69 |
| CTgeno25-13 | CUUGGUGUGACGCUAUCAGCAUGCG | 70 |
| CTgeno25-14 | UAUGGGUUACUAUGGUGACUUUGUU | 71 |
| CTgeno25-15 | UUCGACCGUGUUUUGCAAACAGAUG | 72 |
| CTgeno25-16 | UGAAUAAAGAAUUCCAAAUGGGUGC | 73 |
| CTgeno25-17 | CAAGCCUACAACUGCUACAGGCAAU | 74 |
| CTgeno25-18 | GCUGCAGCUCCAUCCACUUGUACAG | 75 |
| CTgeno25-19 | CAAGAGAGAAUCCUGCUUACGGCCG | 76 |
| CTgeno25-20 | ACAUAUGCAGGAUGCUGAGAUGUUU | 77 |

In some aspects, amplification or detection of NG, using the methods described herein, can employ one or more of the following oligonucleotide probes listed in Table 3.

In some aspects, amplification or detection of NG, using the methods described herein, can employ one or more of the following oligonucleotide probes listed in Table 4.

In some aspects, internal control sequence can also be amplified or detected, using the methods described herein, can employ one or more of the following oligonucleotide probes listed in Table 5.

In some aspects an oligonucleotide probe mixture comprising multiple sets of probes is used to simultaneously screen for any one or more of a mixture of desired target nucleic acids. For example, it may be desirable to screen a biological sample for the presence of NG and CT in the same sample. In such a situation, a probe mixture of some, and in some cases, all of the probes provided in Tables 1-5 are used. For example, a probe mixture can be designed to provide a probe set for CT, NG as well as an internal control. Furthermore, multiple oligonucleotide probes can be used to hybridize to different regions of the same target sequence.

The oligonucleotide probes described herein enable sensitive detection of a one or more target nucleic acid sequence, while also achieving excellent specificity against even very similar related target nucleic acid sequences.

The one or more oligonucleotide probes can be designed so that they do not hybridize to a variant of the target nucleic acid or to non-target nucleic acid sequences under the hybridization conditions utilized. The number of different oligonucleotide probes employed per set can depend on the desired sensitivity. Higher coverage of the nucleic acid target with the corresponding oligonucleotide probes can provide a stronger signal (as there will be more DNA-RNA hybrids for the capture antibodies to bind).

TABLE 3

| | SEQ |
|---|---|
| RNA oligonucleotide probes | ID NO. |
| ACCGATATAGGGTTTGAATTTGTCGTTGAG | 78 |
| TTTGAAATCGTAAACGGCGGACAAGCCGAG | 79 |
| AGAAGAAACGGCGTGGAACGTACCGTTTTC | 80 |
| CTGATTTTCCGCCTTCAGATATTGCGTCAC | 81 |
| GTTTATCTTTTCGCCCTTGTTTTCGTTCAC | 82 |
| CTTTTTTGTGTTGACGGAATATTTACTGTT | 83 |
| GTTCCACTTTCTGTAACGGGCATAATCTGC | 84 |
| CGCTATCCTCCAGCCGCCGAAGTCGTAGCC | 85 |
| GACCGACACCCTGGGGTGGATGGAATGCGT | 86 |
| ACGGATGTTTCTGAAATAATCGCTTACCGT | 87 |
| GCTTATTTTGTCTTTTTTTGTACCGGTTGG | 88 |
| TTCCGGATAATCGTGGGTAATGCGTTCGGC | 89 |
| GGCGTAGGCTAAATCCGCCTGCACATACGG | 90 |
| GCCGCGGCCATTGCCTTCACTTGCCGCCTG | 91 |
| CGCTGCGGAAGAGAAGAGAAGGTTTTTTGC | 92 |
| GGGCTGGATTCATTTTCGGCTCCTTATTCG- | 93 |
| GTTTAACCGGTTAAAAAAAAGATTTTCACT | 94 |
| GATGTTGAAGGGCGGATTATATCGGGTTCC- | 95 |
| GGGCGGTGTTTCAACACAATATGGCGGATG | 96 |
| AACAAAAACCGGTACGGGTTGCCCCGCCCC | 97 |
| GGCTCAAAGGGAACGGTTCCCTAAGACGCC | 98 |
| CAAGCACCGGGCGGATCGGTTCCGTACCAT | 99 |
| TTGTACCGTCTGCGGCCCGCCGCCTTGTCC | 100 |
| TGATTTTTGTTAATCCGCTATACGTCTGAT | 101 |
| TGATGCCGAATCTTTGGAAGAAGTCTTGAA | 102 |
| ACAATAGAAGCAGGCAATTGGAATAGGGTT | 103 |
| TTCTTTTCATAAGAAACAGCCGCAAAGACC | 104 |
| GTGATCTTTGCGGCTGTCTGTTTTCTGTCC | 105 |
| GTCAGAACCGGTAGCCTACGCCGATTTGTC | 106 |
| CGCTGTGGTTGCCGTACTGTTTGGAACCGG | 107 |
| TGTAGCTGTAACGTGCCAAGCCGTTCCAGC | 108 |
| CGGCAACCCGGCGGGTGTGCGGCATATTGC | 109 |
| GTGCACCCGTCTTGCCGGTTGCTGCAGCCG | 110 |
| CGTTGCCGAATTCGACATCCACCCCCAGAC | 111 |

TABLE 4

| Oligonucloetide probes | Sequence | SEQ ID NO. |
|---|---|---|
| PorA5 probes | | |
| porA5 GCp5 FAM | TCCGCCTATACGCCTGCTACTTTCACGCTG | 112 |
| porA5_VD1 FAM | TCCGCCTATACGCCTGCTACTTTCACGCTGG | 113 |
| porA5_VD2_FAM | TCCGCCTATACGCCTGCTACTTTCACGCTGGA | 114 |
| porA5_VD3_FAM | CCTATACGCCTGCTACTTTCACGGG | 115 |
| porA5_VD4_FAM | CCTATACGCCTGCTACTTTCACGAGG | 116 |
| porA5_VD6_FAM | CCTATACGCCTGCTACTTTCACGCTG | 117 |
| porA5_VD7_FAM | CCATATACGCCTGCTACTTTCACGTGG | 118 |
| porA_probe_FAM | CGTGAAAGTAGCAGGCGTATAGGCGGACTT | 119 |
| porA7 probes: | | |
| porA7_p1 | CGCAGTCAGAAACGCGAACATACC | 120 |
| porA7_p2 | CAGTCAGAAACGCGAACATACCAGCTG | 121 |
| porA7_p3 | AACGCAGTCAGAAACGCGAACATACC | 122 |
| Other por probes: | | |
| PROBE 940_1005 | GCGAGTGATACCGATCCAT | 123 |
| PorA probe | | |
| porA10_p2 probe | CGAGGAAGCCGATATGCGACTCG | 124 |
| PROBE 4 PorA | CGCCTATACGCCTGCTACTT | 125 |
| PROBE 3 PorA | GCCTGCTACTTTCACGCTG | 126 |
| opaK_Probe_2 LMX | CCGCCCTTCAACATCAGTGAAAATCTT | 127 |
| opaD 3′ Probe LMX | CCGCCCTTCAACATCAGT | 128 |
| opaD b2 | TCCGTCCTTCAACATCAGTGAAAATCGGA | 129 |
| OpaDp1 MGB | CGTCCTTCAACATCAGTGAAAAT | 130 |
| opaD b3 | CTGATATAATCCGTCCTTCAACATCAG | 131 |
| opaD b1 | CGTCCTTCAACATCAGTGAAAATCG | 132 |
| porA5_VD5 | CGCCTATACGCCTGCTACTTTCACG | 133 |
| Additional | | |
| probes for NG | | |
| NGopa25-1 | CUGCAGAUGCCCGACGGUCUUUAUA | 134 |
| NGopa25-2 | GCGGAUUAACAAAAAUCAGGACAAG | 135 |
| NGopa25-3 | GGGCGGGCCGCAGGCAGUACAAAUG | 136 |
| NGopa25-4 | GUACGGAACCGAUCCGCCCGGUGCU | 137 |
| NGopa25-5 | UGGGCGCCUUAGGGAACCGUUCCCU | 138 |
| NGopa25-6 | UUGAGCCGGGGCGGGGCAACGACGU | 139 |
| NGopa25-7 | ACCGGUUUUUGUUCAUCCGCCAUAU | 140 |
| NGopa25-8 | CCAGCCCCCAAAAAACCUUCUCUUC | 141 |
| NGopa25-9 | UCUUCUCUUCUCUUCUCUUCUCUUC | 142 |
| NGopa25-10 | UCUUCCGCAGCGCAGGCGGCGGGUG | 143 |
| NGopa25-11 | AAGACCAUGGCCGCGGCCCGUAUGU | 144 |
| NGopa25-12 | GCAGGCGGAUUUAGCCUACGCCUAC | 145 |
| NGopa25-13 | GAACACAUUACCCACGAUUAUCCGG | 146 |
| NGopa25-14 | AACAAACCGCUCCAAAAAAAGCACA | 147 |
| NGopa25-15 | AUUAAGCACGGUAAGCGAUUAUUUC | 148 |
| NGopa25-16 | AGAAACAUCCGUACGCAUUCCAUCC | 149 |
| NGopa25-17 | ACCCCAGGGUGUCGGUCGGCUACGA | 150 |
| NGopa25-18 | CUUCGGCGGCUGGAGGAUAGCGGCA | 151 |
| NGopa25-19 | GAUUAUGCCCGUUACAGAAAGUGGA | 152 |
| NGopa25-20 | ACAACAAUAAAUAUUCCGUUAACAU | 153 |

TABLE 5

| Oligonucloetide probes | Sequence | SEQ ID NO. |
|---|---|---|
| Internal controls | | |
| sequences and IC probes | | |
| GIC1 | GTATTTGCCGCTTTGAGTTCATAACGTCCGGCG | 154 |
| | AGTTGTCTCATCCACCACCGGAAAAAAGAATC | |
| | CTGCTGAACCAAGCC/3C6/ | |
| CTp42 | AACGTCCGGCGAGTTGTCTCAT | 155 |
| CTp36 | CGTCCGGCGAGTTGTCTCATCCACCACCGGACG | 156 |
| IC-CT ompF5R4 | CGGTATTAGTATTTGCCGCTTTGAGTTCTGATC | 157 |
| | GAGAGCTCATATGACCACGGCCGGCTGAATCC | |
| | TGCTGAACCAAGCCTTATGAT | |
| IC-CT | CGGTATTAGTATTTGCCGCTTTGAGTACTGATC | 158 |
| ompF5R4_2MM | GAGAGCTCATATGACCACGGCCGGCTGTATCCT | |
| | GCTGAACCAAGCCTTATGAT | |
| IC probe1_FAM | CGAGAGCTCATATGACCACG | 159 |
| IComp p1 | ATCGAGAGCTCATATGACCACGGCCGAT | 160 |
| IComp p3 | ATCGAGAGCTCATATGACCACGAT | 161 |
| IComp p5 | GATCGAGAGCTCATATGACCACGATC | 162 |
| IC-F9R17_4MM | AGGCGATTTAAAAACCAAGGTCGTTCTTGATCG | 163 |
| | AGAGCTCATATGACCACGGCCGGCTCCATTAG | |
| | GGTGTTGGATCAATTTCTTC | |
| IC-F9R17_2MM | AGGCGATTTAAAAACCAAGGTCGATCTTGATC | 164 |
| | GAGAGCTCATATGACCACGGCCGGCTCCATAA | |
| | GGGTGTTGGATCAATTTCTTC | |
| Additional IC Probes: | | |
| ICbs25-1 | GCCCGGUACCCAGCUUUUGUUCCCU | 165 |
| ICbs25-2 | UUAGUGAGGGUUAAUUGCGCGCUUG | 166 |
| ICbs25-3 | GCGUAAUCAUGGUCAUAGCUGUUUC | 167 |
| ICbs25-4 | CUGUGUGAAAUUGUUAUCCGCUCAC | 168 |
| ICbs25-5 | AAUUCCACACAACAUACGAGCCGGG | 169 |
| ICbs25-6 | AGCAUAAAGUGUAAAGCCUGGGGUG | 170 |
| ICbs25-7 | CCUAAUGAGUGAGCUAACUCACAUU | 171 |
| ICbs25-8 | AAUUGCGUUGCGCUCACUGCCCGCU | 172 |
| ICbs25-9 | UUCCAGUCGGGAAACCUGUCGUGCC | 173 |
| ICbs25-10 | AGCUGCAUUAAUGAAUCGGCCAACG | 174 |
| ICbs25-11 | ACGCUGCGCGUAACCACCACACCCG | 175 |
| ICbs25-12 | CCGCGCUUAAUGCGCCGCUACAGGG | 176 |
| ICbs25-13 | CGCGUCCCAUUCGCCAUUCAGGCUG | 177 |
| ICbs25-14 | CGCAACUGUUGGGAAGGGCGAUCGG | 178 |
| ICbs25-15 | UGCGGGCCUCUUCGCUAUUACGCCA | 179 |
| ICbs25-16 | GCUGGCGAAAGGGGGAUGUGCUGCA | 180 |
| ICbs25-17 | AGGCGAUUAAGUUGGGUAACGCCAG | 181 |
| ICbs25-18 | GGUUUUCCCAGUCACGACGUUGUAA | 182 |
| ICbs25-19 | AACGACGGCCAGUGAGCGCGCGUAA | 183 |
| ICbs25-20 | UACGACUCACUAUAGGGCGAAUUGG | 184 |

One method of determining the one or more polynucleotide probes can be found in U.S. patent application Ser. No. 12/426,076, which is specifically incorporated by reference in its entirety and especially for its teaching of oligonucleotide probes and methods of using and identifying the same. For example, depending on the target nucleic acid of interest, and the corresponding non-target nucleic acids, the one or more polynucleotide probes can be prepared to have lengths sufficient to provide target-specific hybridization to the sought after target nucleic acid sequence.

The one or more polynucleotide probes can each have a length of at least about 15 nucleotides, illustratively, about 15 to about 1000, about 20 to about 800, about 30 to about 400, about 40 to about 200, about 50 to about 100, about 20 to about 60, about 20 to about 40, about 20 to about 20 and about 25 to about 30 nucleotides. In some aspects, the one or more polynucleotide probes each have a length of about 25 to about 50 nucleotides. In some aspects, the probes have a length of 25 nucleotides. In some aspects, all of the probes in a set will have the same length, such as 25 nucleotides, and will have very similar melting temperatures to allow hybridization of all of the probes in the set under the same hybridization conditions.

Bioinformatics tools can also be employed to determine the one or more oligonucleotide probes. For example, Oligoarray 2.0, a software program that designs specific oligonucleotides can be utilized. Oligoarray 2.0 is described by Rouillard et al. Nucleic Acids Research, 31: 3057-3062 (2003), which is incorporated herein by reference. Oligoarray 2.0 is a program which combines the functionality of BLAST (Basic Local Alignment Search Tool) and Mfold (Genetics Computer Group, Madison, Wis.). BLAST, which implements the statistical matching theory by Karlin and Altschul (Proc. Natl. Acad. Sci. USA 87:2264 (1990); Proc. Natl. Acad. Sci. USA 90:5873 (1993), is a widely used program for rapidly detecting nucleotide sequences that match a given query sequence One of ordinary skill in the art can provide a database of sequences, which are to be checked against, for example presence or absence of CT or NG. The target sequence of interest, e.g. the outer membrane protein gene for CT, can then be BLASTed against that database to search for any regions of identity. Melting temperature (Tm) and % GC can then be computed for one or more polynucleotide probes of a specified length and compared to the parameters, after which secondary structure also can be examined Once all parameters of interest are satisfied, cross hybridization can be checked with the Mfold package, using the similarity determined by BLAST. The various programs can be adapted to determine the one or more polynucleotide probes meeting the desired specificity requirements. For example, the parameters of the program can be set to prepare polynucleotides of 25 nt length, Tm range of 55-95° C., a GC range of 35-65%, and no secondary structure or cross-hybridization at 55° C. or below.

4. Double Stranded Probe Target Hybrids

The term “double-stranded probe-target hybrid” refers to the double stranded molecule formed from contacting one or more oligonucleotide probes with a single stranded target nucleic acid (either originally single stranded or denatured to become single stranded), wherein the oligonucleotide probes hybridize to the denatured target nucleic acid. For example, a double-stranded probe-target hybrid can be comprised of a oligonucleotide probe hybridized to a target nucleic acid. A a double-stranded probe-target hybrid can serve as a target for one or more capture antibodies.

5. Capture Antibodies

Capture antibodies can also be used in the methods described herein. Capture antibodies can be used to enrich a reaction for the target nucleic acid sequence. For example, in some aspects of the described methods double-stranded probe-target hybrids are contacted with one or more capture antibodies wherein the one or more capture antibodies hybridize to the double-stranded probe-target hybrids to form captured double-stranded probe-target hybrids. As used herein, the term “hybrid capture antibody” refers to antibodies capable of specifically binding to RNA-DNA hybrids. For example, the term “capture antibody” can refer to an antibody that is immunospecific to double-stranded nucleic acid hybrids.

In the disclosed methods double-stranded probe-target hybrids formed in accordance with the described methods can be captured with one or more capture antibodies that are immunospecific to double-stranded nucleic acid hybrids. Capture antibodies can be immunospecific to double-stranded hybrids, including, but not limited to, RNA/DNA; DNA/DNA; RNA/RNA; and mimics thereof, where “mimics” as defined herein, refers to molecules that behave similarly to RNA/DNA, DNA/DNA, or RNA/RNA hybrids. The capture antibody used will depend on the type of double-stranded nucleic acid hybrid formed. In one aspect, the capture antibody is immunospecific to RNA/DNA hybrids.

It will be understood by those skilled in the art that either polyclonal or monoclonal capture antibodies can be used and/or immobilized on a solid support or phase in the present assay as described below. Monoclonal antibody prepared using standard techniques can be used in place of the polyclonal antibodies. Also included are immunofragments or derivatives of capture antibodies, where such fragments or derivatives contain binding regions of the capture antibody.

For example, a polyclonal RNA:DNA specific antibody derived from goats immunized with an RNA:DNA hybrid can be used. Capture antibodies can be purified from the goat serum by affinity purification against RNA:DNA hybrid immobilized on a solid support, for example as described in Kitawaga et al., Mol. Immunology, 19:413 (1982); and U.S. Pat. No. 4,732,847, each of which is incorporated herein by reference.

Other suitable methods of producing or isolating antibodies, including human or artificial antibodies, can be used, including, for example, methods which select recombinant antibody (e.g. single chain Fv or Fab, or other fragments thereof) from a library, or which rely upon immunization of transgenic animals (e.g., mice) capable of producing a repertoire of human antibodies (see, e.g. Jakobovits et al. Proc. Natl. Acad. Sci. USA, 90:2551 (1993); Jakobovits et al., Nature, 362: 255 (1993); and U.S. Pat. Nos. 5,545,806 and 5,545,807).

In one aspect, the target nucleic acid to be determined is DNA (e.g., NG genomic DNA) or RNA (e.g., mRNA, ribosomal RNA, nucleolar RNA, transfer RNA, viral RNA, heterogeneous nuclear RNA), wherein the one or more oligonucleotide probes are polyribonucleotides or polydeoxyribonucleotides, respectively. According to this aspect, the double-stranded nucleic acid hybrids (i.e. double-stranded probe-target hybrids that are DNA/RNA hybrids) formed can be captured using a capture antibody that is immunospecific to RNA:DNA hybrids.

While any vertebrate may be used for the preparation of monoclonal anti-RNA/DNA capture antibodies, goats or rabbits are preferred. Preferably, a goat or rabbit is immunized with a synthetic poly(A)-poly(dT) hybrid by injecting the hybrid into the animal in accordance with conventional injection procedures. Polyclonal capture antibodies may be collected and purified from the blood of the animal with antibodies specific for the species of the immunized animal in accordance with well-known antibody isolation techniques. For the production of monoclonal capture antibodies, the spleen can be removed from the animal after a sufficient amount of time, and splenocytes can be fused with the appropriate myeloma cells to produce hybridomas. Hybridomas can then be screened for the ability to secrete the anti-hybrid antibody. Selected hybridomas may then be used for injection into the peritoneal cavity of a second animal for production of ascites fluid, which may be extracted and used as an enriched source of the desired monoclonal antibodies incorporated herein by reference.

The capture antibody can also be biotinylated and subsequently immobilized on, for example streptavidin coated tubes or silica, or modified by other methods to covalently bind to the solid phase. Solubilized biotinylated capture antibodies can be immobilized on a streptavidin coated tubes before capture of the double-stranded probe-target hybrids.

In aspects, double-stranded probe-target hybrids are incubated in tubes coated with one or more capture antibodies for a sufficient amount of time to allow capture of the double-stranded probe-target hybrids by the immobilized capture antibodies. The double-stranded probe-target hybrids can be bound to the immobilized capture antibodies by incubation, for example incubation for about 5 minutes to about 24 hours at about 15 to about 65° C. In some embodiments, the incubation time is about 30 to about 120 minutes at about 20 to about 40° C., with shaking at about 300 to about 1200 rpm. In another embodiment, capture occurs with incubation at about one hour at about room temperature with vigorous shaking on a rotary platform. It will be understood by those skilled in the art that the incubation time, temperature, and/or shaking can be varied to achieve alternative capture kinetics as desired.

In other aspects, the capture antibody can be coupled to a magnetic bead (e.g., COOH-beads). Magnetic bead-based technology is well known in the art. In some aspects, magnetic silica beads having derivatized surfaces for reacting with the capture antibody can be employed. For example, when the RNA oligonucleotide probes bind to a DNA target nucleic acid, they form an RNA-DNA hybrid, hybrid capture antibodies (antibodies that recognize RNA-DNA hybrids) that are bound to magnetic beads can then be added to the sample containing the RNA-DNA hybrids. Once the capture antibodies hybridize to the double-stranded probe-target hybrids, they form captured double-stranded probe-target hybrids

In another aspect, a capture antibody as described above can be conjugated to a detection label. Conjugation methods for labeling are well known in the art. For example, a capture antibody can be conjugated to a detectable label such as alkaline phosphatase. It will be understood by those skilled in the art that any detectable label such as an enzyme, a fluorescent molecule, or a biotin-avidin conjugate can be used. The antibody conjugate can be produced by well known methods such as direct reduction of the monoclonal antibody with dithiothreitol (DTT) to yield monovalent antibody fragments. The reduced antibody can then be directly conjugated to maleimated alkaline phosphatase by the methods of Ishikawa et al., J. Immunoassay 4:209-237 (1983) and Means et al., Chem. 1: 2-12 (1990), and the resulting conjugate can be purified by HPLC.

Thus, target-specific oligoribonucleotides or oligodeoxynucleotides can be designed using commercially available bioinformatics software. For example, for the detection of dsDNA targets, DNA can be denatured, hybridized to the RNA probes, and captured via anti-RNA:DNA hybrid antibodies on a solid support. Detection can be performed by various methods, including anti-RNA:DNA capture antibodies conjugated with alkaline phosphatase for chemiluminescent detection. Alternatively, other detection methods can be employed, for example using anti-RNA:DNA capture antibodies conjugated with phycoerythrin, suitable for detection by fluorescence.

6. Captured Double Stranded Probe Target Hybrids

As described elsewhere herein, the methods comprise, in part, hybridizing one or more oligonucleotide probes to a target nucleic acid (denatured in the case where the target nucleic acid is double-stranded), to form double-stranded probe-target hybrids. Once the double-stranded probe-target hybrids are formed, hybrid capture antibodies conjugated to solid support (for example paramagnetic beads) (antibodies that recognize double-stranded nucleic acid hybrids) can bindto the double-stranded probe-target hybrids As such, “double-stranded probe-target hybrids” refer to a composition comprising the target nucleic acid sequence, and capture probes, where the target nucleic acid sequence and oligonucleotide probes are hybridized to one another (i.e. double-stranded probe-target hybrid) and the capture antibody is bound to the double-stranded probe-target hybrid. For example, in some aspects the methods comprise, in part, hybridizing one or more RNA oligonucleotide probes to a DNA target nucleic acid to form an RNA-DNA hybrid, hybrid capture antibodies (antibodies that recognize RNA-DNA hybrids) that are bound to magnetic beads can then be added to the sample containing the RNA-DNA hybrids. Once the capture antibodies hybridize to the double-stranded probe-target hybrids, they form captured double-stranded probe-target hybrids.

Once captured double-stranded probe-target hybrids are formed, they can be immobilized as described above or by other methods well known in the art. Once immobilized, non-captured double-stranded probe-target hybrids can be removed from the reaction by washing away any non-captured, non-immobilized materials, such as non-target nucleic acids, cellular debris, etc. Solutions to be used for washes are known in the art and one of skill in the art would understand how to perform the described washes. For example, any buffer that does not hydrolyze target and capture probes and does not denature the antibodies can be used.

For example, reactions can then be washed with a wash buffer (e.g. 0.1 M Tris-HCl, pH 7.5, 0.6 M NaCl, 0.25% Tween-20™, and sodium azide) to remove as much of the non-captured double-stranded probe-target hybrids or non-specifically bound double-stranded probe-target hybrids as possible.

B. Compositions for tHDA

1. Oligonucleotide Primers

As described above “HDA” refers to Helicase Dependent Amplification which is an in vitro method for amplifying nucleic acids by using a helicase preparation for unwinding a double stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse-complementary target sequence. The HDA reaction is a general method for helicase-dependent nucleic acid amplification. Oligonucleotide primers can also be used to synthesize an extension product of the oligonucleotide primers which is complementary to the target nucleic acid to which it is hybridized.

In the methods described herein, oligonucleotide primers suitable for use include, but are not limited to an oligonucleotide or oligomer having a sequence complementary to one or more portions of a target nucleic acid sequence or complement thereof. Oligonucleotide primers can also include modified nucleotides to make it resistant to exonuclease digestion. For example, the oligonucleoctide primer can have phosphorothioate linkages between one or more nucleotides An oligonuceotide primer is specific for, or corresponds to, a target nucleic acid sequence or the complement thereof. A complementary portion is not substantially complementary to another sequence if it has a melting temperature 10° C. lower than the melting temperature under the same conditions of a sequence fully complementary to the complementary portion of the target.

Generally, primer pairs suitable for use in HDA are short synthetic oligonucleotides, for example, having a length of more than 10 nucleotides and less than 50 nucleotides. Oligonucleotide primer design involves various parameters such as string-based alignment scores, melting temperature, primer length and GC content (Kampke et al., Bioinformatics 17:214 225 (2003)). When designing a primer, one of the important factors is to choose a sequence within the target fragment which is specific to the nucleic acid molecule to be amplified. The other important factor is to decide the melting temperature of a primer for HDA reaction. The melting temperature of a primer is determined by the length and GC content of that oligonucleotide. In some aspects, the melting temperature of a primer can be about 10 to 30° C. higher than the temperature at which the hybridization and amplification will take place. For example, if the temperature of the hybridization and amplification is set at 37° C. when using the E. coli UvrD helicase preparation, the melting temperature of a pair of primers designed for this reaction should be in a range between about 47° C. to 67° C. If the temperature of the hybridization and amplification is 60° C., the melting temperature of a pair of primers designed for that reaction can be in a range between 65° C. and 90° C. To choose the best primer for a HDA reaction, a set of primers with various melting temperatures can be tested in a parallel assays. More information regarding primer design is described by Kampke et al., Bioinformatics 17:214 225 (2003).

Each oligonuceotide primer in an HAD reaction hybridizes to each end of the target nucleic acid and may be extended in a 3′ to 5′ direction by a polymerase using the target nucleotide sequence as a template. Conditions of hybridization are standard as described in “Molecular Cloning and Laboratory Manual” 2.sup.nd ed. Sambrook, Rich and Maniatis, pub. Cold Spring Harbor (2003). To achieve specific amplification, a homologous or perfect match primer is preferred. However, primers may include sequences at the 5′ end which are non complementary to the target nucleotide sequence(s). Alternatively, primers may contain nucleotides or sequences throughout that are not exactly complementary to the target nucleic acid. Primers may represent analogous primers or may be non-specific or universal primers for use in HDA as long as specific hybridization can be achieved by the primer-template binding at a predetermined temperature.

The primers may include any of the deoxyribonucleotide bases A, T, G or C and/or one or more ribonucleotide bases, A, C, U, G and/or one or more modified nucleotide (deoxyribonucleotide or ribonucleotide) wherein the modification does not prevent hybridization of the primer to the nucleic acid or elongation of the primer or denaturation of double stranded molecules. Primers may be modified with chemical groups such as phosphorothioates or methylphosphonates or with non nucleotide linkers to enhance their performance or to facilitate the characterization of amplification products.

To detect amplified target nucleic acids, the primers can be subjected to modification, such as fluorescent or chemiluminescent-labeling, and biotinylation. (for example, fluorescent tags such as amine reactive fluorescein ester of carboxyfluorescein-Glen Research, Sterling, Va.). Other labeling methods include radioactive isotopes, chromophores and ligands such as biotin or haptens which while not directly detectable can be readily detected by reaction with labeled forms of their specific binding partners, for example, avidin and antibodies respectively.

Oligonucleotide primers as described herein can be prepared by methods known in the art (see, for example U.S. Pat. No. 6,214,587).

In one aspect, a pair of two sequence-specific primers, one hybridizing to the 5′-border of the target sequence and the other hybridizing to the 3′-border of the target are used in HDA to achieve exponential amplification of a target sequence. This approach can be readily distinguished from Lee et al. (J. Mol. Biol. 316:19 34 (2002)). Multiple pairs of primers can be utilized in a single HDA reaction for amplifying multiple targets simultaneously using different detection tags in a multiplex reaction. Multiplexing is commonly used in SNP analysis and in detecting pathogens (Jessing et al., J. Clin. Microbiol. 41:4095 4100 (2003)).

Also disclosed herein are oligonucleotide primers that can be used to amplify Chlamydia trachomatis (CT) or Neisseria gonorrhoeae (NG). For example, disclosed are primers that can be used to amplify the multi-copy Opa gene, the cryptic plasmid genomic DNA, and the outer membrane protein (OMP) gene.

Disclosed herein are primers that can be used to amplify Chlamydia trachomatis. Such primers include the primers listed in Table 6.

TABLE 6

| Oligonucleotide | Oligonucleotide | SEQ |
|---|---|---|
| Primer Name | Primer Sequence | ID NO. |
| ORF 3F | ATCGCATGCAAGATATCGAGTATGCGT | 185 |
| ORF 3R | CTCATAATTAGCAAGCTGCCTCAGAAT | 186 |
| OmpF3 | AGTATTTGCCGCTTTGAGTTCTGCTTC | 187 |
| OmpR3 | GATCATAAGGCTTGGTTCAGCAGGATT | 188 |
| CT ORF Forw | ATCGCATGCAAGATATCGAGTATGCGT | 189 |
| CT ORF Rev | CTCATAATTAGCAAGCTGCCTCAGAAT | 190 |
| CT F12 | AACCAAGGTCGATGTGATAGGGAAAGT | 191 |
| CTR10 | TCGTTTCTCTAACGTCTTTGTTTCTAGATG | 192 |
| CT F11 | AAAACCAAGGTCGATGTGATAGGGAAA | 193 |
| CT R9 | TCTCTAACGTCTTTGTTTCTAGATGAAGG | 194 |
| Forw: CT 1296CGG | CGGGGTTATCTTAAAAGGGATTGCAGCTTG | 195 |
| Rev: CT 1410 | TCAACGAAGAGGTTTTGTCTTCGTAAC | 196 |
| Forw: CT 2013 | GCTTTTCATGCGTTTCCAATAGG | 197 |
| Rev: CT 2107 | CTTTGCAGCAAGAAATGTCGTTAG | 198 |
| Omp F5 | CGGTATTAGTATTTGCCGCTTTGAGTTC | 199 |
| Omp R4 | ATCATAAGGCTTGGTTCAGCAGGATTC | 200 |
| omp F13 | ATTTGCCGCTTTGAGTTCTGCTTCCT | 201 |
| omp R4 | ATCATAAGGCTTGGTTCAGCAGGATTC | 202 |
| F9 | AGGCGATTTAAAAACCAAGGTCGATGT | 203 |
| R17 | GAAGAAATTGATCCAACACCCTTATCG | 204 |

Disclosed herein are primers that can be used to amplify Neisseria gonorrhoeae. Such primers include the primers listed in Table 7.

TABLE 7

| Oligonucleotide | Oligonucleotide | SEQ |
|---|---|---|
| Primer Name | Primer Sequence | ID NO. |
| PorA3 F | TGTTCCGAGTCAAAACAGCAAGTC | 205 |
| PorA3 R | GCCGGAACTGGTTTCATCTGATTA | 206 |
| PorAF4 | AATTTGTTCCGAGTCAAAACAGCAAGT | 207 |
| PorAR4 | GGAACTGGTTTCATCTGATTACTTTCC | 208 |
| PorA F6 | AGCCACCCTCAGAAGGTCAAAC | 209 |
| PorA R6 | AACGAGCCGAAATCACTGACTTT | 210 |
| PorA F7 | CTATGCCCATGGTTTCGACTTTGT | 211 |
| PorA R7 | GTAATCGACACCGGCGATGA | 212 |
| PorA F8 | TGCCCATGGTTTCGACTTTG | 213 |
| PorA R8 | GTAATCGACACCGGCGATGAT | 214 |
| PorA F10 | AATTGGAGACTGATTGGGTGTTTG | 215 |
| PorA R10 | AATACGAGGGCGGTAAGTTTTTTT | 216 |
| PorA F11 | CGGCTCAGTTGGATTTGTCTGA | 217 |
| PorA R11 | GATGCGCGGGACTGTATTACC | 218 |
| GC porA 940F | TTCTTTTTGTTCTTGCTCGGCAGA | 219 |
| GCporA 1005R | GCGGTGTACCTGATGGTTTTT | 220 |
| opaD_For | TTGAAACACCGCCCGGAA | 221 |
| opaD_Rev | TTTCGGCTCCTTATTCGGTTTAA | 222 |
| opaDv F7 | GTTCATCCGCCATATTGTGTTG | 223 |
| opaDv R7 | CACTGATGTTGAAGGACGGATTAT | 224 |
| opaDv R4 | TTCGGCTCCTTATTCGGTTTAAC | 225 |
| OpaK F1 | CCGATATAATCCGCCCTTC | 226 |
| OpaK R1 | TTCGGCTCCTTATTCGGTTT | 227 |
| opaDv F1_6 | ACCCGATATAATCCGTCCTTCA | 228 |
| opaDv R1 | CGGCTCCTTATTCGGTTTAACC | 229 |
| PorA F5 | ATTTGTTCCGAGTCAAAACAGCAAGTC | 230 |
| PorA R5 | CGGAACTGGTTTCATCTGATTACTTTC | 231 |

2. DNA Polymerases

Polymerases can be selected for the methods described herein based on the basis of processivity and strand displacement activity as well as the temperatures used in the particular method being employed. For example, polymerases for tHDA can be selected on the basis of processivity and strand displacement activity. Subsequent to melting and hybridization with an oligonucleotide primer, the nucleic acid can be subjected to a polymerization step. Examples of polymerases include, but are not limited to DNA polymerases. DNA polymerases for use in the disclosed compositions and methods can also be highly processive, if desired. A DNA polymerase is selected if the nucleic acid to be amplified is DNA. The suitability of a DNA polymerase for use in the disclosed compositions and methods can be readily determined by assessing its ability to carry out strand elongation or tHDA.

When the initial target is RNA, a reverse transcriptase can be used first to copy the RNA target into a cDNA molecule and the cDNA is then further amplified in tHDA by a selected DNA polymerase. The DNA polymerase acts on the target nucleic acid to extend the hybridized oligonucleotide primers hybridized to the nucleic acid templates in the presence of four dNTPs to form primer extension products complementary to the nucleotide sequence on the nucleic acid template.

In addition, a polymerase capable of carrying out the Reverse transcription reaction as well as DNA polymerase activity in the tHDA reaction can be used in the methods described herein. For example. HIV-1 reverse transcriptase from human immunodeficiency virus type 1 (PDB 1HMV), M-MLV reverse transcriptase from the Moloney murine leukemia virus, or AMV reverse transcriptase from the avian myeloblastosis virus can be used alone or in combination.

The DNA polymerases for the methods described herein can be selected from a group of DNA polymerases lacking 5′ to 3′ exonuclease activity and which additionally may lack 3′-5′ exonuclease activity.

Examples of suitable DNA polymerases include an exonuclease-deficient Klenow fragment of E. coli DNA polymerase I (New England Biolabs, Inc. (Beverly, Mass.)), an exonuclease deficient T7 DNA polymerase (Sequenase; USB, (Cleveland, Ohio)), Klenow fragment of E. coli DNA polymerase I (New England Biolabs, Inc. (Beverly, Mass.)), Large fragment of Bst DNA polymerase (New England Biolabs, Inc. (Beverly, Mass.)), KlenTaq DNA polymerase (AB Peptides, (St Louis, Mo.)), T5 DNA polymerase (U.S. Pat. No. 5,716,819), and Pol III DNA polymerase (U.S. Pat. No. 6,555,349). DNA polymerases possessing strand-displacement activity, such as the exonuclease-deficient Klenow fragment of E. coli DNA polymerase I, Bst DNA polymerase Large fragment, and Sequenase, can be used for Helicase-Dependent Amplification. T7 polymerase is a high fidelity polymerase having an error rate of 3.5.times.10⁵ which is significantly less than Taq polymerase (Keohavong and Thilly, Proc. Natl. Acad. Sci. USA 86, 9253 9257 (1989)). T7 polymerase is not thermostable however and therefore is not optimal for use in amplification systems that require thermocycling. In HDA, which can be conducted isothermally, T7 Sequenase can be used for amplification of DNA.

3. Target Nucleic Acid Duplex

A “target nucleic acid duplex” refers to a double stranded nucleic acid, comprising, in part a target nucleic acid sequence, a complement of a target nucleic acid sequence, or a copy thereof A target nucleic acid duplex can be created by synthesizing an extension product of an oligonucleotide primer which is complementary to the target nucleic acid to which the oligonucleotide primer is hybridized, by means of a DNA polymerase. A target nucleic acid duplex can serve as a template for HDA or tHDA. For example, a target nucleic acid duplex can be contacted with a helicase and polymerase preparation to amplify the target nucleic acid duplex in a helicase-dependent reaction.

4. Helicase Preparations

In the methods described herein, the helicase can be provided in a “helicase preparation.” The “helicase preparation” refers to a mixture of reagents that when combined with a DNA polymerase, a nucleic acid template, four deoxynucleotide triphosphates, and oligonucleotide primers are capable of achieving isothermal, specific nucleic acid amplification in vitro.

More particularly, the helicase preparation can include a helicase, an energy source such as a nucleotide triphosphate (NTP) or deoxynucleotide triphosphate (dNTP), and a single strand DNA binding protein (SSB). One or more additional reagents may also be included in the helicase preparation, where these are selected from the following: one or more additional helicases, an accessory protein, small molecules, chemical reagents and a buffer. Where a thermostable helicase is utilized in a helicase preparation, the presence of a single stranded binding protein is optional.

### Single-Stranded DNA Binding Proteins

Some helicases show improved activity in the presence of single-strand binding proteins (SSB). In these circumstances, the choice of SSB is generally not limited to a specific protein. Examples of single strand binding proteins are T4 gene 32 protein, E. coli SSB, T7 gp2.5 SSB, phage phi29 SSB (Kornberg and Baker, supra (1992)) and truncated forms of the aforementioned.

### Other Chemical Reagents

In addition to salt and pH, other chemical reagents, such as denaturation reagents including urea and dimethyl-sulfoxide (DMSO) can be added to the tHDA reaction to partially denature or de-stabilize the duplex DNA. These other chemical reagents can also be part of the helicase preparation. tHDA reactions can be compared in different concentrations of denaturation reagents with or without SSB protein. In this way, chemical compounds can be identified which increase tHDA efficiency and/or substitute for SSB in single-strand (ss) DNA stabilization. Most of the biomacromolecules such as nucleic acids and proteins are designed to function and/or form their native structures in a living cell at much high concentrations than in vitro experimental conditions. Polyethylene glycol (PEG) has been used to create an artificial molecular crowding condition by excluding water and creating electrostatic interaction with solute polycations (Miyoshi, et al., Biochemistry 41:15017 15024 (2002)). When PEG (7.5%) is added to a DNA ligation reaction, the reaction time is reduced to 5 min (Quick Ligation Kit, New England Biolabs, Inc. (Beverly, Mass.)). PEG has also been added into helicase unwinding assays to increase the efficiency of the reaction (Dong, et al., Proc. Natl. Acad. Sci. USA 93:14456 14461 (1996)). PEG or other molecular crowding reagents on HDA may increase the effective concentrations of enzymes and nucleic acids in tHDA reaction and thus reduce the reaction time and amount of protein concentration needed for the reaction.

### Cofactors

ATP or TTP is a common energy source for highly processive helicases. On average one ATP molecule is consumed by a DNA helicases to unwind 1 to 4 base pairs (Kornberg and Baker, supra (1992)). In some aspects of the described methods, a UvrD-based tHDA system had an optimal initial ATP concentration of 3 mM. To amplify a longer target, more ATP may be consumed as compared to a shorter target. In these circumstances, it may be desirable to include a pyruvate kinase-based ATP regenerating system for use with the helicase (Harmon and Kowalczykowski, Journal of Biological Chemistry 276:232 243 (2001)).

### Topoisomerase

Topoisomerase can be used in long tHDA reactions to increase the ability of tHDA to amplify long target amplicons. When a very long linear DNA duplex is separated by a helicase, the swivel (relaxing) function of a topoisomerase removes the twist and prevents over-winding (Kornberg and Baker, supra (1992)). For example, E. coli topoisomerase I (Fermentas, Vilnius, Lithuania) can be used to relax negatively supercoiled DNA by introducing a nick into one DNA strand. In contrast, E. coli DNA gyrase (topoisomerase II) introduces a transient double-stranded break into DNA allowing DNA strands to pass through one another (Kornberg and Baker, supra (1992)).

### Helicases

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W. H. Freeman and Company (2.sup.nd ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W.H. Freeman and Company (2nd ed. (1992)), include E. coli helicase I, II, III, & IV, Rep, DnaB, PriA, PcrA, T4 Gp4 lhelicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232 243 (2001)), thermostable UvrD helicases from T. tengcongensis (disclosed in this invention, Example XII) and T. thermophilus (Collins and McCarthy, Extremophiles. 7:35 41. (2003)), thermostable DnaB helicase from T. aquaticus (Kaplan and Steitz, J. Biol. Chem. 274:6889 6897 (1999)), and MCM helicase from archaeal and eukaryotic organisms ((Grainge et al., Nucleic Acids Res. 31:4888 4898 (2003)).

Examples of helicases for use in present embodiments may also be found at the Blocks WWW Server, a service for biological sequence analysis at the Fred Hutchinson Cancer Research Center. This site lists 49 Herpes helicases, 224 DnaB helicases, 250 UvrD-helicases and UvrD/Rep helicases, 276 DEAH_ATP-dependent helicases, 147 Papillom_El Papillomavirus helicase El protein, 608 Viral helicasel Viral (superfamily 1) RNA helicases and 556 DEAD ATP-dependent helicases. Examples of helicases that generally replicate in a 5′ to 3′ direction are T7 Gp4 helicase, DnaB helicase and Rho helicase, while examples of helicases that replicate in the 3′-5′ direction include UvrD helicase, PcrA, Rep, NS3 RNA helicase of HCV.

Helicases use the energy of nucleoside triphosphate (for example ATP) hydrolysis to break the hydrogen bonds that hold the strands together in duplex DNA and RNA (Kornberg and Baker, DNA Replication, W.H. Freeman and Company (2.sup.nd ed. (1992)), especially chapter 11). Helicases are involved in every aspect of nucleic acid metabolism in the cell such as DNA replication, DNA repair and recombination, transcription, and RNA processing. This widespread usage may be reflected by the large numbers of helicases found in all living organisms.

Helicases have been classified according to a number of different characteristics. For example, a feature of different helicases is their oligomeric structure including helicases with single or multimeric structures. For example, one family of helicases is characterized by hexameric structures while another family consists of monomeric or dimeric helicases.

Another characteristic of helicases is the occurrence of conserved motifs. All helicases have the classical Walker A and B motifs, associated with ATP-binding and Mg²⁺-binding (reviewed in Caruthers and McKay. Curr. Opin. Struct. Biol. 12:123 133 (2002), Soultanas and Wigley. Trends Biochem. Sci. 26:47 54 (2001)). Helicases have been classified into several superfamilies (Gorbalenya and Koonin. Curr. Opin. Struct. Biol. 3:419 429 (1993)) according to the number of helicase signature motifs and differences in the consensus sequences for motifs. Superfamilies 1 and 2 have seven characteristic helicase signature motifs and include helicases from archaea, eubacteria, eukaryotes and viruses, with helicases unwinding duplex DNA or RNA in either 3′ to 5′ direction or 5′ to 3′ direction. Examples of superfamily 1 helicases include the E. coli UvrD helicase, the T. tengcongensis UvrD helicase, and the B subunit of RecBCD. Superfamily 3 has three motifs and superfamily 4 has five motifs. Examples of superfamily 4 helicases include the T7 Gp4 helicase and DnaB helicases. A new family different from those canonical helicases is the AAA⁺ family (the extended family of ATPase associated with various cellular activities).

A third type of classification relates to the unwinding directionality of helicases i.e. whether the helicase unwinds the nucleic acid duplex in a 5′-3′ direction (such as T7 Gp4 helicase) or in a 3′-5′ direction (such UvrD helicase) based on the strand on which the helicase binds and travels.

A fourth type of classification relates to whether a helicase preferably unwinds blunt ended nucleic acid duplexes or duplexes with forks or single stranded tails. Blunt-ended nucleic acid duplexes may not be required in the first cycle of helicase-dependent amplification but are desirable in subsequent cycles of amplification because along with the progress of the amplification reaction the blunt-ended target fragment becomes the dominant species. These blunt-ended target nucleic acids form template substrates for subsequent rounds of amplification.

To accomplish the tHDA described herein, a helicase classified according to any of the above is suitable for nucleic acid amplification. according to the present methods to achieve helicase dependent amplification.

Regardless of the source of the target nucleic acid, a helicase preparation may be used to replace a heat denaturation step during amplification of a nucleic acid by unwinding a double stranded molecule to produce a single stranded molecule for polymerase dependent amplification without a change in temperature of reaction. Hence thermocycling that is required during standard PCR amplification using Taq polymerase can be avoided.

In general, the temperature of denaturation suitable for permitting specificity of primer-template recognition and subsequent annealing may occur over a range of temperatures, for example 20° C. to 75° C. For example, temperature may be selected according to which helicase is selected for the melting process. Tests to determine optimum temperatures for amplification of a nucleic acid in the presence of a selected helicase can be determined by routine experimentation by varying the temperature of the reaction mixture and comparing amplification products using gel electrophoresis.

Denaturation of nucleic acid hybrids or duplexes can be accelerated by using a thermostable helicase preparation under incubation conditions that include higher temperature for example in a range of 45° C. to 75. ° C. Performing HDA at high temperature using a thermostable helicase preparation and a thermostable polymerase may increase the specificity of primer binding, which can improve the specificity of amplification.

In certain aspects, it may be desirable to utilize a plurality of different helicase enzymes in an amplification reaction. The use of a plurality of helicases may enhance the yield and length of target amplification in HDA under certain conditions where different helicases coordinate various functions to increase the efficiency of the unwinding of duplex nucleic acids. For example, a helicase that has low processivity but is able to melt blunt-ended DNA may be combined with a second helicase that has great processivity but recognizes single-stranded tails at the border of duplex region for the initiation of unwinding. In this example, the first helicase initially separates the blunt ends of a long nucleic acid duplex generating 5′ and 3′ single-stranded tails and then dissociates from that substrate due to its limited processivity. This partially unwound substrate is subsequently recognized by the second helicase that then continues the unwinding process with superior processivity. In this way, a long target in a nucleic acid duplex may be unwound by the use of a helicase preparation containing a plurality of helicases and subsequently amplified in a HDA reaction.

5. Detection Labels

The methods described herein can also se used to detect a target nucleic acid sequence. Detection of the target nucleic acid can take place during or after the amplification reaction. To aid in detection and quantitation of target nucleic acids amplified using the disclosed compositions and methods, detection labels can be utilized. Detection labels can be directly incorporated into amplified target nucleic acids or can be coupled to amplified target nucleic acids. As used herein, a “detection label” is any molecule that can be associated with amplified target nucleic acid, directly or indirectly, and which results in a measurable, detectable signal, either directly or indirectly. Many such labels for incorporation into nucleic acids or coupling to nucleic acids are known to those of skill in the art. Examples of detection labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow SGF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Examples of fluorescent labels include fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037 and PCT Applications WO 97/17471 and WO 97/17076.

Labeled nucleotides are another form of detection label since they can be directly incorporated into the amplification products during synthesis. Examples of detection labels that can be incorporated into amplified target nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other preferred nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified target nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.13,7]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal. Labels can also be the disclosed reagent compositions.

Molecules that combine two or more of these detection labels are also considered detection labels. Any of the known detection labels can be used with the disclosed probes, tags, and method to label and detect target nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

### Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes, and QPNA probes. DxS′ Scorpion Primers as described in U.S. Pat. No. 7,445,900; Whitcombe, et al, 1999, Nature Biotech 17, 804-807; Thelwell, et al. (2000), Nucleic Acid Research 29, 3752-3761; Solinas, et al. (2001), Nucleic Acid Research 29, 1-9, all of which are hereby incorporated by reference for their teaching of Scorpion pimers, can also be used.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers. The use of several types of fluorescent change probes and primers are reviewed in Schweitzer and Kingsmore, Curr. Opin. Biotech. 12:21-27 (2001). Hall et al., Proc. Natl. Acad. Sci. USA 97:8272-8277 (2000), describe the use of fluorescent change probes with Invader assays.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes (Holland et al., Proc. Natl. Acad. Sci. USA 88:7276-7280 (1991)) are an example of cleavage activated probes.

### Modified TaqMan Probes

Also described herein are modified TaqMan probes. TaqMan probes are fluorescent change probes that involve a change in fluorescence intensity or wavelength based on a change in the form or conformation of the probe or primer and nucleic acid to be detected, assayed or replicated. For example, described herein are modified TaqMan probes that are comprised of a sequence that is complementary to a target sequence and additionally have a short tail at either the 3′ or 5′-end of the modified TaqMan probe complementary to the 5′ or 3′-end modified TaqMan probe, respectively. The short tail can assist in forming a stem-loop structure when the modified TaqMan probe is not hybridized to a target nucleic acid. The non-tail portion of the modified TaqMan probe is complementary to the target nucleic acid and is capable of hybridizing to a target nucleic acid. In some aspects, the short tail of the modified TaqMan probe can be complementary or non-complementary to the target.

The modified TaqMan probes can be used as a detection label in the methods described herein. The modified TaqMan probes are an improvement of molecular beacons and existing TaqMan probes as they are easier to open than a molecular beacon and the modified TaqMan probes quench more predictably and efficiently than existing TaqMan probes.

Cleavage quenched probes can also be used in the methods described herein. Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends a the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers (Nazerenko et al., Nucleic Acids Res. 25:2516-2521 (1997)) and scorpion primers (Thelwell et al., Nucleic Acids Res. 28(19):3752-3761 (2000)).

Cleavage activated primers are similar to cleavage activated probes except that they are primers that are incorporated into replicated strands and are then subsequently cleaved. Little et al., Clin. Chem. 45:777-784 (1999), describe the use of cleavage activated primers.

### Solid Supports

Solid supports are solid-state substrates or supports with which target nucleic acids or amplification products of the disclosed method (or other components used in, or produced by, the disclosed method) can be associated. Target nucleic acids can be associated with solid supports directly of indirectly. Amplification products can be associated with solid supports directly or indirectly. For example, amplification products can be bound to the surface of a solid support or associated with a capture antibody, or oligonucleotide probes immobilized on solid supports. An array detector is a solid support to which multiple different capture antibodies or oligonucleotide probes can be coupled in an array, grid, or other organized pattern. Target arrays are arrays of target nucleic acids attached to solid supports. Oligonucleoitude probe arrays are arrays of oligonucleotide probes attached to a solid support. Capture antibody arrays are arrays of capture antibodies attached to a solid support.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, polyamino acids or magnets. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. A useful form for a solid-state substrate is a microtiter dish. In some embodiments, a multiwell glass slide can be employed.

An array can include a plurality of components (such as target nucleic acids, target samples, detection labels, oligonucleotide probes, capture antibodies or amplification products) immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification of amplification products. Although useful, it is not required that the solid support be a single unit or structure. Sets of components can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including oligonucleotide probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Methods for immobilizing antibodies and other proteins to solid-state substrates are well established Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. Another example of an attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies and other proteins can be attached to a substrate by chemically cross-linking a free amino group on the antibody or protein to reactive side groups present within the solid-state substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

Each of the components immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### Solid-State Detectors

Solid-state detectors are solid supports to which oligonucleotide probes or capture antibodies have been coupled. A preferred form of solid-state detector is an array detector. An array detector is a solid-state detector to which multiple different oligonucleotide probes or capture antibodies have been coupled in an array, grid, or other organized pattern.

Solid-state substrates for use in solid-state detectors can include any solid material to which oligonucleotides can be coupled. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Capture antibodies immobilized on a solid-state substrate allow capture of double-stranded probe-target hybrids or their amplification targets on a solid-state detector. Such capture provides a convenient means of washing away reaction components that might interfere with subsequent method steps. By attaching different capture antibodies to different regions of a solid-state detector, different products can be captured at different, and therefore diagnostic, locations on the solid-state detector. For example, in a multiplex assay, oligonucleotide probes specific for numerous different target nucleic acids (each representing a different target nucleic acid sequence amplified via a different set of primers) can be immobilized in an array, each in a different location. Capture and detection will occur only at those array locations corresponding to amplified nucleic acids for which the corresponding target nucleic acid sequences were present in a sample.

### Oligonucleotide Synthesis

Oligonucleotide probes, oligonucleotide primers or any other oligonucleotides can be synthesized using established oligonucleotide synthesis methods. Methods to produce or synthesize oligonucleotides are well known. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. No. 6,294,664 and U.S. Pat. No. 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor, or a mixture of one or more different nucleotide precursors. In general, degenerate or random positions in an oligonucleotide can be produced by using a mixture of nucleotide precursors representing the range of nucleotides that can be present at that position. Thus, precursors for A and T can be included in the reaction for a particular position in an oligonucleotide if that position is to be degenerate for A and T. Precursors for all four nucleotides can be included for a fully degenerate or random position. Completely random oligonucleotides can be made by including all four nucleotide precursors in every round of synthesis. Degenerate oligonucleotides can also be made having different proportions of different nucleotides. Such oligonucleotides can be made, for example, by using different nucleotide precursors, in the desired proportions, in the reaction.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

So long as their relevant function is maintained, oligonucleotide primers, oligonucleotide probes, and any other oligonucleotides can be made up of or include modified nucleotides (nucleotide analogs). Many modified nucleotides are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

### Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for amplifying a target nucleic acid in a helicase dependent reaction, the kit comprising one or more reagent compositions and one or more components or reagents for capture of the target nucleic acid, tHDA amplification, detection of amplification products, or both. For example, the kits can include one or more reagent compositions and one or more oligonucleotide probes, one or more capture antibodies, one or more oligonucleotide primers, one or more detection labes, or a combination. Another form of kit can comprise a plurality of reagent compositions. The kits also can contain, for example, nucleotides, buffers, helicase, accessory proteins, topoisomerases, or a combination.

### Mixtures

Disclosed are mixtures formed by preparing the disclosed composition or performing or preparing to perform the disclosed methods. Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### Systems

Disclosed are systems useful for performing, or aiding in the performance of, the disclosed method. Also disclosed are systems for producing reagent compositions. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising solid supports and reagent compositions.

### Data Structures and Computer Control

Disclosed are data structures used in, generated by, or generated from, the disclosed method. Data structures generally are any form of data, information, and/or objects collected, organized, stored, and/or embodied in a composition or medium. A target fingerprint stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed compositions and methods are applicable to numerous areas including, but not limited to, detection and/or analysis of target nucleic acids, disease detection, protein detection, nucleic acid mapping, mutation detection, gene discovery, gene mapping, and agricultural research. Particularly useful are assays to amplify or detect target nucleic acids. Other uses include, for example, detection of target nucleic acids in samples, mutation detection; detection of sexually transmitted diseases such as Chlamydia trachomatis (CT) and Neisseria gonorrhoeae (NG).

### Methods

Disclosed herein are methods of amplifying a double stranded target nucleic acid in a helicase-dependent reaction. For example, disclosed herein, are methods of amplifying a double stranded target nucleic acid in a helicase-dependent reaction comprising: (a) denaturing the target nucleic acid; (b) contacting one or more oligonucleotide probes with the denatured target nucleic acid, wherein one or more of the oligonucleotide probes hybridize to the denatured target nucleic acid to form double-stranded probe-target hybrids; (c) contacting the double-stranded probe-target hybrids with one or more capture antibodies wherein the one or more capture antibodies hybridize to the double-stranded probe-target hybrids to form captured double-stranded probe-target hybrids, (d) removing all uncaptured nucleic acids; (e) adding one or more oligonuceotide primers, wherein the oligonucleotide primers hybridize to the target nucleic acid; (f) synthesizing an extension product of the oligonucleotide primers which is complementary to the target nucleic acid, by means of a DNA polymerase to form a target nucleic acid duplex; (g) contacting the target nucleic acid duplex of step (f) with a helicase preparation and amplifying the target nucleic acid duplex in a helicase-dependent reaction. This method can be carried out in separate steps, for example, step (a) can be carried out first and then step (b), then step (c), etc. In addition, this method can be carried out wherein steps (e), (f) and (g) or steps (f) and (g) are carried out simultaneously.

The double stranded target nucleic acid can be isolated from a sample prior to step (a) or the double stranded target nucleic acid can be in a target nucleic acid sample. In other words, the methods can be carried out directly on a sample. The sample can be any of the samples described herein, including, but not limited to blood, urine, stool, saliva, tear, bile cervical, urogenital, nasal swabs, sputum, or other biological sample.

In the event that the double-stranded target nucleic acid is DNA the polynucleotide probes can be RNA. Alternatively, in the event that the double-stranded target nucleic acid is RNA the polynucleotide probes can be DNA.

Amplification can also be conducted under isothermal conditions as described elsewhere herein. A “helicase dependent reaction” is an amplification reaction that does not occur in the absence of the helicase as determined by gel electrophoresis. In the methods described herein, helicase preparations are used. In some aspects, the helicase preparation comprises a helicase and optionally a single strand binding protein. In some aspects, the helicase preparation comprises a helicase and a single strand binding protein (SSB) unless the helicase preparation comprises a thermostable helicase wherein the single strand binding protein is optional.

Also disclosed herein, are methods of amplifying a double stranded target nucleic acid in a helicase-dependent reaction comprising: (a) denaturing the target nucleic acid; (b) contacting one or more oligonucleotide probes with the denatured target nucleic acid, wherein one or more of the oligonucleotide probes hybridize to the denatured target nucleic acid to form double-stranded probe-target hybrids; (c) contacting the double-stranded probe-target hybrids with one or more capture antibodies, wherein the hybrid capture antibodies comprise a magnetic bead and wherein the one or more capture antibodies hybridize to the double-stranded probe-target hybrids to form captured double-stranded probe-target hybrids, (d) removing all uncaptured nucleic acids; (e) adding one or more oligonuceotide primers, wherein the oligonucleotide primers hybridize to the target nucleic acid; (f) synthesizing an extension product of the oligonucleotide primers which is complementary to the target nucleic acid, by means of a DNA polymerase to form a target nucleic acid duplex; (g) contacting the target nucleic acid duplex of step (f) with a helicase preparation and amplifying the target nucleic acid duplex in a helicase-dependent reaction.

In the methods described herein, the one or more oligonucleotide primers added in step (e) can be used for synthesizing an extension product of the oligonucleotide primers which is complementary to the target nucleic acid as well as for the helicase-dependent reaction. For example, the primer extended in step (e) can also serve as a forward or reverse primer in the helicase-dependent reaction. Alternatively, different oligonucleotide primers can be added in step (e) and in the helicase preparation. In some aspects, the oligonucleotide primers and probes can be designed to minimize the possibility of hybridizing to one another. Methods of oligonucleotide primer and probe design are described elsewhere herein. In addition, the oligonucleotide primers and probes can be designed to minimize overlap with their congnate target. Although some overlap will not prohibit the reactions from taking place, overlap should be minimized between the olionucleotide primers and probes.

Also disclosed herein, are methods of amplifying a double stranded target nucleic acid in a helicase-dependent reaction comprising: (a) denaturing the target nucleic acid; (b) contacting one or more oligonucleotide probes with the denatured target nucleic acid, wherein one or more of the oligonucleotide probes hybridize to the denatured target nucleic acid to form double-stranded probe-target hybrids; (c) contacting the double-stranded probe-target hybrids with one or more capture antibodies, wherein the one or more capture antibodies hybridize to the double-stranded probe-target hybrids to form captured double-stranded probe-target hybrids, (d) removing all uncaptured nucleic acids; (e) adding one or more oligonuceotide primers, wherein the oligonucleotide primers hybridize to the target nucleic acid; (f) synthesizing an extension product of the oligonucleotide primers which is complementary to the target nucleic acid, by means of a DNA polymerase to form a target nucleic acid duplex; (g) contacting the target nucleic acid duplex of step (f) with a helicase preparation and amplifying the target nucleic acid duplex in a helicase-dependent reaction, wherein one or more of the oligonucleotide primers are present in different concentrations. For example, disclosed are methods wherein the primers designed to hybridize to the same strand of the target nucleic acid as the olionucleotide probes are present at alower concentration that the oligonucleotides designed to hybridize to the complement of the strand of the target nucleic acid that the olionucleotide probes are designed to hybridized to. Such, oligonucleotide concentration asymmetry allows for the oligonucleotide probes to hybridize to the target nucleic acid sequence easier, with less competition.

In some aspects, denaturing the target nucleic acid can comprise heating the target nucleic acid to denature the target nucleic acid. In some aspects, denaturing the target nucleic acid can comprise incubating the target nucleic acid in the presence of NaOH prior to contacting one or more oligonucleotide probes with the denatured target nucleic acid. On other aspects, denaturing the target nucleic acid can comprise incubating the target nucleic acid at 65° C. for 10 minutes in the presence of 50 mM NaOH prior to contacting one or more oligonucleotide probes with the denatured target nucleic acid.

Also disclosed herein, are methods of amplifying a double stranded target nucleic acid in a helicase-dependent reaction comprising: (a) denaturing the target nucleic acid; (b) contacting one or more oligonucleotide probes with the denatured target nucleic acid, wherein one or more of the oligonucleotide probes hybridize to the denatured target nucleic acid to form double-stranded probe-target hybrids; (c) contacting the double-stranded probe-target hybrids with one or more capture antibodies, wherein the one or more capture antibodies hybridize to the double-stranded probe-target hybrids to form captured double-stranded probe-target hybrids, (d) removing all uncaptured nucleic acids; (e) adding one or more oligonuceotide primers, wherein the oligonucleotide primers hybridize to the target nucleic acid; (f) synthesizing an extension product of the oligonucleotide primers which is complementary to the target nucleic acid, by means of a DNA polymerase to form a target nucleic acid duplex; (g) contacting the target nucleic acid duplex of step (f) with a helicase preparation and amplifying the target nucleic acid duplex in a helicase-dependent reaction, wherein the method further comprises detecting the target nucleic acid. Detection can be carried out by adding a detection label to the reaction mixture. For example, disclosed herein are methods, wherein a detection label is added during or after steps (a) through (g). Also disclosed are methods, wherein a detection label is added during or after step (e), (f) or (g). Detection can take place during, after or during and after the amplification reaction (for example the helicase dependent reaction). The target nucleic acid can be detected by end point fluorescent detection.

Also disclosed are methods of amplifying a single stranded target nucleic acid in a helicase-dependent reaction, comprising: (a) contacting one or more oligonucleotide probes with the single stranded target nucleic acid, wherein one or more of the oligonucleotide probes hybridize to the target nucleic acid to form double-stranded probe-target hybrids; (b) contacting the double-stranded probe-target hybrids with one or more capture antibodies, wherein the one or more of capture antibodies hybridize to the double-stranded probe-target hybrids to form captured double-stranded probe-target hybrids, (c) removing all uncaptured nucleic acids; (d) adding one or more oligonuceotide primers, wherein the oligonucleotide primers hybridize to the target nucleic acid; (e) synthesizing an extension product of the oligonucleotide primers which is complementary to the target nucleic acid, by means of a DNA polymerase to form a target nucleic acid duplex; (f) contacting the target nucleic acid duplex of step (e) with a helicase preparation and amplifying the target nucleic acid duplex in a helicase-dependent reaction.

The single stranded target nucleic acid can be any single stranded nucleic acid, including RNA, DNA, cDNA or any other nucleic acid as described elsewhere herein.

In the event that the single stranded target nucleic acid is RNA, DNA oligonucleotide probes can be used. In some aspects where single stranded target nucleic acid is mRNA, reverse transcription can be carried out prior to step (a) wherein the mRNA is reverse transcribed to form cDNA. In the event that mRNA is reverse transcribed to form cDNA, RNA oligonucleotide probes can be used. In some aspects where mRNA is reverse transcribed to form cDNA prior to step (a), after the reverse transcription reaction the mRNA can be degraded prior to or during step (a) or no mRNA deredation can take place. In some aspects where single stranded target nucleic acid is mRNA, the mRNA itself can act as the single stranded target nucleic acid. In such aspects, step (e) can further comprise a reverse transcription reaction, whereby the oligonucleotide primers of step (e) can serve to prime a reverse transcription reaction to form cDNA. The cDNA can then serve as a template for primer extension to form a cDNA target nucleic acid duplex.

In some aspects, the methods of amplifying a single stranded target nucleic acid sequence, the methods can be carried out in separate steps, for example, step (a) can be carried out first and then step (b), then step (c), etc. In addition, this method can be carried out wherein steps (e), (f) and (g) or steps (f) and (g) are carried out simultaneously.

The single stranded target nucleic acid can be isolated from a sample prior to step (a) or the double stranded target nucleic acid can be in a target nucleic acid sample. In other words, the methods can be carried out directly on a sample. The sample can be any of the samples described herein, including, but not limited to blood, urine, stool, saliva, tear, bile cervical, urogenital, nasal swabs, sputum, or other biological sample.

In the event that the single-stranded target nucleic acid is DNA the polynucleotide probes can be RNA. Alternatively, in the event that the single-stranded target nucleic acid is RNA the polynucleotide probes can be DNA.

Amplification can also be conducted under isothermal conditions as described elsewhere herein. A “helicase dependent reaction” is an amplification reaction that does not occur in the absence of the helicase as determined by gel electrophoresis. In the methods described herein, helicase preparations are used. In some aspects, the helicase preparation comprises a helicase and optionally a single strand binding protein. In some aspects, the helicase preparation comprises a helicase and a single strand binding protein (SSB) unless the helicase preparation comprises a thermostable helicase wherein the single strand binding protein is optional.

Also disclosed are methods of amplifying a single stranded target nucleic acid in a helicase-dependent reaction, comprising: (a) contacting one or more oligonucleotide probes with the single stranded target nucleic acid, wherein one or more of the oligonucleotide probes hybridize to the target nucleic acid to form double-stranded probe-target hybrids; (b) contacting the double-stranded probe-target hybrids with one or more capture antibodies, wherein the hybrid capture antibodies comprise a magnetic bead and wherein the one or more of capture antibodies hybridize to the double-stranded probe-target hybrids to form captured double-stranded probe-target hybrids, (c) removing all uncaptured nucleic acids; (d) adding one or more oligonuceotide primers, wherein the oligonucleotide primers hybridize to the target nucleic acid; (e) synthesizing an extension product of the oligonucleotide primers which is complementary to the target nucleic acid, by means of a DNA polymerase to form a target nucleic acid duplex; (f) contacting the target nucleic acid duplex of step (e) with a helicase preparation and amplifying the target nucleic acid duplex in a helicase-dependent reaction.

In the methods described herein, the one or more oligonucleotide primers added in step (e) can be used for synthesizing an extension product of the oligonucleotide primers which is complementary to the target nucleic acid as well as for the helicase-dependent reaction. For example, the primer extended in step (e) can also serve as a forward or reverse primer in the helicase-dependent reaction. Alternatively, different oligonucleotide primers can be added in step (e) and in the helicase preparation. In some aspects, the oligonucleotide primers and probes can be designed to minimize the possibility of hybridizing to one another. Methods of oligonucleotide primer and probe design are described elsewhere herein. In addition, the oligonucleotide primers and probes can be designed to minimize overlap with their congnate target. Although some overlap will not prohibit the reactions from taking place, overlap should be minimized between the olionucleotide primers and probes.

Also disclosed are methods of amplifying a single stranded target nucleic acid in a helicase-dependent reaction, comprising: (a) contacting one or more oligonucleotide probes with the single stranded target nucleic acid, wherein one or more of the oligonucleotide probes hybridize to the target nucleic acid to form double-stranded probe-target hybrids; (b) contacting the double-stranded probe-target hybrids with one or more capture antibodies, wherein the one or more of capture antibodies hybridize to the double-stranded probe-target hybrids to form captured double-stranded probe-target hybrids, (c) removing all uncaptured nucleic acids; (d) adding one or more oligonuceotide primers, wherein the oligonucleotide primers hybridize to the target nucleic acid; (e) synthesizing an extension product of the oligonucleotide primers which is complementary to the target nucleic acid, by means of a DNA polymerase to form a target nucleic acid duplex; (f) contacting the target nucleic acid duplex of step (e) with a helicase preparation and amplifying the target nucleic acid duplex in a helicase-dependent reaction, wherein one or more of the oligonucleotide primers are present in different concentrations. For example, disclosed are methods wherein the primers designed to hybridize to the same strand of the target nucleic acid as the olionucleotide probes are present at alower concentration that the oligonucleotides designed to hybridize to the complement of the strand of the target nucleic acid that the olionucleotide probes are designed to hybridized to. Such, oligonucleotide concentration asymmetry allows for the oligonucleotide probes to hybridize to the target nucleic acid sequence easier, with less competition.

Amplified nucleic acid product may be detected by various methods including ethidium-bromide staining and detecting the amplified sequence by means of a label selected from the group consisting of a radiolabel, a fluorescent-label, and an enzyme. For example HDA amplified products can be detected in real-time using fluorescent-labeled LUXTM Primers (Invitrogen Corporation, Carlsbad, Calif.) which are oligonucleotides designed with a fluorophore close to the 3′ end in a hairpin structure. This configuration intrinsically renders fluorescence quenching capability without separate quenching moiety. When the primer becomes incorporated into double-stranded amplification product, the fluorophore is dequenched, resulting in a significant increase in fluorescent signal.

For example, disclosed are methods of amplifying a single stranded target nucleic acid in a helicase-dependent reaction, comprising: (a) contacting one or more oligonucleotide probes with the single stranded target nucleic acid, wherein one or more of the oligonucleotide probes hybridize to the target nucleic acid to form double-stranded probe-target hybrids; (b) contacting the double-stranded probe-target hybrids with one or more capture antibodies, wherein the one or more of capture antibodies hybridize to the double-stranded probe-target hybrids to form captured double-stranded probe-target hybrids, (c) removing all uncaptured nucleic acids; (d) adding one or more oligonuceotide primers, wherein the oligonucleotide primers hybridize to the target nucleic acid; (e) synthesizing an extension product of the oligonucleotide primers which is complementary to the target nucleic acid, by means of a DNA polymerase to form a target nucleic acid duplex; (f) contacting the target nucleic acid duplex of step (e) with a helicase preparation and amplifying the target nucleic acid duplex in a helicase-dependent reaction, wherein the method further comprises detecting the target nucleic acid. Detection can be carried out by adding a detection label to the reaction mixture. For example, disclosed herein are methods, wherein a detection label is added during or after steps (a) through (g). Also disclosed are methods, wherein a detection label is added during or after step (e), (f) or (g). Detection can take place during, after or during and after the amplification reaction (for example the helicase dependent reaction). The target nucleic acid can be detected by end point fluorescent detection.

In some aspects, parts of the disclosed methods can be carried out in a homogenous assay. A “homogenous assay” is an assay wherein amplification and detection of a target nucleic acid takes place in the same reaction. A homogenous assay can be an assay that generates a detectable signal during or after the amplification of a target nucleic acid. For example, steps (e) through (g) can be conducted in a homogenous assay.

In some aspects of the methods described herein, sugars and/or other additives can be used to stabilize the polymerases or helicases used at high temperature. Additives can be added independently of the other reagents or they can be a part of the helicase preparation. For example, additives for use in the disclosed amplification method are any compound, composition, or combination that can allow a thermolabile nucleic acid polymerase to perform template-dependent polymerization at an elevated temperature. Additives generally have a thermostabilizing effect on the nucleic acid polymerase. Additives allow a thermolabile nucleic acid polymerase to be used at temperature above the normal active range of the polymerase. Useful additives include sugars, chaperones, proteins, saccharides, amino acids, polyalcohols and their derivatives, and other osmolytes. Useful sugars include trehalose, glucose and sucrose. Useful saccharides include oligosaccharides and monosaccharides such as trehalose, maltose, glucose, sucrose, lactose, xylobiose, agarobiose, cellobiose, levanbiose, quitobiose, 2-β-glucuronosylglucuronic acid, allose, altrose, galactose, gulose, idose, mannose, talose, sorbitol, levulose, xylitol, arabitol, and polyalcohols such as glycerol, ethylene glycol, polyethylene glycol. Useful amino acids and derivatives thereof include N^{e}-acetyl-β-lysine, alanine, γ-aminobutyric acid, betaine, N^{α}-carbamoyl-L-glutamine 1-amide, choline, dimethylthetine, ecotine (1,4,5,6-tetrahydro-2-methyl-4-pirymidine carboxilic acid), glutamate, β-glutammine, glycine, octopine, proline, sarcosine, taurine and trymethylamine N-oxide (TMAO). Useful chaperone proteins include chaperone proteins of Thermophilic bacteria and heat shock proteins such as HSP 90, HSP 70 and HSP 60. Other useful additives include sorbitol, mannosylglycerate, diglycerol phosphate, and cyclic-2,3-diphosphoglycerate. Combinations of compounds and compositions can be used as additives.

In some aspects, the additive can be selected from the group consisting of sugars, chaperones, proteins, saccharides, amino acids, polyalcohols, and their derivatives, other osmolytes, amino acid derivatives, and chaperone proteins. For example, the additive can be selected from the group consisting of DMSO, betaine, sorbitol, dextran sulfate and mixtures thereof. In some aspects where DMSO is used as an additive, DMSO can be used at a final concentration of between 1 and 2%. In some aspects where betaine is used as an additive, betaine can be used at a final concentration of 0.1M-0.5M. In some aspects where sorbitol is used as an additive, sorbitol can be used at a final concentration of 0.1M-0.3M. In some aspects where dextran sulfate is used as an additive, dextran sulfate can be used at a final concentration of 10 pM-1 nM.

Also disclosed herein are methods of amplifying more than one target nucleic acid in a single reaction. The methods described herein can be multiplexed by using sets of different reagent compositions (having different oligonucleotide probes and different oligonucleotide primers), each reagent composition being associated with, for example, different target nucleic acids and/or array positions. For example, disclosed herein are methods of amplifying Chlamydia trachomatis (CT) and Neisseria gonorrhoeae (NG) in the same reaction (See for example, Example 5), wherein RNA oliognuceotide probes specific to either the multi-copy Opa gene (for NG), the cryptic plasmid (for CT) or the outer membrane protein (OMP) gene (for CT) were used in combination with oligonuceotide primers specific to the same.

Also disclosed herein are methods of amplifying two double stranded target nucleic acids in a single helicase-dependent reaction, wherein the two double stranded target nucleic acids comprise a first and a second double stranded target nucleic acids comprising: (a) denaturing the target nucleic acids; (b) contacting the first denatured target nucleic acid with one or more oligonucleotide probes wherein the oligonucleotide probes hybridize to the first denatured target nucleic acid to form first target double-stranded probe-target hybrids, and contacting the second denatured target nucleic acid with one or more oligonucleotide probes wherein the oligonucleotide probes hybridize to the second denatured target nucleic acid to form second target double-stranded probe-target hybrids; (c) contacting the first and second double-stranded probe-target hybrids with one or more capture antibodies, wherein the one or more capture antibodies bind to the first and second double-stranded probe-target hybrids to form captured first and second double-stranded probe-target hybrids, (d) removing all uncaptured nucleic acids; (e) adding one or more first target oligonuceotide primers, wherein the first target oligonucleotide primers hybridize to the first target nucleic acid and adding one or more second target oligonuceotide primers, wherein the second target oligonucleotide primers hybridize to the second target nucleic acid; (f) synthesizing extension products of the first and second target oligonucleotide primers which are complementary to the first and second target nucleic acids, respectively, by means of a DNA polymerase to form first and second target nucleic acid duplexes; (g) contacting the first and second target nucleic acid duplexes of step (f) with a helicase preparation and amplifying the target nucleic acid duplexes in a helicase-dependent reaction, wherein the helicase preparation comprises one or more primers that hybridize to the first target nucleic acid and further comprises one or more primers that hybridize to the second target nucleic acid.

Also disclosed herein are methods of amplifying two single stranded target nucleic acids in a single helicase-dependent reaction, wherein the two single stranded target nucleic acids comprise a first and a second single stranded target nucleic acids comprising: (a) contacting the first denatured target nucleic acid with one or more oligonucleotide probes wherein the oligonucleotide probes hybridize to the first denatured target nucleic acid to form first target double-stranded probe-target hybrids, and contacting the second denatured target nucleic acid with one or more oligonucleotide probes wherein the oligonucleotide probes hybridize to the second denatured target nucleic acid to form second target double-stranded probe-target hybrids; (b) contacting the first and second double-stranded probe-target hybrids with one or more capture antibodies, wherein the one or more capture antibodies bind to the first and second double-stranded probe-target hybrids to form captured first and second double-stranded probe-target hybrids, (c) removing all uncaptured nucleic acids; (d) adding one or more first target oligonuceotide primers, wherein the first target oligonucleotide primers hybridize to the first target nucleic acid and adding one or more second target oligonuceotide primers, wherein the second target oligonucleotide primers hybridize to the second target nucleic acid; (e) synthesizing extension products of the first and second target oligonucleotide primers which are complementary to the first and second target nucleic acids, respectively, by means of a DNA polymerase to form first and second target nucleic acid duplexes; (f) contacting the first and second target nucleic acid duplexes of step (e) with a helicase preparation and amplifying the target nucleic acid duplexes in a helicase-dependent reaction, wherein the helicase preparation comprises one or more primers that hybridize to the first target nucleic acid and further comprises one or more primers that hybridize to the second target nucleic acid.

When amplifying or detecting one or more target nucleic acids in a single reaction, the design of oligonucleotide probes and primers becomes important. Each oligonucleotide primer or probe should be designed to be specific to its cognate target nucleic acid sequence. Care should also be taken to avoid primer dimers or primer probe dimers to make the method more efficient. In addition, capture antibodies can differ or one can use the same capture antibodies to capture different double-stranded probe-target hybrids.

Use of different detection labels to identify different target nucleic acids can also be used. For example, associating different detection labels with different target nucleic acids, each different target nucleic acid can be detected by differential detection of the various detection labels. This can be accomplished, for example, by designing a different TaqMan probe for each target nucleic acid. Amplification of the different target nucleic acids can be detected based on different omplement portion sequences of the target nucleic acids by using, for example, oligonucloetide primers that are fluorescent change primers.

### EXAMPLES

### Example 1

### Alkali Target Denaturation

As helicase is able to unwind duplex DNA enzymatically, whether the entire tHDA reaction can be performed at one temperature at 65° C. without prior heat denaturation at 95° C. was tested. In addition, whether heat denaturation could be substituted by chemical alkali denaturation at 65° C. was also tested. Neisseria gonorrhoeae (NG) and Chlamydia trachomatis (CT) genes were chosen for the multiplex tHDA reaction as targets. Sodium hydroxide was added (example 1a.) to the CT and NG targets or to NG targets (example 1b.) and incubated at 65° C. for 10 min. For the control reaction, targets were diluted in H₂O. Following target denaturation, the tHDA reaction was performed and specific targets were detected using either the Luminex assay (example 1a.) or the real-time and endpoint fluorescence detection (examples 1b. and 1c.).

### Example 1a

### Evaluation of Alkaline Target Denaturation in CT/NG Multiplex Assay

The nucleic acid targets for this example were CT Cryptic Plasmid and NG Genomic DNA. To amplify CT in the tHDA reaction, ORF 3F and ORF 3R oligonucleotide primers were used (5′-ATCGCATGCAAGATATCGAGTATGCGT-3′ (SEQ ID NO. 185) and 5′Bio-CTCATAATTAGCAAGCTGCCTCAGAAT-3′ (SEQ ID NO. 186), respectively). To amplify NG in the tHDA reaction, opaD F and opaD R oligonucleotide primers were used (5′-TTGAAACACCGCCCGGAA-3′ (SEQ ID NO. 221) and 5′-TTTCGGCTCCTTATTCGGTTTAA-3′(SEQ ID NO. 222), respectively). The primer concentrations for the opaD F and opaD R were 30 nM and 75 nM, respectively.

The helicase preparation for the tHDA reaction also included Mg504: 3.5 mM; NaCl: 40 mM; dNTP: 0.4 mM; dATP: 3 mM; Bst Polymerase; 0.4 U/ul; Helicase: 3 ng/ul; and Betaine: 1M. The reaction was carried out for 10 minutes incubation/denaturation in NaOH at 65° C.; 90 minutes amplification at 65° C.

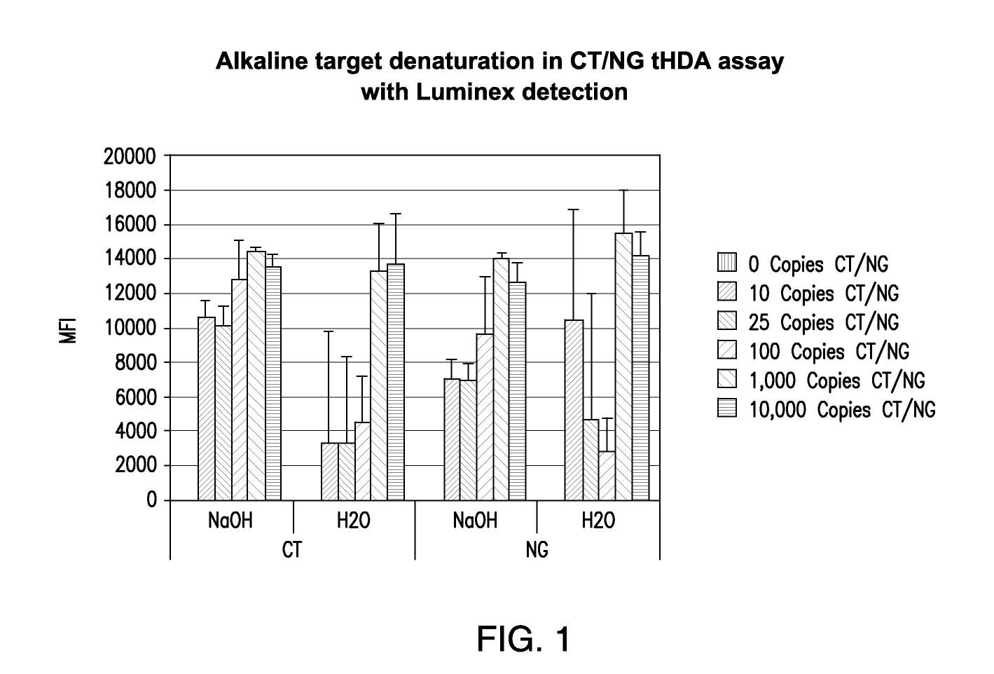

The results from this experiments showed that target denaturation in NaOH can result in an improved signal in multiplex CT/NG tHDA assay with Luminex detection, especially for low copy numbers of a CT target. Comparable sensitivity can also be achieved in a Luminex-based assay without alkaline denaturation. However, tHDA with NaOH denaturation can produce more consistent results with decreased variability (% CV). More variability (higher % CV) was seen with lower copy targets (10 and 25 copies) for non-alkaline target denaturation. (See FIG. 1).

### Example 1b

### Comparison of Target Denaturation Method in a tHDA Opa/Por Multiplex Assay

In this experiment, two different target nucleic acid sequences were used to identify the presence of CT and NG (opa and por, respectively). To begin, Neisseria gonorrhea genomic DNA in concentrations of 0, 10, 102 and 105 copies/assay were individually diluted either in 0.1M NaOH or water and then denatured at 65° C. for 10 min. The Neisseria gonorrhoeae genomic DNA was then subjected to real-time tHDA. For the tHDA reaction, the helicase preparation comprised 3.5 mM Mg2⁺, 40 mM NaCl, 0.4 mM dNTP, 3 mM dATP, 5 U rBST, 0.5 U Helicase, 0.2M Betaine, and 1% DMSO. In addition, TaqMan Probes: OpaD b1_Tex; CGTCCTTCAACATCAGTGAAAATCG (SEQ ID NO. 132) conjugated to Tex615 and porA5_VD5_Cy5; CGCCTATACGCCTGCTACTTTCACG (SEQ ID NO. 133) conjugated to Cy5 (80 nM each) were also added to the helicase preparation.

opaDv F1_{—}6/R1 (SEQ ID NOS. 228 and 229, respectively) and porA F5/R5 (SEQ ID NOS. 230 and 231, respectively) (40/120 nM) primers were used to amplify opaD (NG) and porA (CT), respectively.

Once the helicase preparation is added to the denatured Neisseria gonorrhoeae genomic DNA, the reaction mixture was incubated on a real-time thermocycler instrument for 6 min. 65° C. initial step, followed by 120 cycles (60 sec. each) at 65° C. After the amplification at 65° C. the cycler will automatically cycle 25° C. endpoint detection.When amplification was completed, the reaction mixture can be removed from the thermocycler and placed in −20° C. freezer.

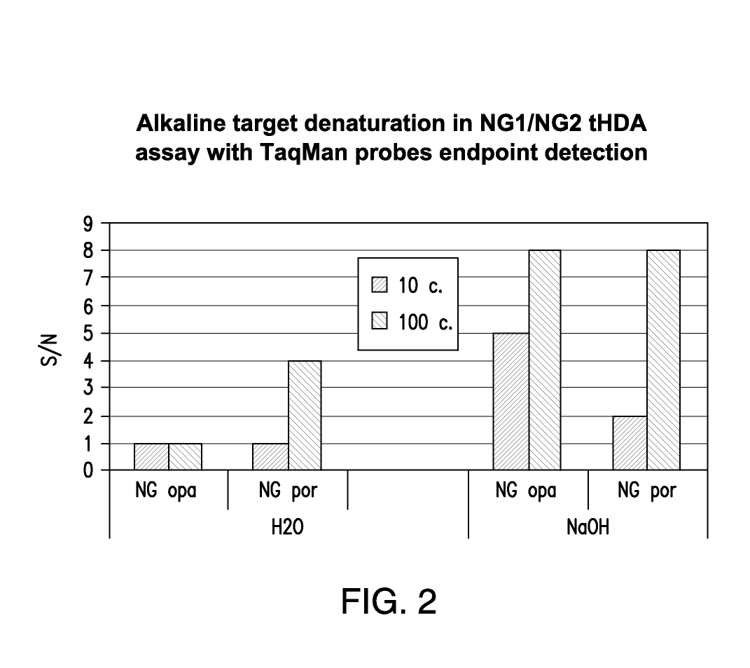

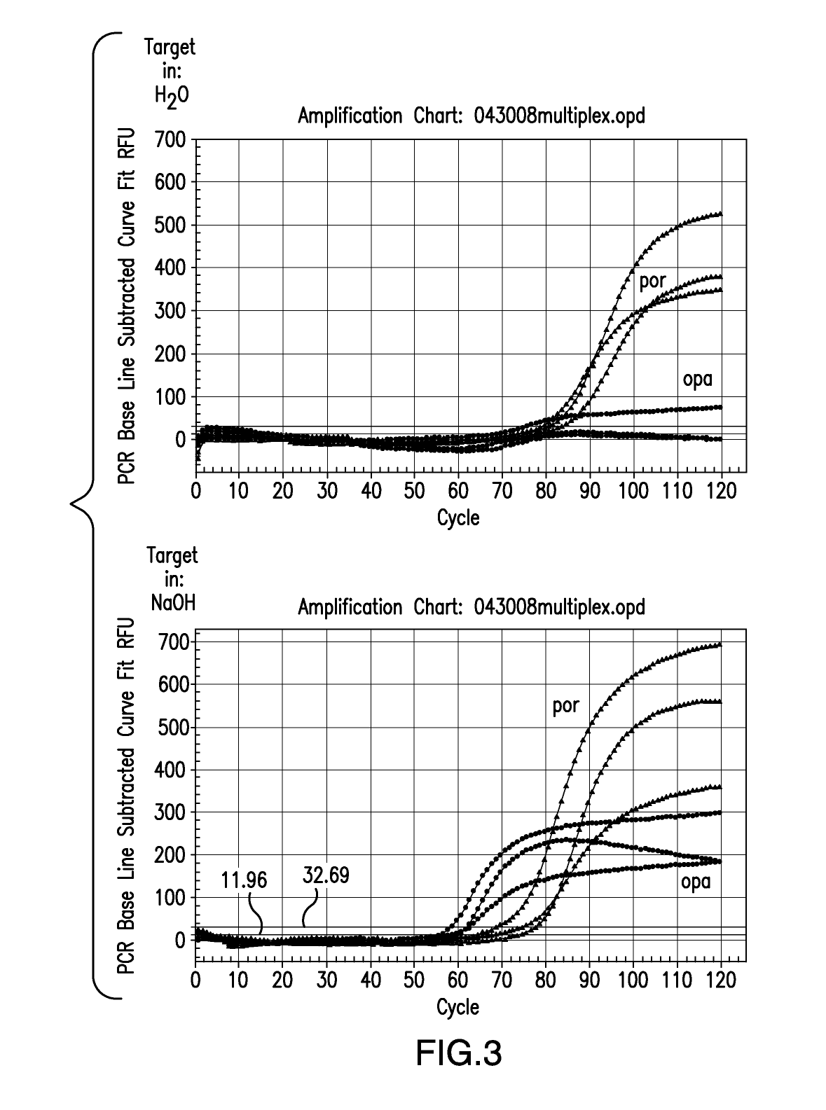

The results showed that target denaturation in NaOH improved signal to noise ratio in multiplex CT/NG tHDA with endpoint fluorescence detection. (See FIG. 2). The results showed that target denaturation in NaOH facilitates earlier amplification (lower Ct values). (See FIG. 3).

### Example 2

### Hybrid Capture Sample Prep Combined with tHDA

Hybrid Capture sample preparation was evaluated as a possible pre-analytical platform for a CT/NG multiplex tHDA assay. Front end hybrid capture (FE-HC) utilizing synRNA has been previously evaluated for both CT and NG targets. 20 contiguous RNA oligonucloetide probes (also referred to as syn RNA) over a span of 1 KB (50 nt each) were designed around capture probe and primer regions for the CT target nucleic acid. 22 contiguous RNA oligonucloetide probes (also referred to as syn RNA) (50 nt each) were designed for the NG target nucleic acid around the capture probe and primer regions. For the experiments described herein, RNA oligonucloetide probes for NG gene opaD were initially designed as 20 strands of 50mer RNA oligonucloetide probes. Additionally, the NG-specific RNA oligonucloetide probes were adjusted to smaller oligo strands, forming 22 oligos of 30 nt each. This set was designed without amplicon overlap, which consistently worked the best with both real-time and endpoint detection of NG opaD targets.

Examples 2c demonstrate the use of opaD-specific RNA oligonucloetide probes in the Hybrid Capture assay followed by real-time tHDA with EvaGreen or endpoint fluorescence detection.

### Example 2a

### Detection of CT Palsmid by tHDA with Hybrid Capture Sample Preparation and Luminex Assay

The target nucleic acid for this example was CT-1B Cryptic Plasmid. 20 contiguous 50mer RNA oligonucloetide probes specific to the CT plasmid were designed around the ORF capture probe and primer regions. The capture probe for this reaction was the Luminex

Capture Probe: CT—ORF LMX CP (5′-/5AmMC12/GGTAAAGCTCTGATATTTGAAGACTCTACTGAG-3′) (SEQ. ID. NO. 232). One or more of the following RNA oligonucloetide probes specific to the CT plasmid provided in Table 1 were also used:

Each of the above listed RNA oligonucloetide probes specific to the CT plasmid start at nucleotide 1786 of the CT plasmid (GenBank accession number: X06707). The 50mer RNA oligonucloetide probes of Table 1 were designed to hybridize to the same strand as the ORF 3F primer. Protein G beads: 2.5E+6 beads/assay” were used in this reaction.

tHDA was carries out using a helicase preparation comprising 15 nM of CT ORF Forward primer (5′-ATCGCATGCAAGATATCGAGTATGCGT-3′, SEQ ID NO. 189) and 75 nM of CT ORF Reverse primer (5′-CTCATAATTAGCAAGCTGCCTCAGAAT-3′, SEQ ID NO. 190); 4 mM Mg504; 40 mM NaCl; 0.4 mM dNTP; 3 mM dATP; 20 U Bst DNA Polymerase; and 1 U Tte-UvrD Helicase in a 50 ul reaction volume. The tHDA reaction was then carried out at 65° C. for 90 minutes.

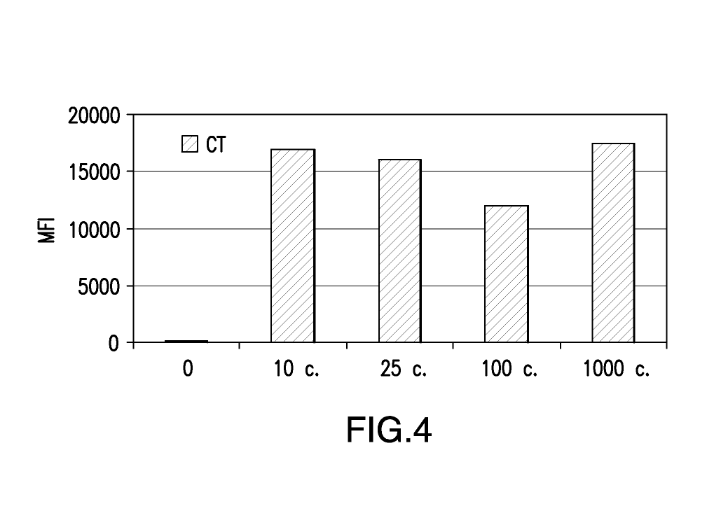

The results of this evaluation indicated that FE-HC is compatible with tHDA amplification. (See FIG. 4). RNA oligonucleotide probes can be used in FE-HC before tHDA with CT plasmid. HC sample preparation combined with tHDA can therefore eliminate the need for target denaturation.

### Example 2b

### Detection of Chlamydia and Gonorrhea Cells by Multiplex tHDA with Hybrid Capture Sample Preparation and Luminex Assay

The sample comprising the target nucleic acids for this example were CT Elementary Bodies and NG Viable Cells. 20 contiguous 50mer RNA oligonucloetide probes specific to CT and 34 contiguous 30mer RNA oligonucleotide probes specific to NG were designed around the ORF capture probe and primer regions. The RNA oligonucleotide probes for CT are described above in Table 1. One or more of the following RNA oligonucloetide probes specific to the NG provided in Table 3 were also used.

The 30mer RNA oligonucloetide probes of Table 9 were designed to hybridize to the same strand as the ORF 3F. Additionally, it was determined that the oligonucleotide probes can be between 15 and 100 nucleotides. For example, the oligonucleotide probes can be between 20 and 30 nucleotides long.

tHDA was carried out using a helicase preparation comprising 15 nM of CT ORF Forward primer (5′-ATCGCATGCAAGATATCGAGTATGCGT-3′, SEQ ID NO. 189) and 75 nM of CT ORF Reverse primer (5′-CTCATAATTAGCAAGCTGCCTCAGAAT-3′, SEQ ID NO. 190); 4 mM Mg504; 40 mM NaCl; 0.4 mM dNTP; 3 mM dATP; 20 U Bst DNA Polymerase; and 1 U Tte-UvrD Helicase in a 50 ul reaction volume. The tHDA reaction was then carried out at 65° C. for 90 minutes.

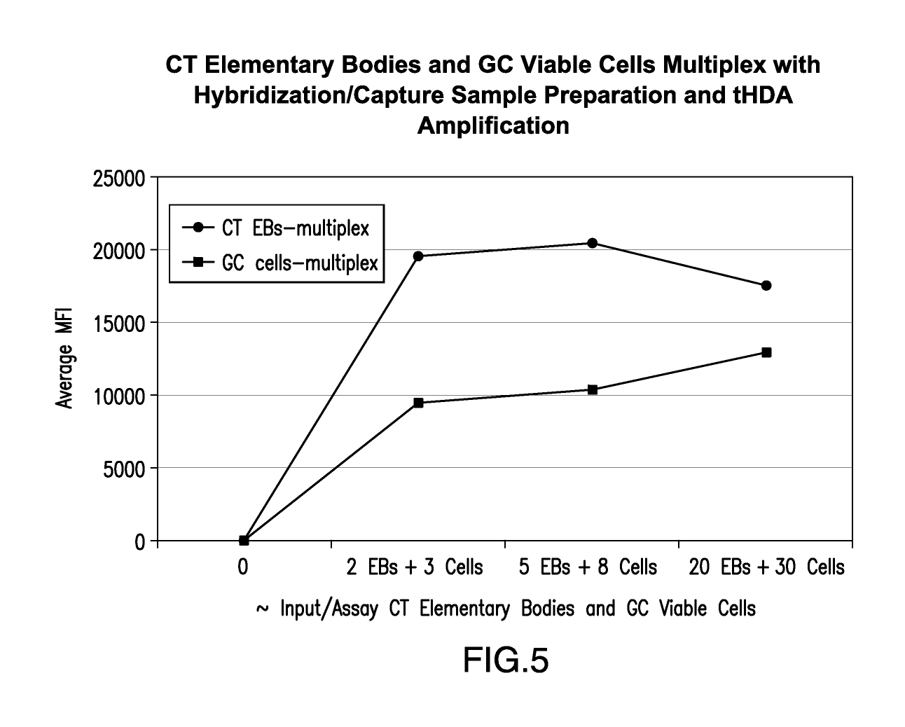

The results show that both CT Elementary Bodies (EB) and Neisseria gonorrhoeae cells can be detected in multiplex using RNA oligonucleotide probes in FE-HC before tDHA. A limitation for detection of CT/NG using hybrid capture followed by tHDA with Luminex detection can be 2 Chlamydia cells and 3 Gonorrhea cells per assay with S/N>100. The results also show that tHDA can be performed on crude samples and has the potential can be used as a diagnostic tool. (See FIG. 5).

### Example 2c

### Detection of NG Genomic DNA by tHDA with Hybrid Capture Sample Preparation and Real-Time or Endpoint Fluorescent Detection

The reaction conditions are generally set forth in Example 2a. 22 synthetic 30 nt RNA oligonucleotide probes specific to the NG opaD gene were used for capturing the NG target nucleic acid.

The helicase preparation comprised 4 mM MgSO₄; 40 mM NaCl; 0.4 mM dNTP; 3 mM dATP; 5 U rBST; and 0.5 U Helicase. In this experiment, opaD_Forward (SEQ ID NO. 221) and reverse primers (SEQ ID NO. 222) were used in concentrations of 40 nM and 180 nM, respectively. In addition, opaDv F7 primer (5′-GTTCATCCGCCATATTGTGTTG-3′, SEQ ID NO. 223) and opaDv R7 primer (5′-CACTGATGTTGAAGGACGGATTAT-3′, SEQ ID NO. 224) were else used in concentrations of 40 nM and 140 nM, respectively. For detection, the opaD-specific TaqMan Probe at a concentration of 40 nM was used.

For detection, a real-time curve with 0.2% EvaGreen and endpoint detection with opaD_b1TEX was used.

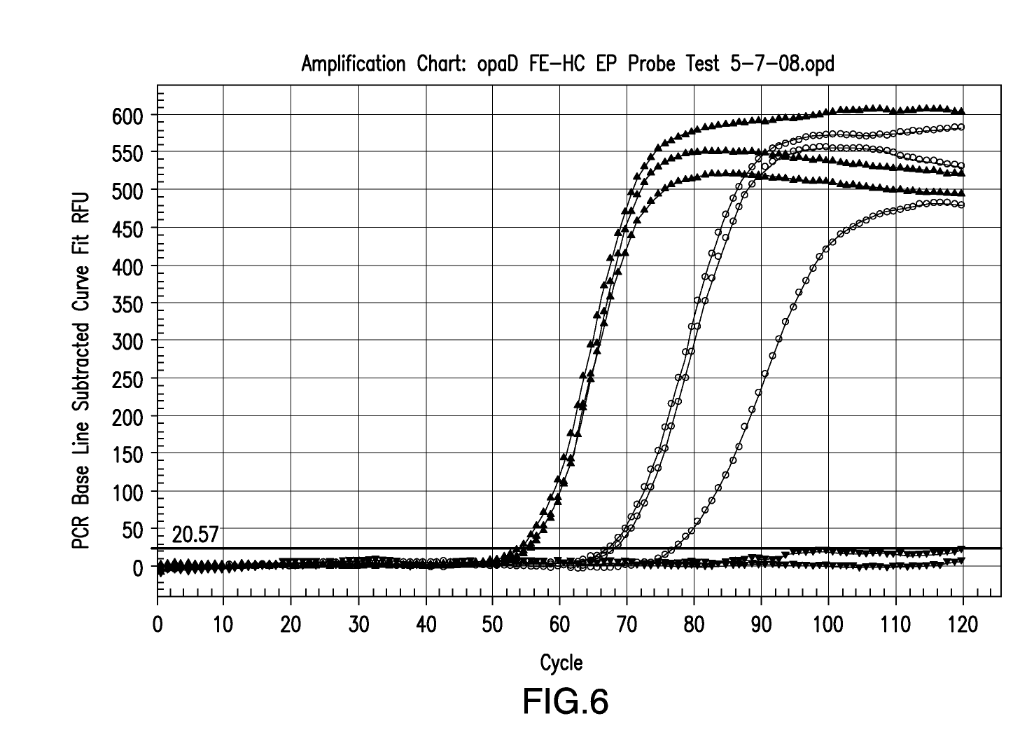

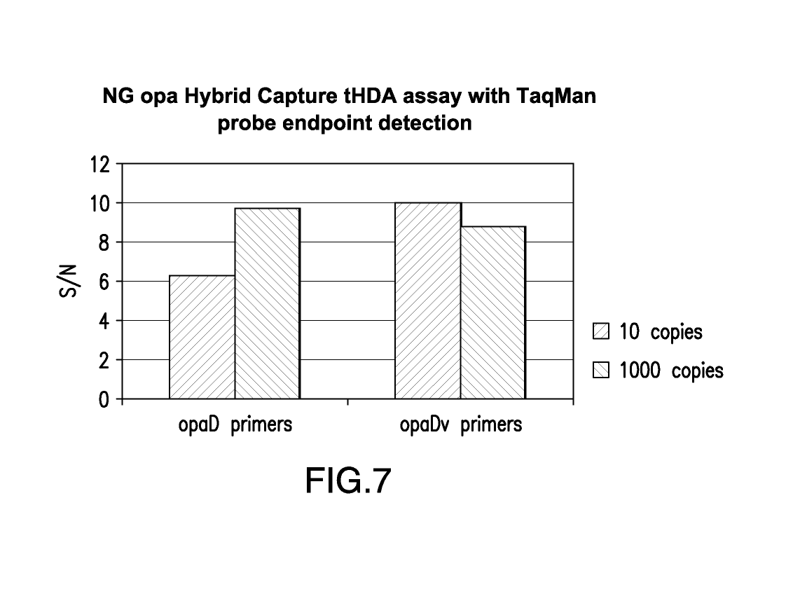

The results showed that hybrid capture sample preparation is compatible with real-time and endpoint tHDA assay with TaqMan probes as well as EvaGreen dyes. The results showed that the amplification mixture can be added to the captured duplexes and that no elution of captured duplexes on HC-beads is required. 100% of the capture duplexes can be used in tHDA reaction without significant inhibition of the reaction. (See FIGS. 6 and 7).

### Example 3

### Modified Capture Probe: Evaluation of Beacon-Like TaqMan Probe in Real-Time and Endpoint opaD tHDA Assays

A modified TaqMan probe was designed for NG opa genes' target. The modified TaqMan probe was 25 nt and was designed to be complementary to the opaD gene sequence of NG target with exception of one additional nucleotide (G-tail) at the 3′ end of the probe. The addition of this G nucleotide helped to create a stem-loop structure to ensure a low background signal for this probe. Endpoint results using classical and modified TaqMan probes for NG opa tHDA assay are shown here.

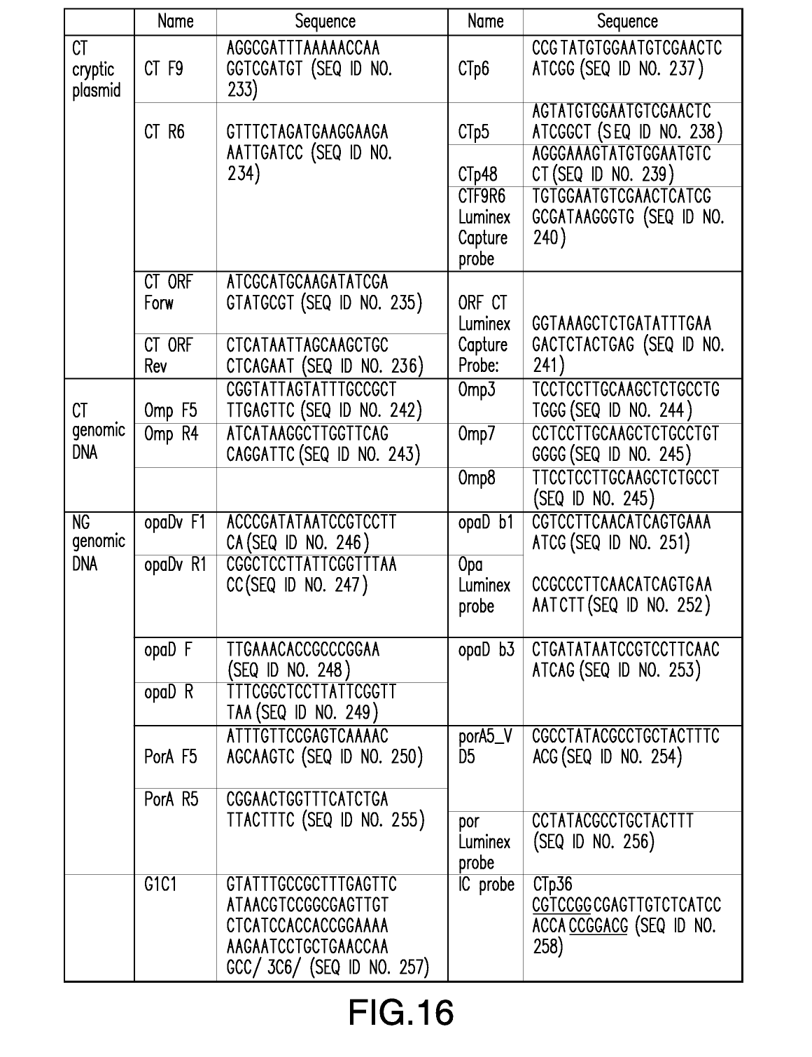

tHDA was carried out with NaOH denaturation of NG genomic DNA. NG genomic DNA of 0, 10, 100, 10³ copies/assay were used. The helicase preparation comprised 4 mM MgSO₄; 40 mM NaCl; 0.4 mM dNTP; 3 mM dATP; 5 U rBST; 0.5 U Helicase; and 1% DMSO. opaD_Forward (SEQ ID NO. 221) and reverse primers (SEQ ID NO. 222) were used in concentrations of 40 nM and 120 nM, respectively. For detection, a linear opaD probe (FAM) as well as a modified TaqMan Probe were used. 80 nm of each probe was used. The modified TaqMan Probe “opab1 modified TaqMan probe” is shown in FIG. 16. Real time tHDA was carried out for 120 cycles at 65° C.

The results are shown here in Tables 8 and 9.

TABLE 9
Modified Opa TaqMan probe

| | Ave | | |
|---|---|---|---|
| Target input | RFU | % CV | S/N |
| NTC | 62 | 11 | |
| 10 Copies | 541 | 85 | 8.7 |
| 100 Copies | 803 | 22 | 12.9 |
| 1000 Copies | 1119 | 6 | 18.1 |

TABLE 8
Opa TaqMan probe

| | Ave | | |
|---|---|---|---|
| Target input | RFU | % CV | S/N |
| NTC | 691 | 8 | |
| 10 Copies | 975 | 23 | 1.4 |
| 100 Copies | 2013 | 4 | 2.9 |
| 1000 Copies | 2066 | 7 | 3.0 |

The results show that the use of a modified opabl TaqMan probe for endpoint tHDA assay resulted in a significant increase of S/N values due to a lower background. 10 copies of NG genomic DNA were detected in tHDA assay with the modified TaqMan probe.

### Example 4

### Additives to tHDA

Example 4 demonstrates the beneficial effect of Sorbitol/DMSO combination on several tHDA assays: NG1/NG2 opa/por (example 4a), CT1/CT2/NG 3-plex (example 4b) and CT1/CT2/NG/IC 4-plex (example 4c). Endpoint fluorescence data, generated with TaqMan probes, are presented for all three examples.

### Example 4a

### Additives in NG1/NG2 Opa/Por Duplex Reaction

Reactions were carried out as described above with the exception of the changes described herein. The target nucleic acids used in the described reactions was NG genomic DNA, at concentrations of 0, 10, 102 and 105 copies/assay. Three replicates of each target input were used. The tHDA reaction conditions comprised: 0.15M Sorbitol, 1.25% DMSO, 3.5 mM MgSO4, 40 mM NaCl; 0.4 mM dNTP; 3 mM dATP; 5 U rBST; 0.5 U Helicase; (25 ul reaction). The Control reaction was carried out in the absence of any additives. For detection, TaqMan Probe: OpaD b15_Tex and porA5_VD5_Tye665 (80 nM each) were used. opaDv F1_{—}6/R1 and porA F5/R5 (40/120 nM) primers were used at the indicated concentrations.

The results of these experiments showed that DMSO with sorbitol increased signal to noise ratios for both targets in this duplex tHDA assay with endpoint fluorescence detection. Sensitivity of the assay was 10 copies target input for both targets with the addition of additives.

### Example 4b

### Additives in CT1/CT2/NG 3-Plex Reaction

Reactions were carried out as described above with the exception of the changes described herein. The target nucleic acids used in the described reactions were NG and CT genomic DNA, at concentrations of 0, 10, 10² and 10³ copies/assay. Three replicates of each target input were used. The tHDA reaction conditions comprised: 0.15M Sorbitol, 1.2% DMSO, 4 mM MgSO₄, 40 mM NaCl, 0.6 mM dNTP, 4.5 mM dATP, 20 U GST LF, 100 ng TteUvrD Helicase and 25 ng SSB (25 ul reaction). The Control reaction was carried out in the absence of any additives. For detection of NG, OpaD b1_Tex was used (60 nM). For detection of CT, the p6_Tye665 and omp3_MAX probes were used (60 nM each).

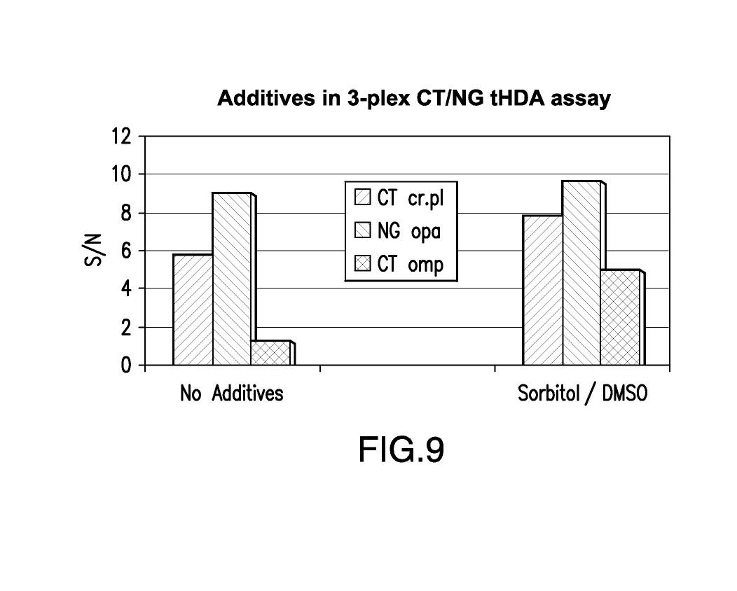

The results of these experiments showed that the addition of sorbitol/DMSO increased a signal to noise ratio for all targets in this 3-plex tHDA assay with TaqMan probes endpoint fluorescent detection. See FIG. 9.

### Example 4c

### Additives in CT1/CT2/NG/IC 4-Plex Reaction

Reactions were carried out as described above with the exception of the changes described herein. The target nucleic acids used in the described reactions were NG and CT genomic DNA, at concentrations of 0, 10, 10² and 10³ copies/assay. As a control IC: GIC1-ss DNA was used (1000 copies of GIC1). Three replicates of each target input were used.

The tHDA reaction conditions comprised: 0.15M Sorbitol, 1.2% DMSO, 4 mM MgSO₄, 40 mM NaCl, 0.6 mM dNTP, 4.5 mM dATP, 20 U GST LF, 100 ng TteUvrD Helicase and 25 ng SSB (25 ul reaction). The Control reaction was carried out in the absence of any additives.

For detection of NG, OpaD b1_Tex was used (60 nM). For detection of CT, the p6_Tye665 and omp3_MAX probes were used (60 nM each). p36 GIC1 was used to detect the control (60 nM). opaDv F/R, ompF5/R4 and CT cr.pl F9/R6 (40/120 nM) primers were used at the indicated concentrations. The omp F5R4 primer pair were used for the control.

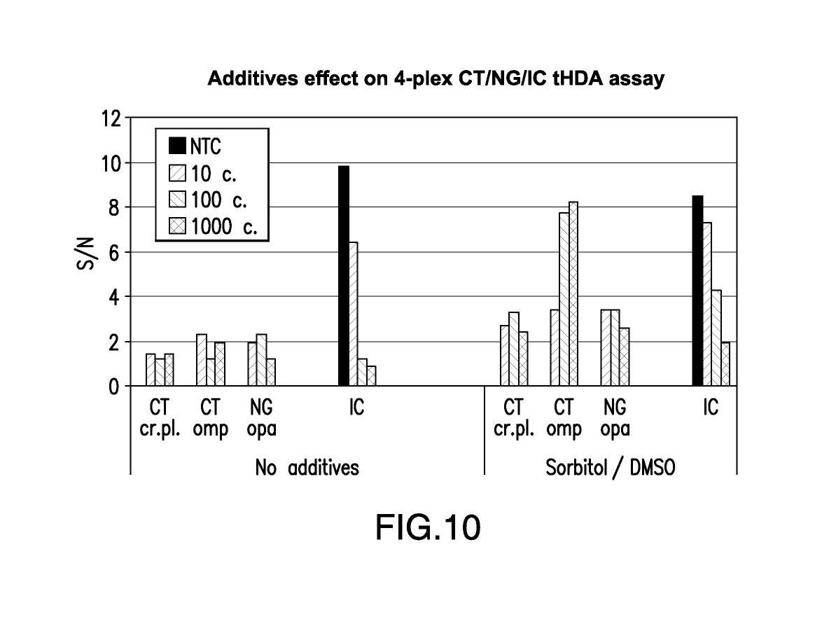

The results of these experiments showed the use of Sorbitol, in combination with DMSO, also improved the performance of this CT/NG tHDA multiplex assay. See FIG. 10.

### Example 5

### Development of a Homogeneous Multiplexed Fluorescent tHDA Assay for the Detection of N. gonorrhoeae and C. trachomatis

This Example set forth to combine Hybrid Capture sample preparation, thermophilic helicase dependent amplification (tHDA), and endpoint fluorescent detection into a highly sensitive and specific multiplexed assay for the detection of Neisseria gonorrhoeae (NG), Chlamydia trachomatis (CT), and an internal control (IC).

The target nucleic acid used for NG amplification was the multi-copy Opa gene. The target nucleic acid used for CT aplificiation was both the cryptic plasmid and the outer membrane protein (OMP) gene. Dual CT target nucleic acids and the use of a multi-copy NG target nucleic acid allow for the detection of both pathogens even if mutations or deletions are present.

Target nucleic acids in the form of DNA was extracted using Hybrid Capture® (QIAGEN Gaithersburg, Gaithersburg, Md.) sample preparation, which is compatible with various sample collection media including urine, STM, PreserveCyt® (Cytyc Corp., Bedford, Mass.), and SurePath™ (BD, Franklin Lakes, N.J.). This method utilized target specific RNA oligonucleotide probes to the target nucleic acids to create RNA:DNA double-stranded probe-target hybrids, which were then captured using capture antibodies conjugated to magnetic beads.

Following sample preparation, the captured double-stranded probe-target hybrids were directly added to a tHDA reaction. The tHDA reaction employed a helicase to unwind double stranded DNA at a single temperature. No thermal cycler was required for this reaction. Endpoint detection was then performed using dual labeled fluorescent probes.

The optimal size of specific amplification products was found to be about 70-85 bp. Asymmetric amplification conditions were helpful for endpoint fluorescent detection. The limit of detection for this experiment was determined to be ˜2 CT elementary bodies and less than 10 NG cells per mL of sample. Targets were detected in multiplex in large excess of the other target (10⁵ copies target difference). CT serovars A-K and L1-L3 were detected with comparable sensitivity. The cross-reactivity with Neisseria meningitidis and several commensal Neisseria strains was not observed.

These results support that this assay can be suitable for high-throughput automation due to its closed tube format, isothermal amplification, and rapid turn-around time.

### Example 6

### A Multiplexed Isothermal Amplification Assay for the Detection of Chlamydia trachomatis and Neisseria gonorrhoeae

This example supports the development of a sensitive, highly specific, multiplexed assay for the detection of Chlamydia trachomatis (CT) and Neisseria gonorrhoeae (NG). This example combined Qiagen's proprietary Hybrid Capture® (HC) technology (Qiagen Gaithersburg, Gaithersburg, Md.) for sample processing with isothermal helicase dependent amplification (tHDA) and endpoint fluorescence detection to develop a multiplex assay for the detection of CT and NG in clinical samples.

Up to 1 ml of sample in any of several collection media was added to a Qiagen ETU (extraction tube unit) for sample processing. The sample was lysed and DNA was denatured in alkali. A synthetic RNA oligonucleotide probe, in a neutralizing diluent, was added to the sample followed by capture beads. The sample was incubated at 50° C. to allow the synthetic RNA oligonucleotide probes to hybridize to target nucleic acid to form double-stranded probe-target hybrids. Capture antibodies conjugated to magnetic beads were then added to the double-stranded probe-target hybrids. The capture antibodies bound to the double-stranded probe-target hybrids to form captured double-stranded probe-target hybrids. The captured double-stranded probe-target hybrids were then washed to elute off any unbound nucleic acids. After several washes, the beads with attached captured double-stranded probe-target hybrids are resuspended and transferred to a reaction plate for amplification. A helicase preparation comprising a primer/detection-probe mix was added. The plate was sealed with an optical film for amplification at 65° C. for ninety minutes. After amplification, the nucleic acids were detected in a closed-tube format by endpoint fluorescence detection with dual-labeled probes.

Specifically, this assay detected two CT target nucleic acids, including the cryptic plasmid and the outer membrane protein (omp) gene. Dual targets ensure against deletion or mutation of the target sequence causing false negative results. A NG target nucleic acid was also used. Specifically the outer membrane opacity protein (opa), a multi-copy gene, served as the target nucleic acid.

From this example, as little as two CT elementary bodies, and less than ten NG cells per mL of sample were detected. Targets were detectable in multiplex, and each target was detectable in the presence of an excess (10⁵) of the other. All CT serovars A-K, and L1-L3 were amplified and detected at equivalent sensitivity. The method is suitable for the processing of samples in many different media.

As such, the combination of sequence-specific sample preparation and isothermal target amplification allows for a multiplex CT/NG assay which delivers high analytical sensitivity and specificity. The combination of short turn-around time (under three hours), isothermal reaction conditions, and closed-tube format make the assay well suited to adaptation for future high-throughput automation.

What is claimed is:

1. A method of amplifying a double stranded target nucleic acid in a helicase-dependent reaction, comprising:
(a) denaturing the target nucleic acid;
(b) contacting one or more oligonucleotide probes with the denatured target nucleic acid, wherein one or more of the oligonucleotide probes hybridize to the denatured target nucleic acid to form double-stranded probe-target hybrids;
(c) contacting the double-stranded probe-target hybrids with one or more capture antibodies wherein the one or more capture antibodies bind to the double-stranded probe-target hybrids to form captured double-stranded probe-target hybrids,
(d) removing all uncaptured nucleic acids;
(e) adding one or more oligonucleotide primers, wherein the oligonucleotide primers hybridize to the target nucleic acid;
(f) synthesizing an extension product of the oligonucleotide primers which is complementary to the target nucleic acid, by means of a DNA polymerase to form a target nucleic acid duplex;
(g) contacting the target nucleic acid duplex of step (f) with a helicase preparation and amplifying the target nucleic acid duplex in a helicase-dependent reaction.

2. The method of claim 1, wherein steps (e), (f) and (g) are carried out simultaneously.

3. The method of claim 1, wherein steps (f) and (g) are carried out simultaneously.

4. The method of claim 1, wherein the double stranded target nucleic acid is in a target nucleic acid sample.

5. The method of claim 4, wherein the sample is a blood, urine, stool, saliva, tear, bile cervical, urogenital, nasal swabs, sputum, or other biological sample.

6. The method of claim 1, wherein the double stranded target nucleic acid is isolated from a sample prior to step (a).

7. The method of claim 1, wherein amplification is isothermal.

8. The method of claim 1, wherein the polynucleotide probes are RNA.

9. The method of claim 1, wherein the helicase preparation comprises a helicase and optionally a single strand binding protein.

10. The method of claim 1, wherein the helicase preparation comprises a helicase and a single strand binding protein (SSB) unless the helicase preparation comprises a thermostable helicase wherein the single strand binding protein is optional.

11. The method of claim 1, wherein the amplification does not occur in the absence of a helicase as determined by gel electrophoresis.

12. The method of claim 1, wherein steps (e) through (g) are conducted in a homogenous assay.

13. The method of claim 1, wherein step (a) further comprises heating the target nucleic acid to denature the target nucleic acid.

14. The method of claim 11, wherein step (a) further comprises incubating the target nucleic acid in the presence of NaOH prior to step (b) as step (a).

15. The method of claim 11, wherein step (a) further comprises incubating the target nucleic acid at 65° C. for 10 minutes in the presence of 50 mM NaOH prior to step (b).

16. The method of claim 1, wherein helicase preparation comprises an additive.

17. The method of claim 16, wherein the additive is selected from the group consisting of sugars, chaperones, proteins, saccharides, amino acids, polyalcohols, and their derivatives, other osmolytes, amino acid derivatives, and chaperone proteins.

18. The method of claim 16, wherein the additive is selected from the group consisting of DMSO, betaine, sorbitol, dextran sulfate and mixtures thereof.

19. The method of claim 18, wherein DMSO is used at a final concentration of between 1 and 2%

20. The method of claim 18, wherein betaine is used at a final concentration of 0.1M-0.5M.

21. The method of claim 18, wherein sorbitol is used at a final concentration of 0.1M-0.3 M.

22. The method of claim 18, wherein dextran sulfate is used at a final concentration of 10 pM-1 nM.

23. The method of claim 1, wherein the hybrid capture antibodies comprise a magnetic bead.

24. The method of claim 1, wherein one or more of the oligonucleotide primers are present in different concentrations.

25. The method of claim 1, further comprising detecting the target nucleic acid.

26. The method of claim 1, wherein the method comprises adding a detection label.

27. The method of claim 26, wherein the detection label is added during or after step (e), (f) or (g).

28. The method of claim 25, wherein the target nucleic acid is detected both during and after the amplification reaction.

29. The method of claim 25, wherein the target nucleic acid is detected during the amplification reaction.

30. The method of claim 25, wherein the target nucleic acid is detected after the amplification reaction.

31. The method of claim 25, wherein steps (e) through (g) and the detection are carried out in a homogenous assay.

32. The method of claim 25, wherein the target nucleic acid is detected by end point fluorescent detection.

33. The method of claim 26, wherein the detection label is a modified TaqMan probe.

34. The method of claim 33, wherein the modified TaqMan probe has a short tail at 3′-end of the modified TaqMan probe complementary to the 5′-end modified TaqMan probe.

35. The method of claim 34, wherein the short tail of the modified TaqMan probe is not complementary to the target.

36. The method of claim 34, wherein the short tail of the modified TaqMan probe is also complementary to the target.

37. The method of claim 33, wherein the modified TaqMan probe has a short tail at 5′-end of the modified TaqMan probe complementary to the 3′-end modified TaqMan probe.

38. The method of claim 37, wherein the short tail of the modified TaqMan probe is not complementary to the target.

39. The method of claim 37, wherein the short tail of the modified TaqMan probe is also complementary to the target.

40. A method of amplifying a single stranded target nucleic acid in a helicase-dependent reaction, comprising:
(a) contacting one or more oligonucleotide probes with the single stranded target nucleic acid, wherein one or more of the oligonucleotide probes hybridize to the target nucleic acid to form double-stranded probe-target hybrids;
(b) contacting the double-stranded probe-target hybrids with one or more capture antibodies, wherein the one or more of capture antibodies bind to the double-stranded probe-target hybrids to form captured double-stranded probe-target hybrids,
(c) removing all uncaptured nucleic acids;
(d) adding one or more oligonucleotide primers, wherein the oligonucleotide primers hybridize to the target nucleic acid;
(e) synthesizing an extension product of the oligonucleotide primers which is complementary to the target nucleic acid, by means of a DNA polymerase to form a target nucleic acid duplex;
(f) contacting the target nucleic acid duplex of step (e) with a helicase preparation and amplifying the target nucleic acid duplex in a helicase-dependent reaction.

41. The method of claim 40, wherein the single stranded target nucleic acid is DNA.

42. The method of claim 40, wherein the single stranded target nucleic acid is cDNA and wherein the cDNA is produced from reverse transcribing a target mRNA.

43. The method of claim 40, wherein steps (e), (f) and (g) are carried out simultaneously.

44. The method of claim 40, wherein steps (f) and (g) are carried out simultaneously.

45. The method of claim 40, wherein the single stranded target nucleic acid is in a target nucleic acid sample.

46. The method of claim 45, wherein the sample is a blood, urine, stool, saliva, tear, bile cervical, urogenital, nasal swabs, sputum, or other biological sample.

47. The method of claim 40, wherein amplification is isothermal.

48. The method of claim 41, wherein the polynucleotide probes are RNA.

49. The method of claim 40, wherein one or more of the oligonucleotide primers are present in different concentrations.

50. The method of claim 40, wherein the helicase preparation comprises a helicase and optionally a single strand binding protein.

51. The method of claim 40, wherein the helicase preparation comprises a helicase and a single strand binding protein (SSB) unless the helicase preparation comprises a thermostable helicase wherein the single strand binding protein is optional.

52. The method of claim 40, wherein the amplification does not occur in the absence of a helicase as determined by gel electrophoresis.

53. The method of claim 40, wherein steps (e) through (g) are conducted in a homogenous assay.

54. The method of claim 40, wherein helicase preparation comprises an additive.

55. The method of claim 54, wherein the additive is selected from the group consisting of sugars, chaperones, proteins, saccharides, amino acids, polyalcohols, and their derivatives, other osmolytes, amino acid derivatives, and chaperone proteins.

56. The method of claim 54, wherein the additive is selected from the group consisting of DMSO, betaine, sorbitol, dextran sulfate and mixtures thereof.

57. The method of claim 56, wherein DMSO is used at a final concentration of between 1 and 2%

58. The method of claim 56, wherein betaine is used at a final concentration of 0.1M-0.5M.

59. The method of claim 56, wherein sorbitol is used at a final concentration of 0.1M-0.3 M.

60. The method of claim 56, wherein dextran sulfate is used at a final concentration of 10 pM-1 nM.

61. The method of claim 40, wherein the hybrid capture antibodies comprise a magnetic bead.

62. The method of claim 40, further comprising detecting the target nucleic acid.

63. The method of claim 62, wherein steps (e) through (g) and the detection are carried out in a homogenous assay.

64. The method of claim 40, wherein the method comprises adding a detection label.

65. The method of claim 64, wherein the detection label is added during or after step (e), (f) or (g).

66. The method of claim 62, wherein the target nucleic acid is detected both during and after the amplification reaction.

67. The method of claim 62, wherein the target nucleic acid is detected during the amplification reaction.

68. The method of claim 62, wherein the target nucleic acid is detected after the amplification reaction.

69. The method of claim 68, wherein the target nucleic acid is detected by end point fluorescent detection.

70. The method of claim 64, wherein the detection label is a modified TaqMan probe.

71. The method of claim 70, wherein the modified TaqMan probe has a short tail at 3′-end of the modified TaqMan probe complementary to the 5′-end modified TaqMan probe.

72. The method of claim 70, wherein the modified TaqMan probe has a short tail at 5′-end of the modified TaqMan probe complementary to the 3′-end modified TaqMan probe.

73. The method of claim 71, wherein the short tail of the modified TaqMan probe is not complementary to the target.

74. The method of claim 72, wherein the short tail of the modified TaqMan probe is not complementary to the target.

75. The method of claim 71, wherein the short tail of the modified TaqMan probe is also complementary to the target.

76. The method of claim 72, wherein the short tail of the modified TaqMan probe is also complementary to the target.

77. The method of claim 40, wherein the single stranded target nucleic acid is RNA.

78. The method of claim 77, wherein the one or more oligonucleotide probes are DNA probes.

79. A method of amplifying two double stranded target nucleic acids in a single helicase-dependent reaction, wherein the two double stranded target nucleic acids comprise a first and a second double stranded target nucleic acids comprising:
(a) denaturing the target nucleic acids;
(b) contacting the first denatured target nucleic acid with one or more oligonucleotide probes wherein the oligonucleotide probes hybridize to the first denatured target nucleic acid to form first target double-stranded probe-target hybrids, and contacting the second denatured target nucleic acid with one or more oligonucleotide probes wherein the oligonucleotide probes hybridize to the second denatured target nucleic acid to form second target double-stranded probe-target hybrids;
(c) contacting the first and second double-stranded probe-target hybrids with one or more capture antibodies, wherein the one or more capture antibodies bind to the first and second double-stranded probe-target hybrids to form captured first and second double-stranded probe-target hybrids,
(d) removing all uncaptured nucleic acids;
(e) adding one or more first target oligonucleotide primers, wherein the first target oligonucleotide primers hybridize to the first target nucleic acid and adding one or more second target oligonucleotide primers, wherein the second target oligonucleotide primers hybridize to the second target nucleic acid;
(f) synthesizing extension products of the first and second target oligonucleotide primers which are complementary to the first and second target nucleic acids, respectively, by means of a DNA polymerase to form first and second target nucleic acid duplexes;
(g) contacting the first and second target nucleic acid duplexes of step (f) with a helicase preparation and amplifying the target nucleic acid duplexes in a helicase-dependent reaction, wherein the helicase preparation comprises one or more primers that hybridize to the first target nucleic acid and further comprises one or more primers that hybridize to the second target nucleic acid.
